(19) United States
(12) Patent Application Publication
(10) Pub. No.: US 2026/0083722 A1
(43) Pub. Date: Mar. 26, 2026

(54) BIFUNCTIONAL DEGRADERS COMPRISING ELECTROPHILIC PROTACS THAT ENGAGE DCAF1 AND PHARMACEUTICAL COMPOSITIONS COMPRISING THE SAME

(71) Applicant: THE SCRIPPS RESEARCH INSTITUTE, La Jolla, CA (US)

(72) Inventors: Benjamin F. CRAVATT, La Jolla,, CA (US); Yongfeng TAO, San Diego, CA (US); Xiaoyu ZHANG, Glenview, IL (US); Dave REMILLARD, San Diego, CA (US); Ekaterina VINOGRADOVA, New York, NY (US); Minoru YOKOYAMA, Wakayama (JP); Bruno MELILLO, La Jolla, CA (US); Stuart SCHREIBER, Boston, MA (US)

(21) Appl. No.: 18/866,614

(22) PCT Filed: May 19, 2023
(86) PCT No.: PCT/US23/67210

§ 371 Date: Nov. 18, 2024

Related U.S. Application Data
(60) Provisional application No. 63/343,583, filed on May 19, 2022.

(51) Int. Cl. A61K 31/4709; A61K 31/4725; A61K 31/501; A61K 31/506; A61K 31/541; A61K 31/551; A61K 31/635; A61K 47/54; A61K 47/64; C07D 401/12; C07D 401/14; C07D 405/14

(52) U.S. Cl. CPC A61K 31/4709; A61K 31/4725; A61K 31/501; A61K 31/506; A61K 31/541; A61K 31/551; A61K 31/635; A61K 47/545; A61K 47/64; C07D 401/12; C07D 401/14; C07D 405/14

(57) ABSTRACT

Disclosed are bifunctional degraders comprising electrophilic PRO-TACs that engage DCAF1 and pharmaceutical compositions comprising the same. The bifunctional degraders are of Formula A-B-C wherein, A is a ligand to a protein of interest, B is a linker that is a bond or a chemical linker that is chemically linked to A and C, and C is a ligand to the E3 ligase substrate receptor DCAF1, wherein the protein of interest is any protein having a ligand that can form a covalent bond with the linker B; and C comprises an azetidinyl acrylamide that forms a covalent bond with C1113 of DCAF1 through a Michael addition reaction.

### CROSS REFERENCE TO RELATED APPLICATION

This application claims priority to U.S. provisional patent application No. 63/343,583, which was filed on May 19, 2022, and which is hereby incorporated by reference in its entirety.

### GOVERNMENT SUPPORT

This invention was made with government support under grant numbers CA231881 and CA248715 awarded by the National Institute of Health. The government has certain rights in the invention.

### FIELD OF INVENTION

This invention relates to bifunctional degraders comprising electrophilic PROTACs that engage DCAF1.

### BACKGROUND OF THE DISCLOSURE

Most small molecules affect the functions of proteins through binding-mediated agonism or antagonism. Targeted protein degradation has recently emerged as a distinct type of pharmacology wherein small molecules induce the formation of complexes between a substrate protein and a component of the ubiquitin-proteasome system (typically, an E3 ligase) to promote the physical turnover of the substrate protein¹⁻³. Targeted protein degradation is often enacted by compounds termed PROTACs (proteolysis-targeting chimeras), which are heterobifunctional molecules that contain two independent recognition units-one that binds the substrate protein and the other that binds an E3 ligase-connected by a linker to form a ternary complex that brings the substrate protein into proximity of the E3 ligase, resulting in substrate ubiquitination and proteasome-mediated degradation^{2, 4}. PROTACs have the potential to address gaps in chemical probe and drug development by, for instance, providing a means to i) eliminate multidomain or multifunctional proteins for which small-molecule antagonism proves insufficient to block the full scope of protein activities, and ii) convert silent ligand-protein interactions into functional (degradation) outcomes². The successful design of PROTACs depends on identifying ligands for E3 ligases. Even though human cells express hundreds of E3 ligases, small-molecule ligands have been discovered so far for only a small number of these proteins^{2, 8}, and most PROTACs utilize one of two E3 ligases-cereblon (CRBN) or VHL^{2, 9-10}. Recent work indicates that CRBN and VHL display distinct and restricted substrate specificities for executing targeted protein degradation¹¹⁻¹⁴

There is therefore a need to identify ligands for additional E3 ligases for realizing the full potential of targeted protein degradation.

### SUMMARY OF THE DISCLOSURE

Some embodiments described herein provide a bifunctional degrader of Formula (I)

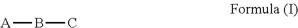

wherein, A is a ligand to a protein of interest, B is a linker that is a bond or a molecular linker that is chemically linked to A and C, and C is a ligand to the E3 ligase substrate receptor DCAF1, wherein:
the protein of interest is any protein having a ligand that can form a covalent bond with the linker B; and
C comprises an azetidinyl acrylamide that forms a covalent bond with C1113 of DCAF1 through a Michael addition reaction.

Some embodiments described herein also provide a DCAF1 protein-probe adduct, wherein the probe binds to cysteine residue C1113 of DCAF1, and wherein the probe comprises an azetidinyl acrylamide moiety.

Some embodiments described herein also provide a compound of Formula (I)

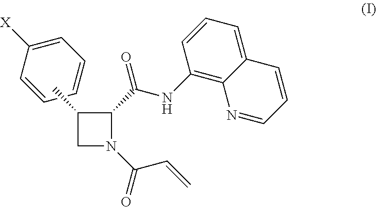

or a pharmaceutically acceptable salt thereof, wherein:
X is selected from the group consisting of halo,

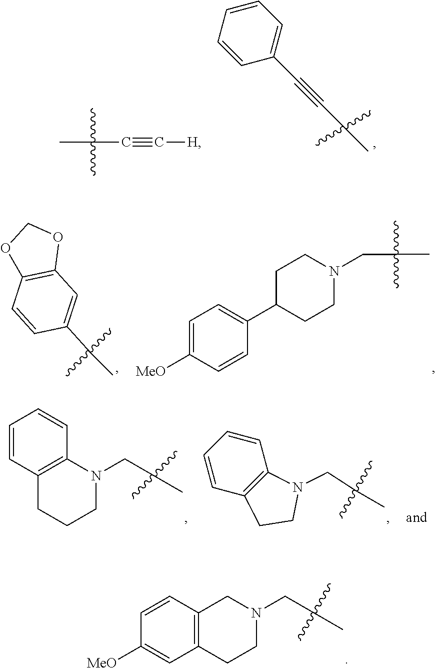

### BRIEF DESCRIPTION OF THE FIGURES

FIG. 1 shows the chemical proteomic discovery of a stereo- and site-selective covalent ligand for DCAF1. (A) Structures of a set of stereochemically defined azetidine acrylamides MY-1A (1), MY-1B (2), MY-3A (3), and MY-3B (4). (B) Heat map showing cysteines that were substantially engaged by azetidine acrylamides (>75% by at least one compound) in human T cells (20 μM compound, 3 h) as determined by MS-ABPP using an iodoacetamide desthiobiotin (IA-DTB) probe following previously described methods²¹. Red arrow marks DCAF1_C1113. (C) MS-ABPP quantification of IA-DTB labeling of DCAF1_C1113 from T cells treated with the indicated azetidine acrylamides (20 μM, 3 h) relative to control T cells treated with DMSO. (D) MS-ABPP quantification of IA-DTB labeling of the indicated cysteines in DCAF1 from T cells treated with MY-1B (20 μM, 3 h) relative to control T cells treated with DMSO. For (B-D), data represent average values (for C and D, average values±SEM) from three independent experiments each performed with two technical replicates, where cysteines were required to have been quantified in at least two experiments for interpretation.

FIG. 2 that azetidine acrylamides stereo- and site-selectively engage recombinant DCAF1_C1113. (A) Domain map of DCAF1 with the region used for recombinant protein studies marked in a bracket (a.a.'s 1046-1396) and C1113 highlighted in red. (B) Crystal structure of a DCAF1-Vpx complex (pdb: 5AJA). DCAF1 (a.a.'s 1046-1396) is shown in cyan, Vpx is shown in yellow, and C1113, which is located at the DCAF1-Vpx interface is shown in red. (C) Structures of alkyne-modified azetidine acrylamides MY-11A (5) and MY-11B (6). (D) Gel-ABPP showing the concentration-dependent reactivity of MY-11A and MY-11B (1 h) reactivity with recombinant, purified DCAF1-WT or the DCAF1-C1113A mutant (0.06 μg/μL of DCAF1 protein per sample) doped into HEK293T cell proteome (1 μg/μL). MY-11A/B labeling of proteins was visualized by CuAAC conjugation to an azide-rhodamine tag followed by SDS-PAGE and in-gel fluorescence scanning. Results are from a single experiment representative of two independent experiments. (E) Gel-ABPP showing the concentration-dependent effects of MY-1A and MY-1B on MY-11B reactivity with DCAF1-WT. Samples were treated with MY-1A/B for 2 h, followed by MY-11B (25 μM, 1 h) and analyzed as described in (D). Left, quantification of data presented as mean values±SEM from two independent experiments. Right, representative gel-ABPP results. (D, E) Lower images are Coomassie blue-stained gels corresponding to ABPP gels shown in upper images.

FIG. 3 shows that azetidine acrylamide-based heterobifunctional compounds stereoselectively engage DCAF1. (A) Structures of candidate electrophilic PROTACs YT41R (7), YT41S (8), YT47R (9), and YT47S (10). (B) Gel-ABPP showing the concentration-dependent effects of YT41R and YT47R on MY-11B reactivity with DCAF1-WT protein (0.06 μg/μL of DCAF1 protein per sample) doped into HEK293T cell proteome (1 μg/μL). Also shown are the effects of YT41S and YT47S tested at a single concentration (100 μM). Samples were treated with heterobifunctional compounds for 2 h, followed by MY-11B (25 μM, 1 h) and analyzed as described in FIG. 2D. Left, representative gel-ABPP results. Lower image is Coomassie blue-stained gel corresponding to ABPP gel shown in upper image. Right, quantification of data presented as mean values±SEM from two independent experiments.

FIG. 4 shows that DCAF1-directed electrophilic PROTACs promote FKBP12 degradation in a stereo- and site-selective manner. (A) Left, western blots showing concentration-dependent effects of YT47R (upper) and YT41R (lower) on FKBP12 abundance in HEK293T cells expressing HA-FBKP12 with or without co-expression of FLAG-DCAF1. Right, quantification of HA-FKBP12 abundance in the indicated experimental groups. (B) Left, western blots showing effects of YT47R versus YT47S (upper) and YT41R versus YT41S (lower) on FKBP12 abundance in HEK293T cells co-expressing HA-FBKP12 and FLAG-DCAF1. Right, quantification of HA-FKBP12 abundance in the indicated experimental groups. (C) Western blots showing effects of YT47R (left blots) and YT41R (right blots) on FKBP12 abundance in HEK293T cells co-expressing HA-FBKP12 and either FLAG-DCAF1-WT or FLAG-DCAF1-C1113A mutant. Quantification of HA-FKBP12 abundance in the indicated experimental groups is shown in the adjacent bar graphs. For (A-C), FKBP12 abundance was determined at 24 h post-treatment with compounds, and data are mean values±SEM for three independent experiments. Statistical significance was calculated with unpaired two-tailed Student's t-tests comparing YT41R/YT47R- and DMSO-treated cells. ***P<0.001, ****P<0.0001.

FIG. 5 shows that FKBP12 degradation induced by DCAF1-directed electrophilic PROTACs is dependent on the ubiquitin-proteasome system. (A) Left, western blots showing effects of proteasome (MG132) and Neddylation (MLN4924) inhibitors (1 h pre-treatment) on YT47R- and YT41R-dependent degradation of FKBP12 in HEK293T cells co-expressing HA-FBKP12 and FLAG-DCAF1. FKBP12 abundance was determined at 24 h post-treatment with YT47R or YT41R. Right, quantification of HA-FKBP12 abundance in the indicated experimental groups. (B) Left, western blots showing effects of the FKBP12 ligand SLF (20 μM) or DCAF1 ligand MY-11B (1 h pre-treatment with 20 μM SLF or 5 μM MY-11B) on YT47R- and YT41R-dependent degradation of FKBP12 in HEK293T cells co-expressing HA-FBKP12 and FLAG-DCAF1. FKBP12 abundance was determined at 24 h post-treatment with YT47R or YT41R. Right, quantification of HA-FKBP12 abundance in the indicated experimental groups. (C) Left, western blots showing effects of MY-11A or MY-11B (5 μM, 1 h pre-treatment) on YT47R- and YT41R-dependent degradation of FKBP12 in HEK293T cells co-expressing HA-FBKP12 and FLAG-DCAF1. FKBP12 abundance was determined at 24 h post-treatment with YT47R or YT41R. Right, quantification of HA-FKBP12 abundance in the indicated experimental groups. For (A-C), data are mean values±SEM for three-five independent experiments. Statistical significance was calculated with unpaired two-tailed Student's t-tests comparing YT41R/YT47R- and DMSO-treated cells. ****P<0.0001.

FIG. 6 shows the evidence for ternary complex formation and ubiquitination of HA-FKBP12 by DCAF1-directed electrophilic PROTACs. (A) Left, western blots showing effects of YT47R and YT47S on the immunoprecipitation (IP) of FLAG-DCAF1-WT or FLAG-DCAF1-C1113A with HA-FKBP12 in HEK293T cells co-expressing these proteins. Cells were treated with YT47R or YT47S (5 μM) for 2 h in the presence of MG132 (10 μM) prior to lysis and IP with an anti-HA antibody and western blotting for the indicated proteins (DCAF1, FKBP12, and ubiquitinated protein). Right, bar graphs quantifying DCAF1 (top) and ubiquitination (Ub) (bottom) in the indicated IP groups. (B) Left, western blots showing effects of YT47R and YT47S on the IP of endogenous DCAF1 with HA-FKBP12 in HEK293T cells expressing HA-FKBP12. Cells were treated with YT47R or YT47S (5 μM) for 2 h in the presence of MG132 (10 μM) prior to lysis and IP with an anti-HA antibody and western blotting for the indicated proteins (DCAF1, FKBP12). Right, bar graph quantifying DCAF1 in the indicated IP groups. For (A, B), data are mean values±SEM for three independent experiments. Statistical significance was calculated with unpaired two-tailed Student's t-tests comparing YT41R/YT47R- and DMSO-treated cells. **P<0.01, ****P<0.0001. (C) MS-ABPP quantification of IA-DTB labeling of the indicated cysteines in DCAF1 from FLAG-DCAF1-WT-expressing HEK293T cells treated with the indicated concentrations of YT47R (3 h) relative to cells treated with DMSO. Data represent average values±SEM from four independent experiments. Statistical significance was calculated with unpaired two-tailed Student's t-tests comparing compound to DMSO treated cells. *P<0.1.

Figure S1. A computational model recapitulates the stereoselective engagement of DCAF1_C1113 by MY-1B. (A) Predicted binding pose of MY-1B. Apical (left) and lateral (right) views are shown, with the latter being clipped for clarity. DCAF1_C1113 is shown in yellow and a dashed line connects C1113 sulfhydryl and acrylamide terminal carbon to illustrate a simplified nucleophile trajectory. (B) Diagram illustrating predicted interactions between MY-1B and DCAF1 residues located within 4 Å of the ligand. Green contour: hydrophobic residues, blue contour: polar residues; green lines: π-π stacking interactions, brown arrow: halogen bond. (C) Predicted binding energies of MY-1B and its stereoisomers (Schrödinger Prime MM-GBSA, best of 3 poses) and respective distances between C1113 sulfhydryl and acrylamide terminal carbon.

Figure S2. A computational model supports conjugation strategies of MY-1B core scaffold. (A) Conjugation was considered at the phenyl 4-position (solid arrow) and the acrylamide position (dashed arrow). The former was prioritized to avoid affecting the cysteine-reactivity of the parent compound. (B) Constrained docking poses of MY-1B-derived conjugate S.10 (apical view, 3 poses shown). (C) Left: Diagram illustrating predicted interactions between MY-1B conjugate S.10 and DCAF1 residues located within 4 Å of the ligand. Green contour: hydrophobic residues, blue contour: polar residues; green lines: π-π stacking interactions, brown arrow: halogen bond; grey shading denotes solvent exposure. Right: corresponding docking pose.

Figure S3. Concentration-dependent degradation of FKBP12 by DCAF1-directed electrophilic PROTACs. Top, western blots showing concentration-dependent effects of YT47R and YT41R on FKBP12 abundance in HEK293T cells co-expressing HA-FBKP12 and FLAG-DCAF1. Data are mean values±SEM for four independent experiments. Statistical significance was calculated with unpaired two-tailed Student's t-tests comparing YT41R/YT47R- and DMSO-treated cells. *P<0.1, **P<0.01, ***P<0.001, ****P<0.0001.

Figure S4. Time-dependent degradation of FKBP12 by DCAF1-directed electrophilic PROTACs. Top, western blots showing time-dependent effects of YT47R and YT41R (2 μM each) on FKBP12 abundance in HEK293T cells co-expressing HA-FBKP12 and FLAG-DCAF1. Data are mean values±SEM for four independent experiments. Statistical significance was calculated with unpaired two-tailed Student's t-tests comparing YT41R/YT47R- and DMSO-treated cells. ****P<0.0001.

Figure S5. A reversible small molecule CYCA-117-70 binds DCAF1 near C1113. Apical and lateral views are shown. C1113 is indicated in yellow for visual reference. X-ray diffraction data generated by Kimani, S. et al. (1.62-Å resolution, PDB: 7SSE).

### DETAILED DESCRIPTION OF THE DISCLOSURE

Unless otherwise stated, the following terms used in the specification and claims are defined for the purposes of this Application and have the following meaning. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents may be selected from the group consisting of halogen, —CN, —NO₂, —N₃, —SO₂H, —SO₃H, —OH, —OR^{aa}, —N(R^{bb})₂, —N(OR)R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR)₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O;
each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and
each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl) (C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂,C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

It should be noted that in hetero-atom containing ring systems described herein, there are no hydroxyl groups on carbon atoms adjacent to a N, O or S, as well as there are no N or S groups on carbon adjacent to another heteroatom. Thus, for example, in the ring:

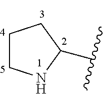 [SMILES: *C1CCCN1]

there is no —OH attached directly to carbons marked 2 and 5.

It should also be noted that tautomeric forms such as, for example, the moieties:

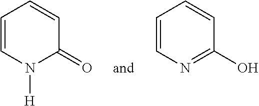 [SMILES: O=c1cccc[nH]1 ; Oc1ccccn1]

are considered equivalent unless otherwise specified.

As used herein, the term “Michael addition reaction” is well known to those of ordinary skill in the art of organic chemistry. In this reaction, a new covalent bond is formed between a portion of the Michael acceptor moiety (for example, an alpha, beta unsaturated functionality, such as an acrylamide) and a donor moiety. The Michael acceptor moiety is an electrophile and the “donor moiety” is a nucleophile (such as the sulfur atom in the SH group of cysteine)

As used herein, the term “composition” is intended to encompass a product comprising the specified ingredients in the specified amounts, as well as any product which results, directly or indirectly, from combination of the specified ingredients in the specified amounts.

“Effective amount” or “therapeutically effective amount” is meant to describe an amount of compound or a composition described herein that is effective in inhibiting the recited diseases or conditions, and thus producing the desired therapeutic, ameliorative, inhibitory and/or preventative effect.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F. with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., 2H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

The compounds described herein can also be used in combination with one or more additional therapeutic and/or prophylactic agents.

It is also possible to combine any compound of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds of the invention before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

### EMBODIMENTS

Examples of embodiments of the present application include the following:

### Embodiment 1

a bifunctional degrader of Formula (I)

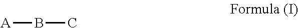

wherein, A is a ligand to a protein of interest, B is a linker that is a bond or a chemical linker that is chemically linked to A and C, and C is a ligand to the E3 ligase substrate receptor DCAF1, wherein:
the protein of interest is any protein having a ligand that can form a covalent bond with the linker B; and
C comprises an azetidinyl acrylamide that forms a covalent bond with C1113 wherein the amino acid numbering is based on DCAF1 Isoform 1 (Accession No. Q9Y4B6-1), C1112 of DCAF1, wherein the amino acid numbering is based on DCAF1 Isoform 2 (Accession No. Q9Y4B6-2), or C664 of DCAF1, wherein the amino acid numbering is based on DCAF1 Isoform 3 (Accession No. Q9Y4B6-3), through a Michael addition reaction.

### Embodiment 2

The bifunctional degrader of Embodiment 1, wherein the protein of interest is selected from the group consisting of FKBP12, BRD4, androgen receptor, estrogen receptor, IRAK4, a JAK protein, BCL-XL, BCL-2, and Stat3.

### Embodiment 3

The bifunctional degrader of Embodiment 1 or 2, wherein azetidinyl acrylamide of C forms a covalent bond with C1113 of DCAF1 through a Michal addition reaction.

### Embodiment 4

The bifunctional degrader of any one of Embodiments 1-3, wherein C has the structural Formula (C-1):

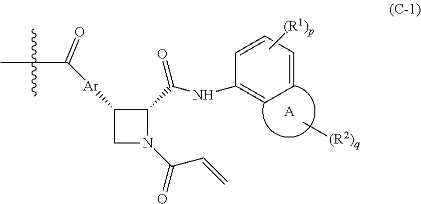

wherein:
Ar is a C₆-C₁₀ aryl, optionally substituted with 1-3 moieties selected from the group consisting of: halo, hydroxy, cyano, optionally substituted C₁-C₆ alkyl, —O—(C₁-C₆ alkyl), optionally substituted C₆-C₁₀ aryl, optionally substituted C₃-C₈ cycloalkyl, —C(═O)—(C₁-C₆ alkyl), optionally substituted-(C₁-C₃)ₙ-heterocyclylphenyl, C₆-C₁₀ aryl, —(C₁-C₃)ₙ-linked optionally substituted five- to six-membered heterocyclyl, —(C₁-C₃)ₙ-linked optionally substituted five- to six-membered heterocyclyl fused to an optionally substituted C₆-C₁₀ aryl, and —(C₁-C₃)ₙ-linked optionally substituted five- to six-membered heteroaryl;
each R¹ independently is an optional substituent selected from the group consisting of: halo, cyano, optionally substituted C₁-C₆ alkyl, —O—C₁-C₆ alkyl, optionally substituted C₆-C₁₀ aryl, optionally substituted C₃-C₈ cycloalkyl, —C(═O)(C₁-C₆ alkyl), optionally substituted five- to six-membered heterocyclyl, and optionally substituted five- to six-membered heteroaryl;
n is 0, 1, or 2;
p is 0, 1 or 2;
ring A is a five- or six-membered heteroaryl;
each R² independently is an optional substituent selected from the group consisting of: halo, cyano, optionally substituted C₁-C₆ alkyl, —O—C₁-C₆ alkyl, optionally substituted C₆-C₁₀ aryl, optionally substituted C₃-C₈ cycloalkyl, —C(═O)(C₁-C₆ alkyl), optionally substituted five- to six-membered heterocyclyl, and optionally substituted five- to six-membered heteroaryl;
q is 0, 1, or 2; and
[symbol] indicates the point of attachment to the linker B.

### Embodiment 5

The bifunctional degrader of Embodiment 4 wherein:

Ar is substituted with 1-2 substituents selected from the group consisting of halo, hydroxy, cyano, —O—C₁-C₆alkyl, -alkynylphenyl, flurophenoxy, methoxyphenyl, —((C₁-C₃)ₙ)-4-(4-methoxyphenyl) piperidine and —(C₁-C₃)ₙ-linked optionally substituted five- to six-membered heterocyclyl fused to an optionally substituted C₆-C₁₀ aryl.

### Embodiment 6

The bifunctional degrader of Embodiment 5, wherein:
the —(C₁-C₃)ₙ-linked optionally substituted five- to six-membered heterocyclyl fused to an optionally substituted C₆-C₁₀ aryl is selected from the group consisting of —((C₁-C₃)ₙ)-benzo[d][1,3]dioxolyl, —((C₁-C₃)ₙ)-1,2,3,4-tetrahydroquinoline-1-yl, —((C₁-C₃)ₙ)-1,2,3,4-tetrahydroisoquinoline-2-yl, —((C₁-C₃)ₙ)-6-methoxy-1,2,3,4-tetrahydroisoquinoline-2-yl, and —((C₁-C₃)ₙ)-indoline-1-yl; and
n is 0 or 1.

### Embodiment 7

The bifunctional degrader of any one of Embodiments 1-6, wherein Formula (C-1) has the formula (C-1a):

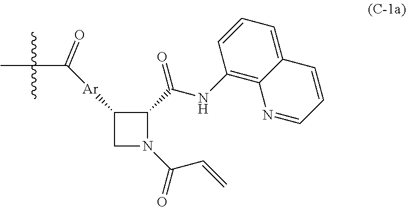

### Embodiment 8

The bifunctional degrader of any one of Embodiments 1-7, wherein

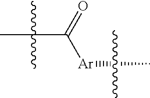

is:

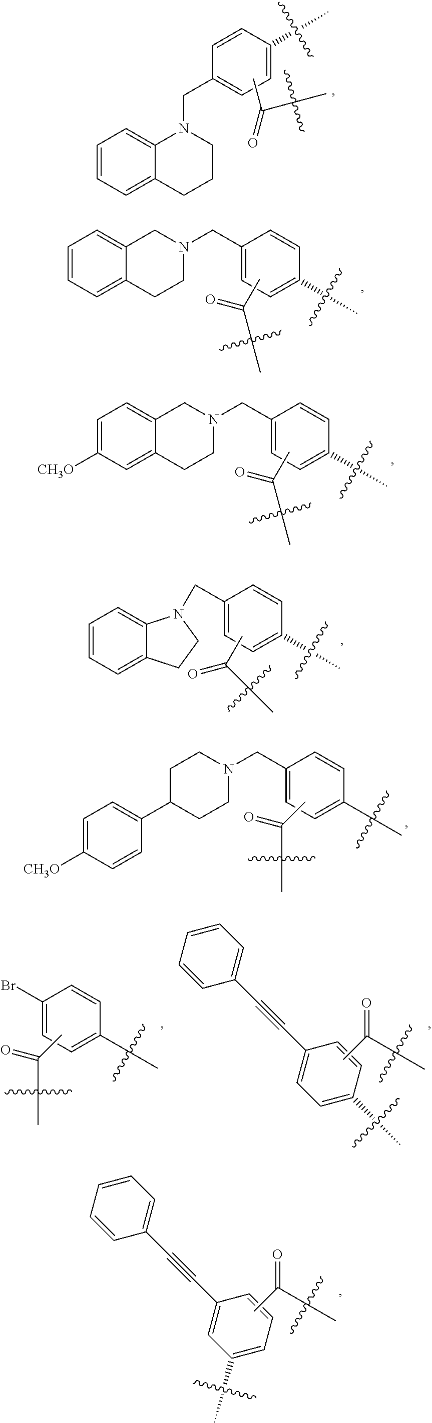

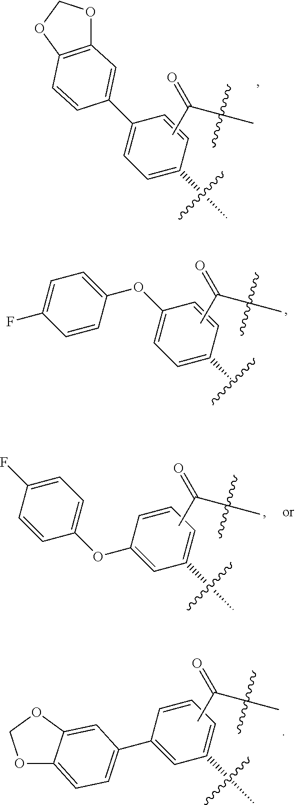

### Embodiment 9

The bifunctional degrader of any one of Embodiments 1-8, wherein linker B comprises a moiety having ethylene repeat units, the moiety having the formula (B-1)

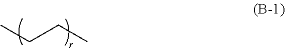

wherein r is an integer from 1 to 10; or a moiety having ethylene glycol repeat units, the moiety having the formula (B-2)

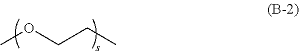

wherein s is an integer from 1 to 10.

### Embodiment 10

The bifunctional degrader of any one of Embodiments 1-9, wherein linker B

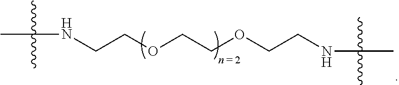

### Embodiment 10a

The bifunctional degrader of any one of Embodiments 1-9, wherein linker B is selected from the group consisting of:

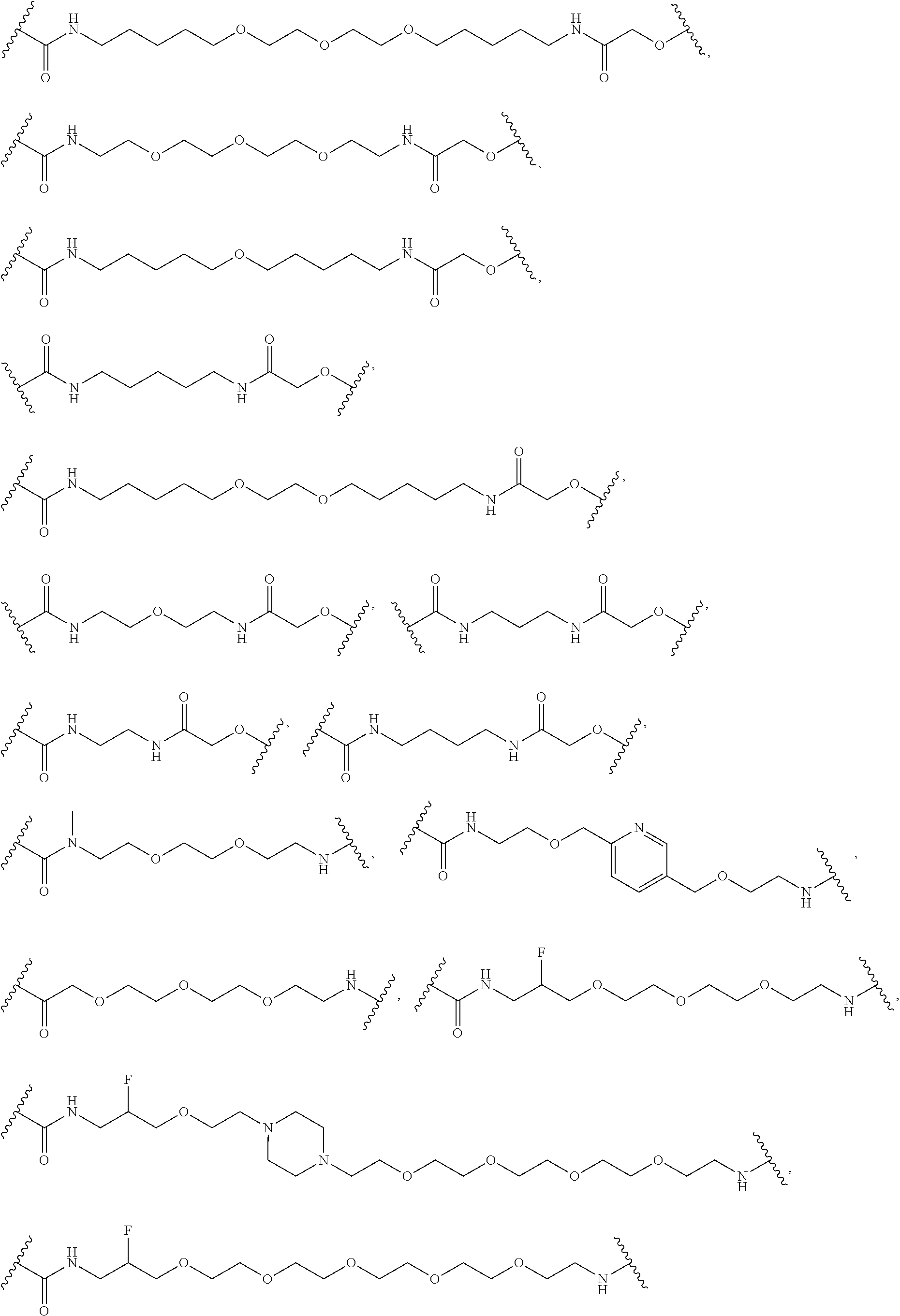

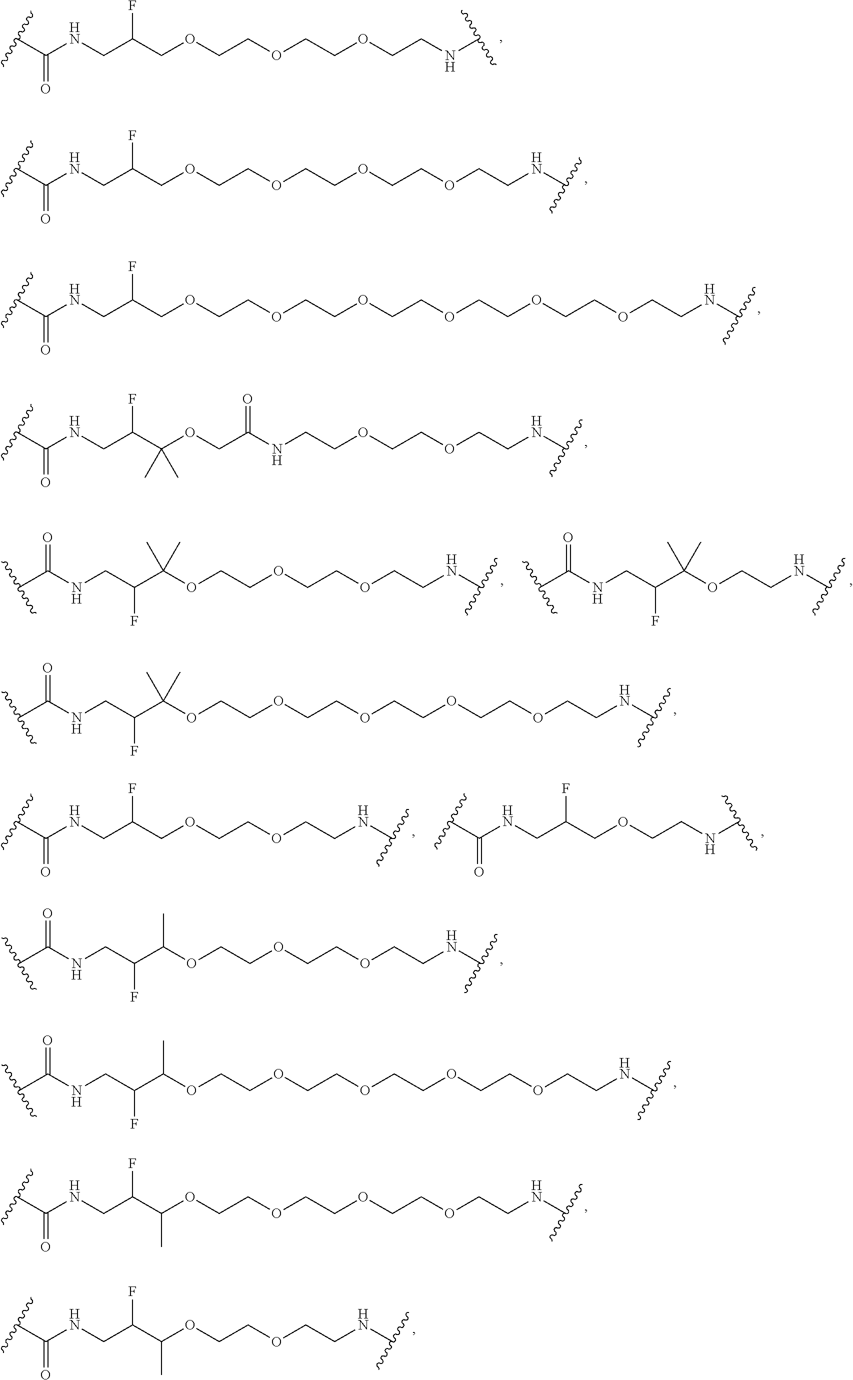

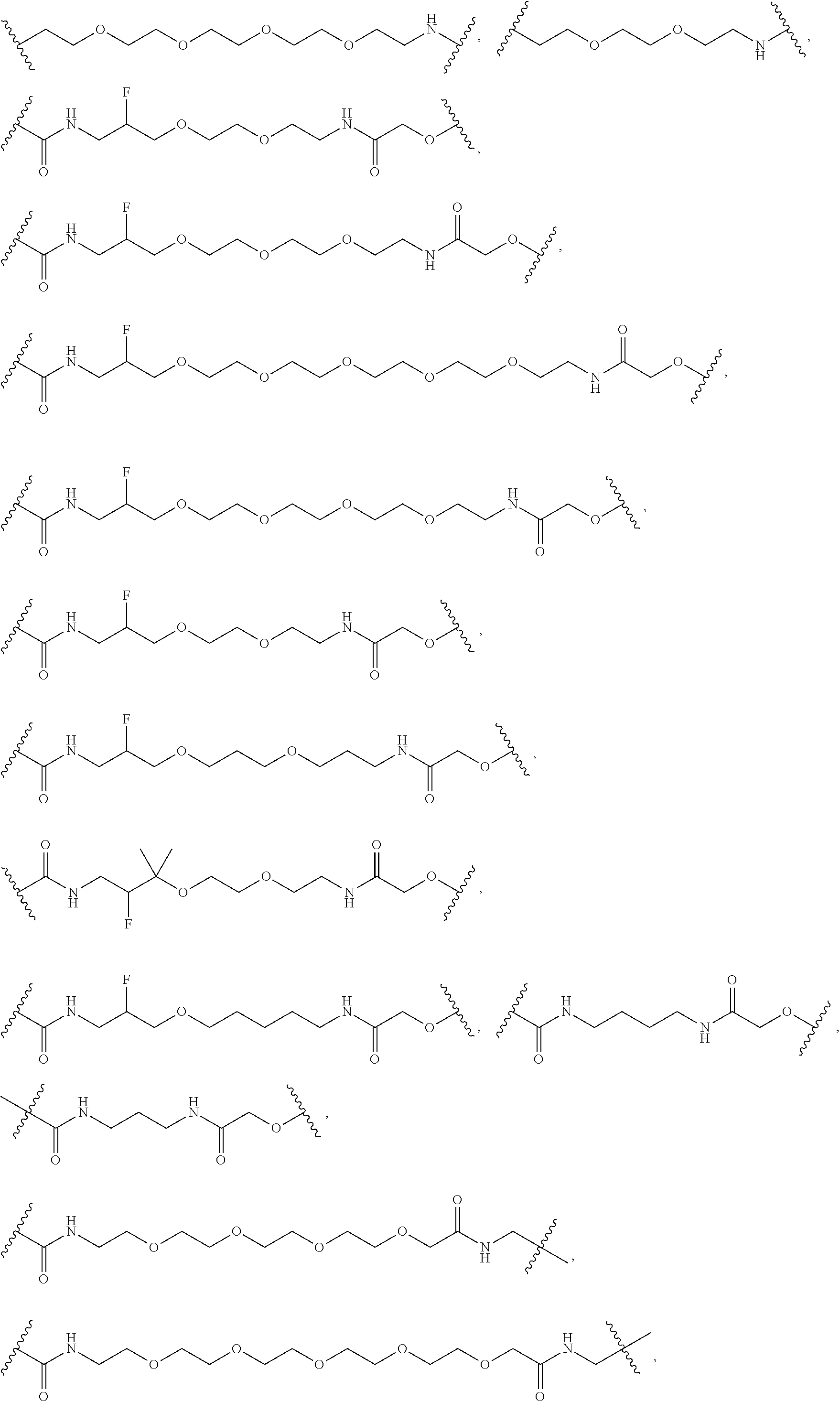

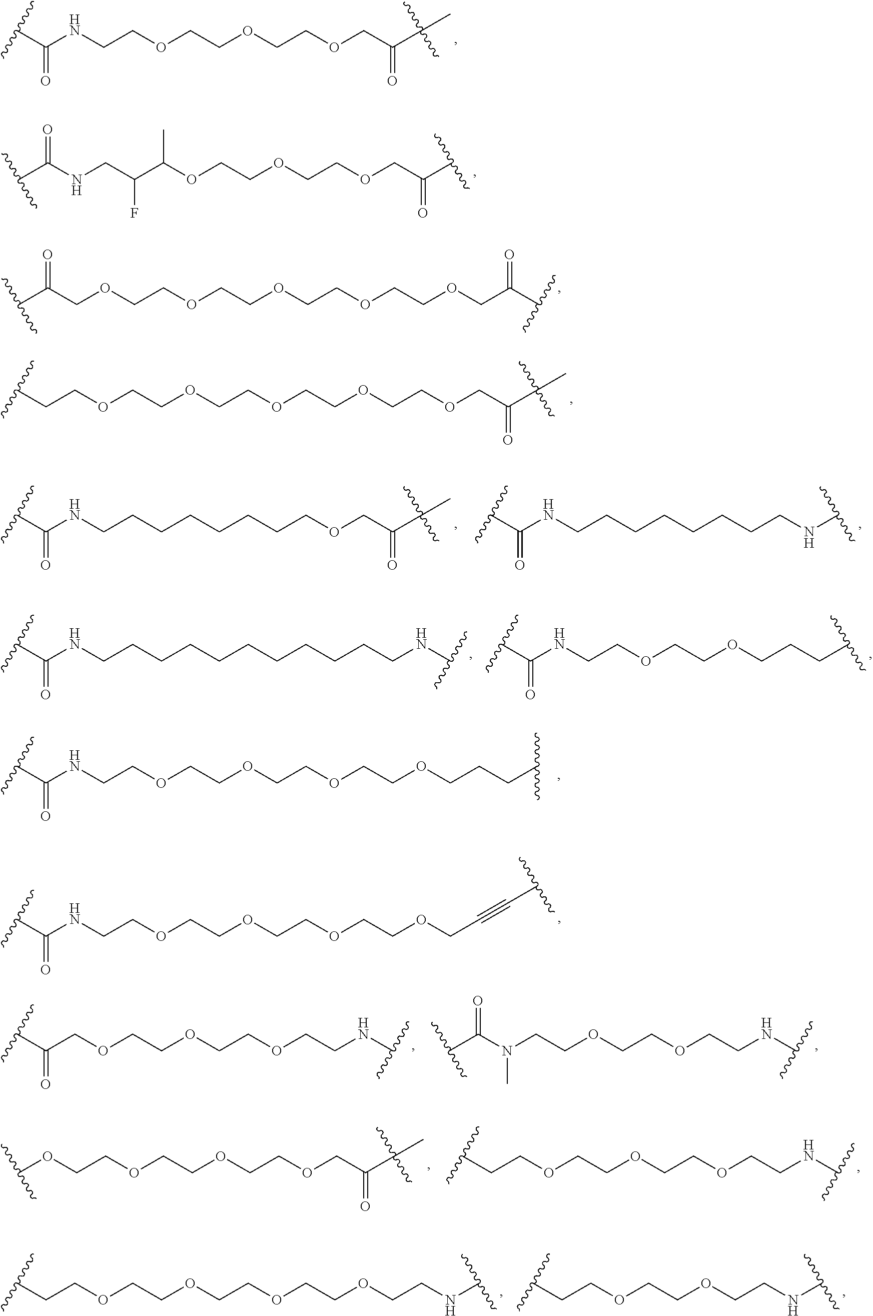

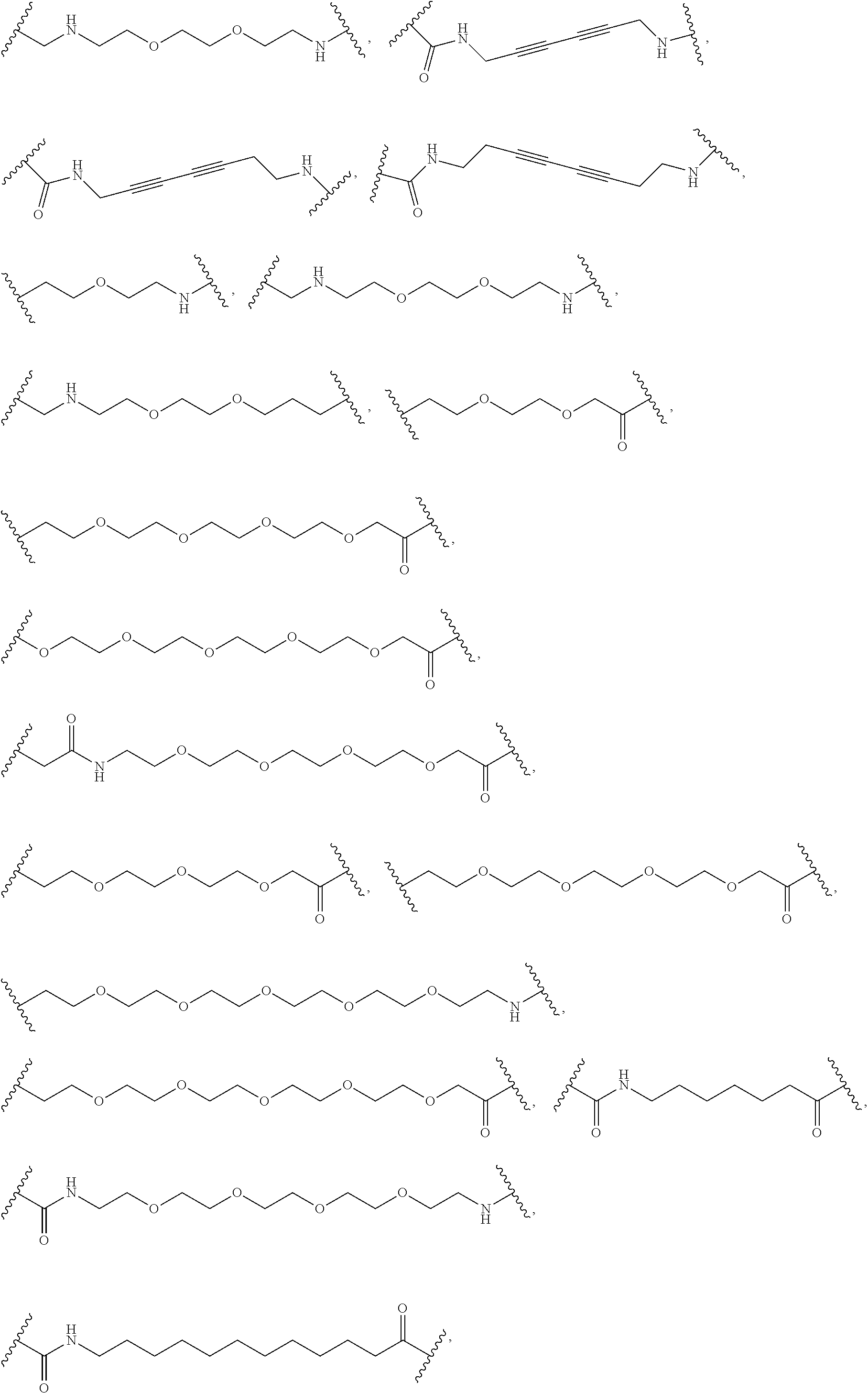

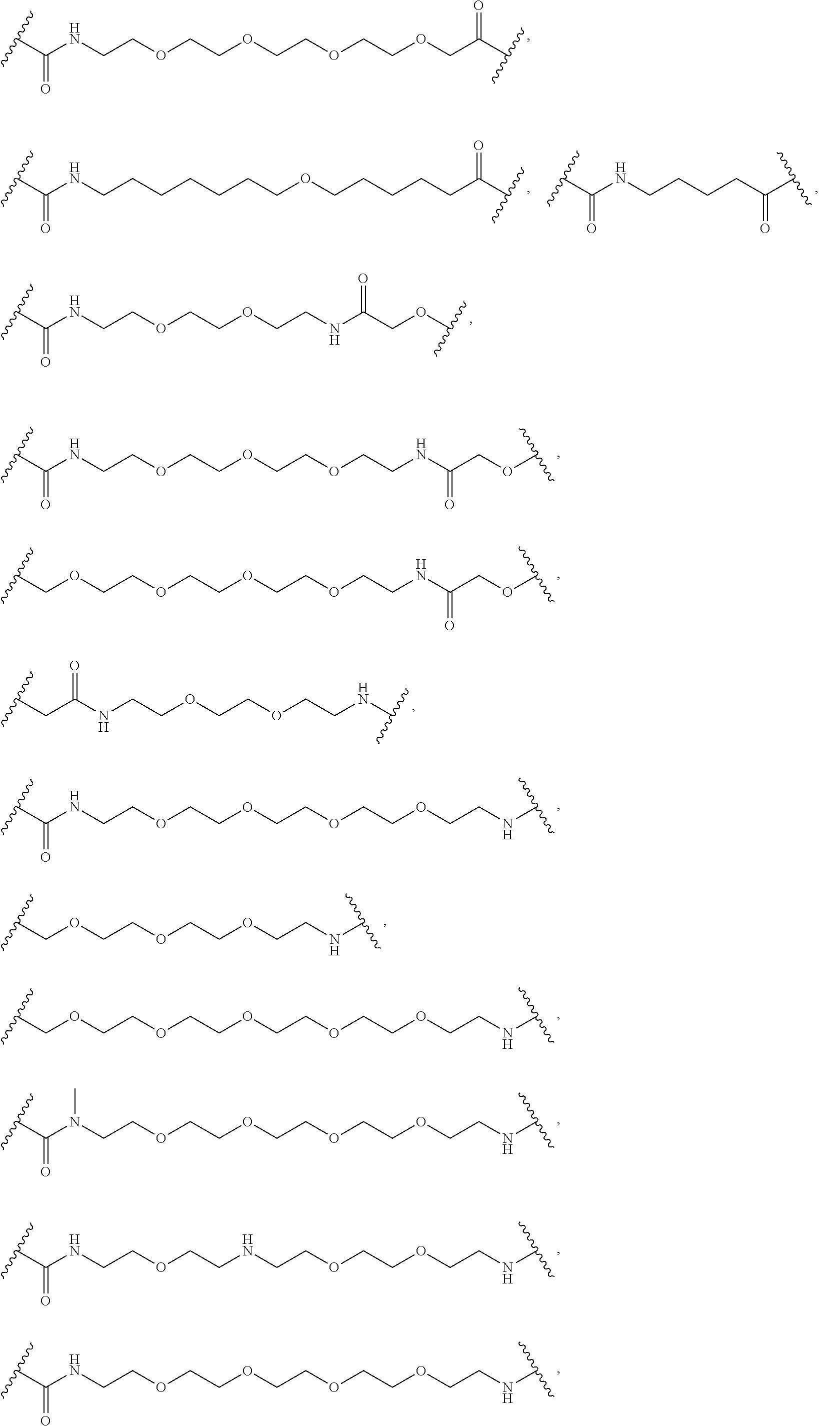

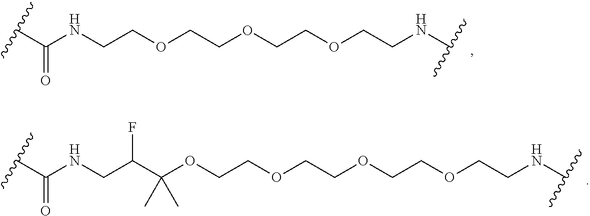 [SMILES: *NCCOCCOCCOCCNC(*)=O ; *NCCOCCOCCOCCOC(C)(C)C(F)CNC(*)=O]

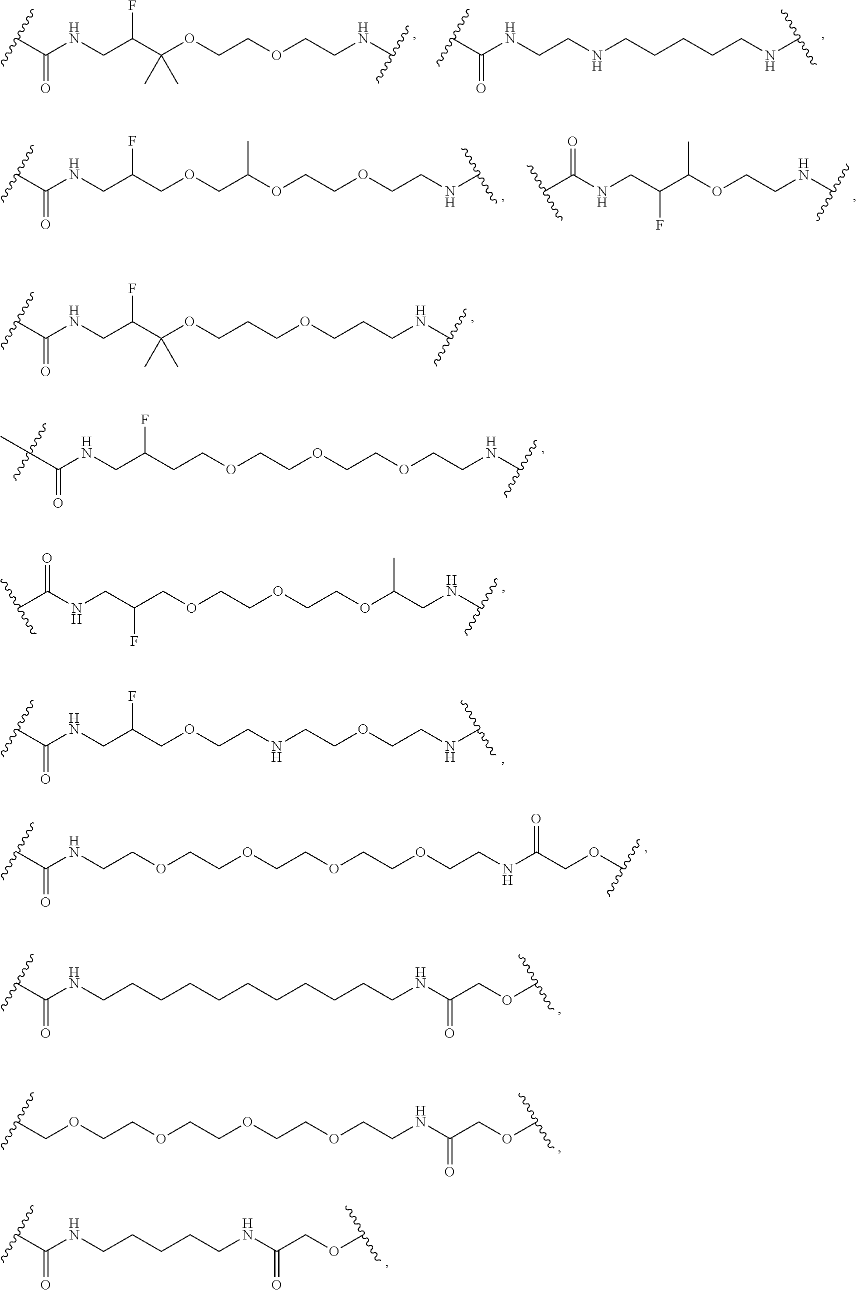

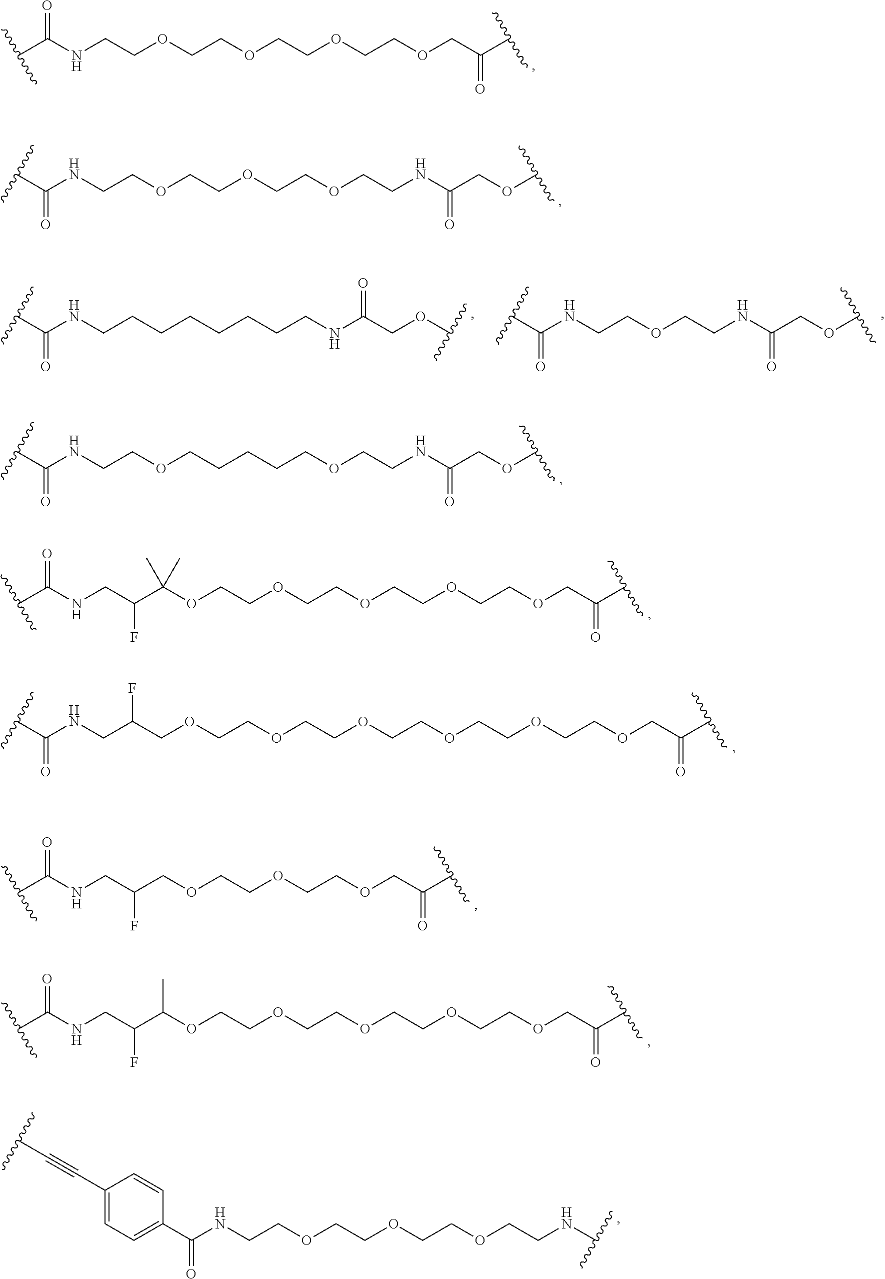

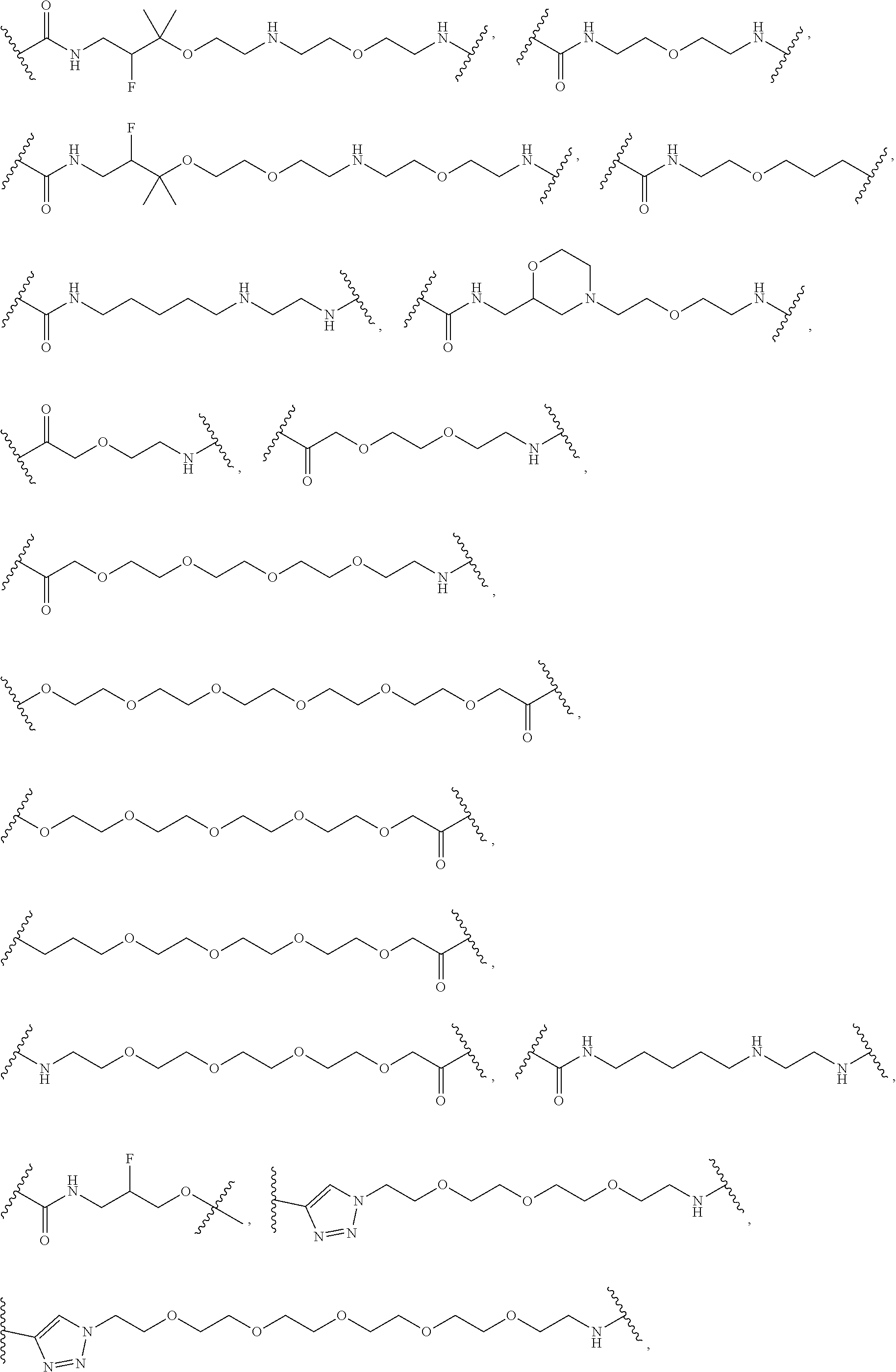

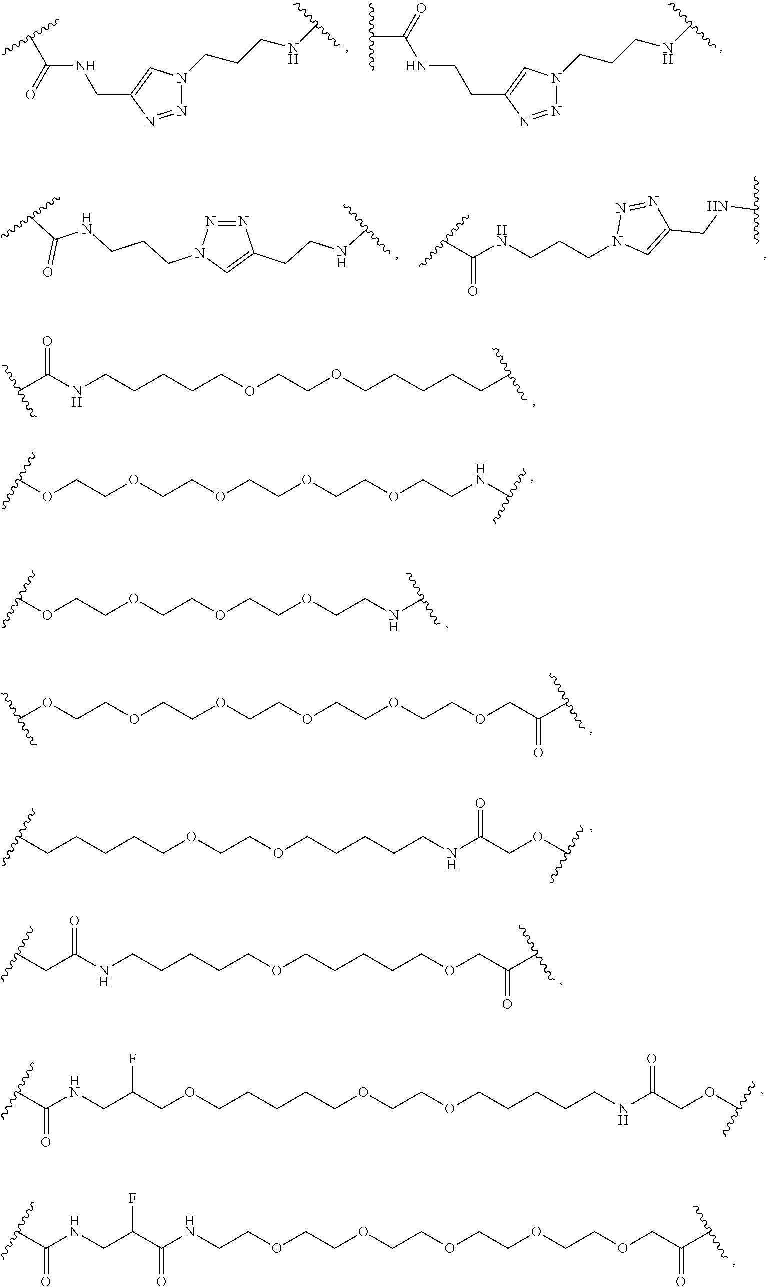

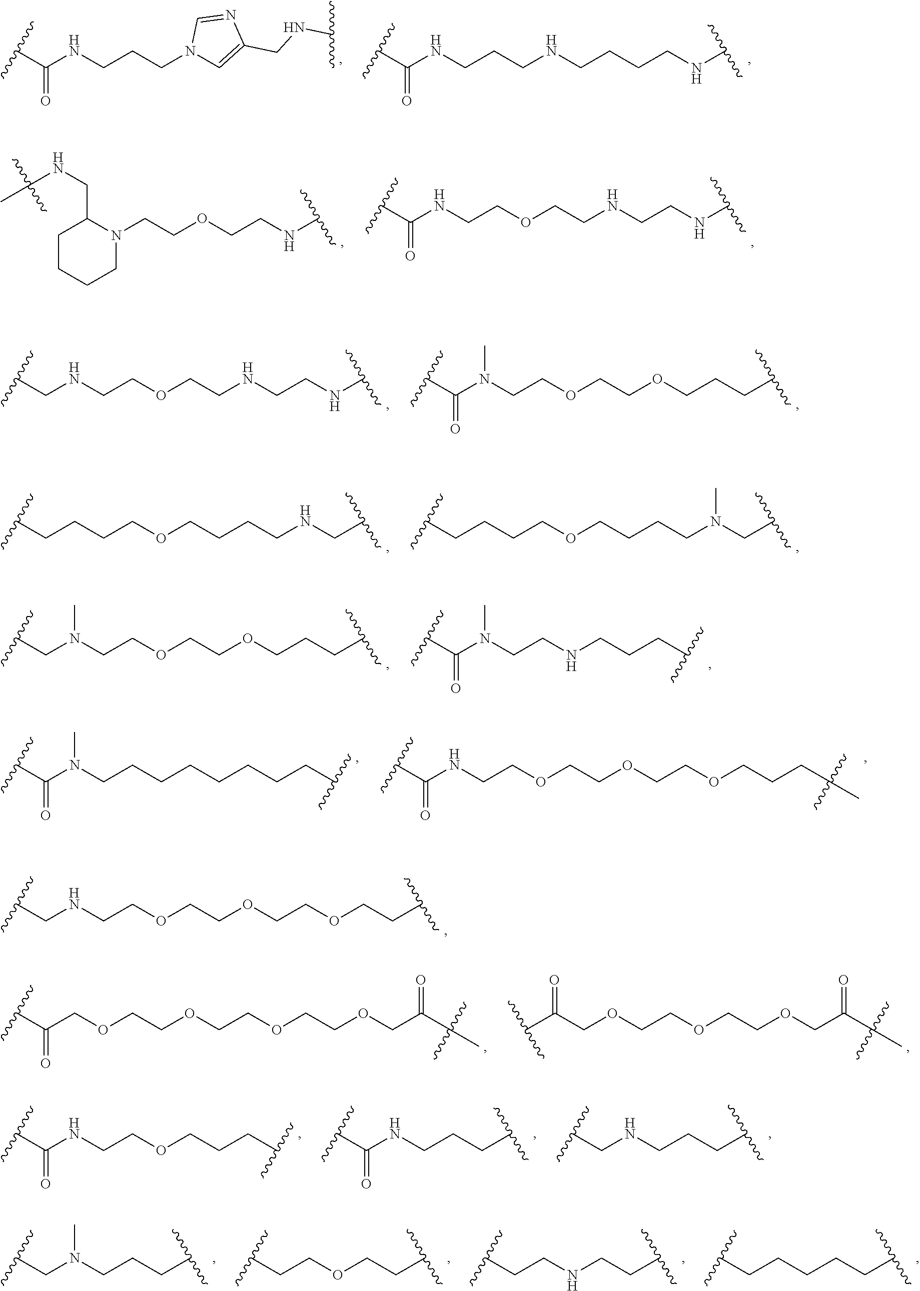

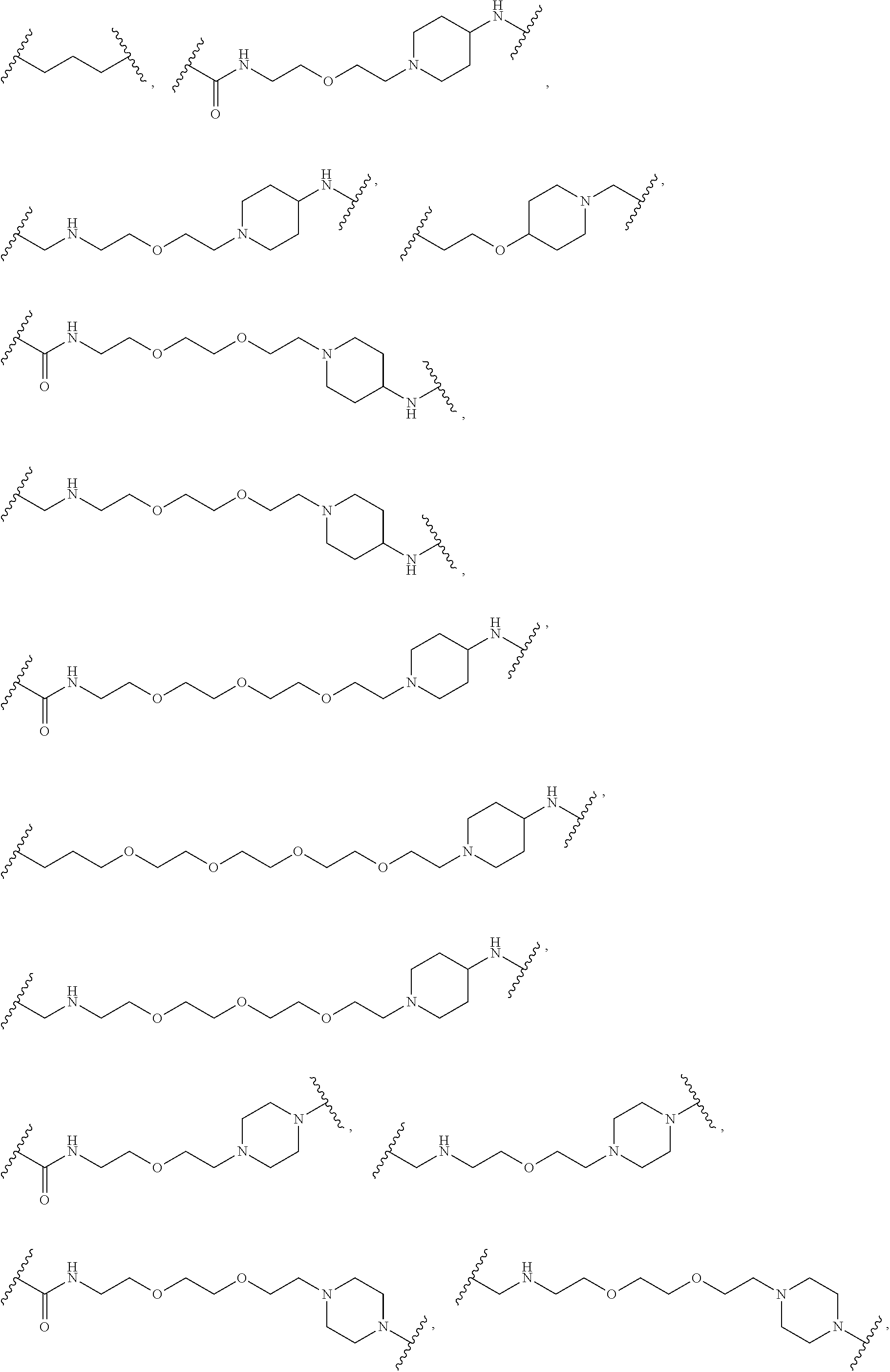

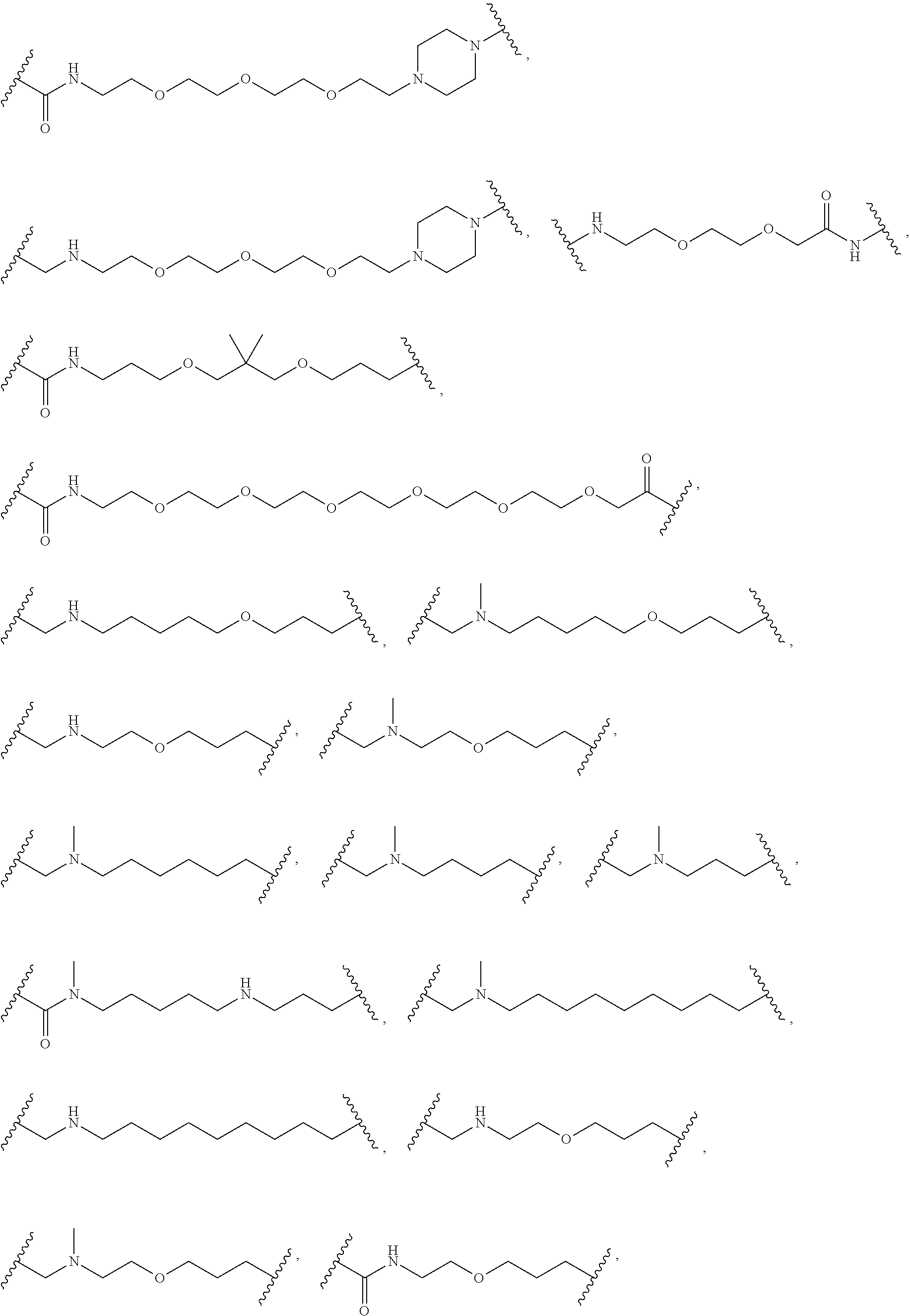

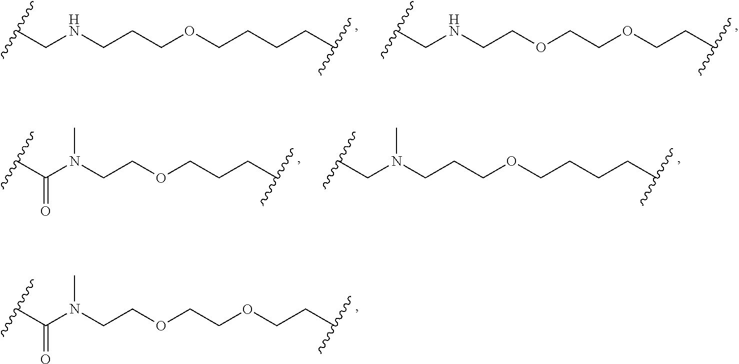 [SMILES: *CCCCOCCCNC* ; *CCOCCOCCNC* ; *CCCOCCN(C)C(*)=O ; *CCCCOCCCN(C)C* ; *CCOCCOCCN(C)C(*)=O]

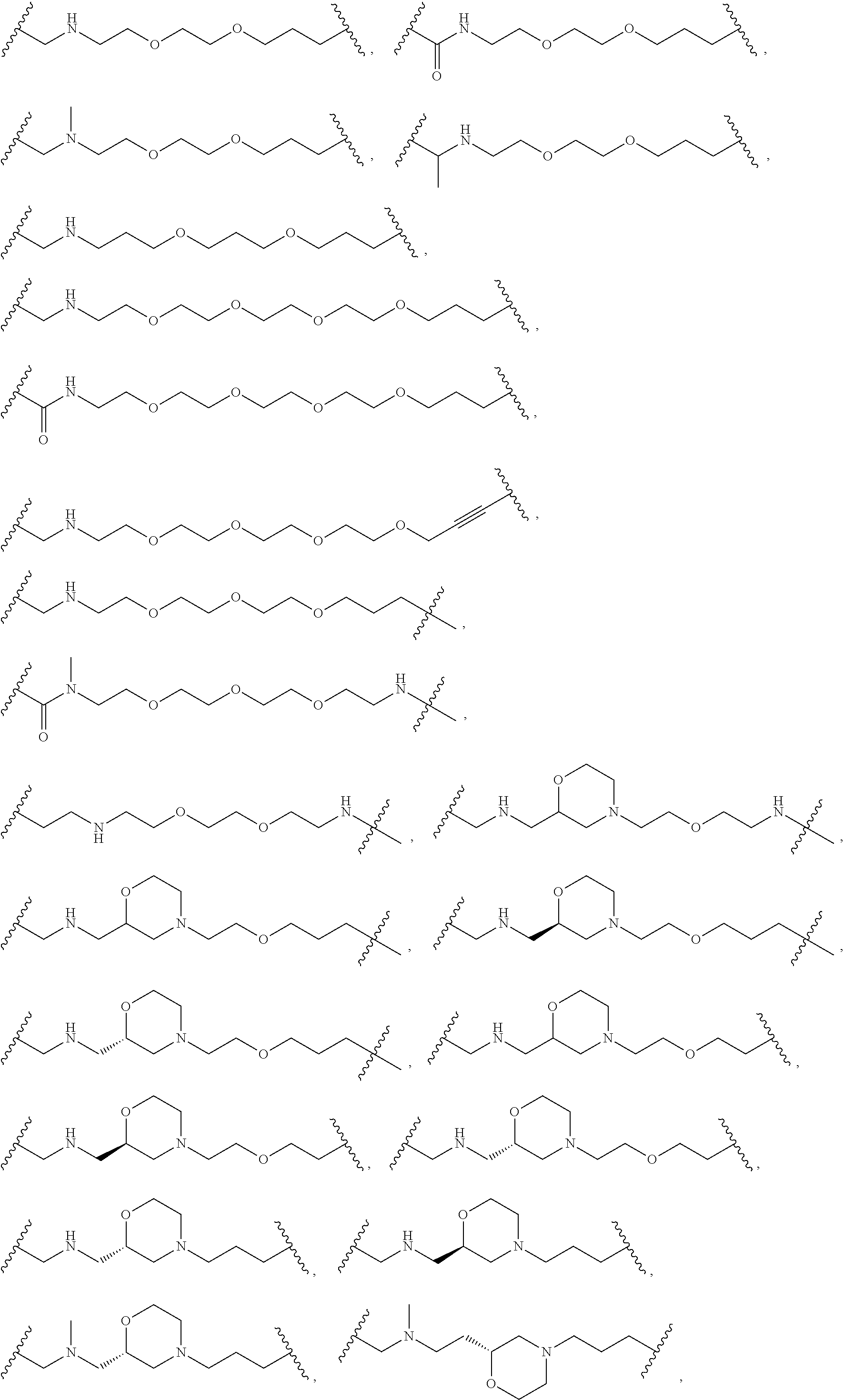

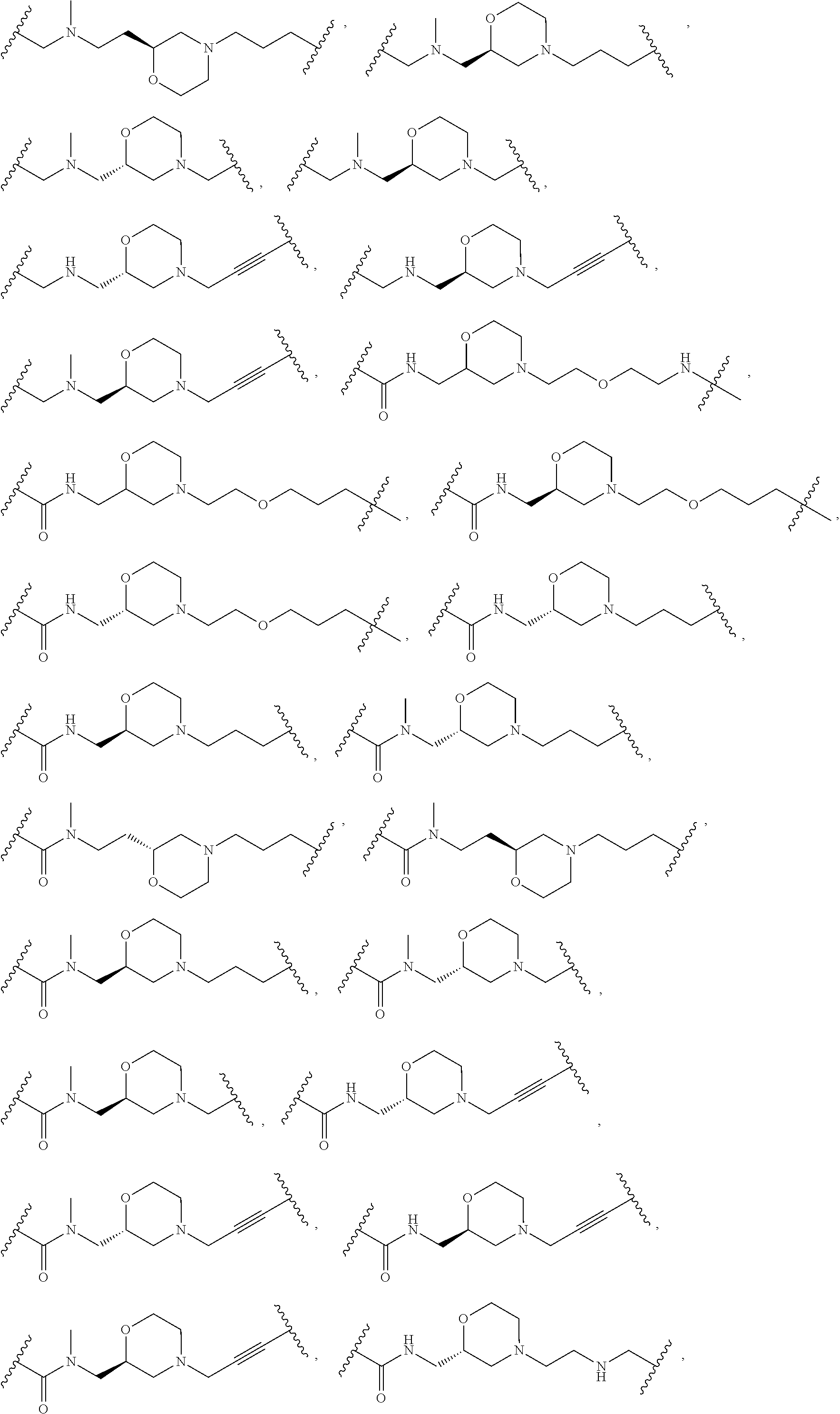

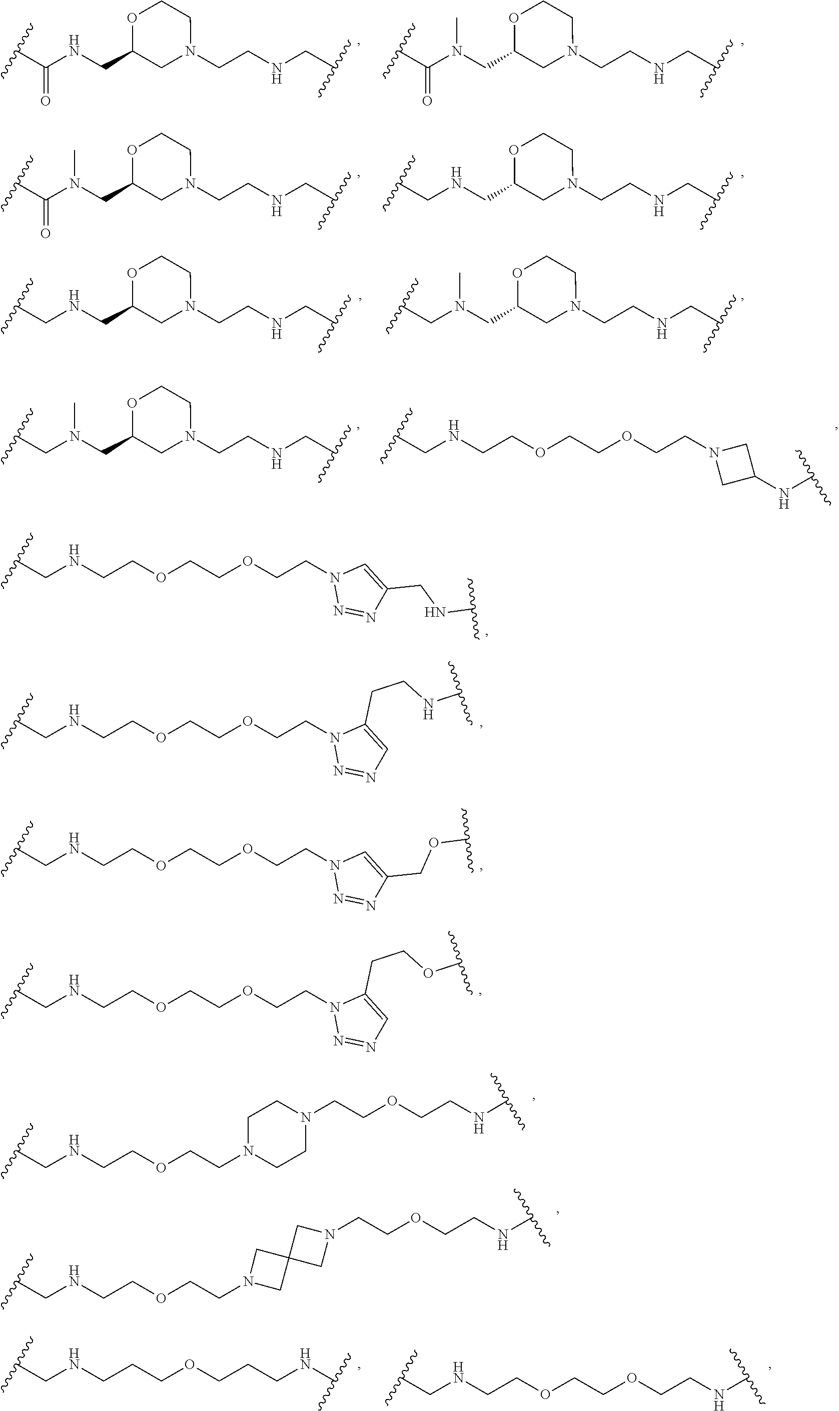

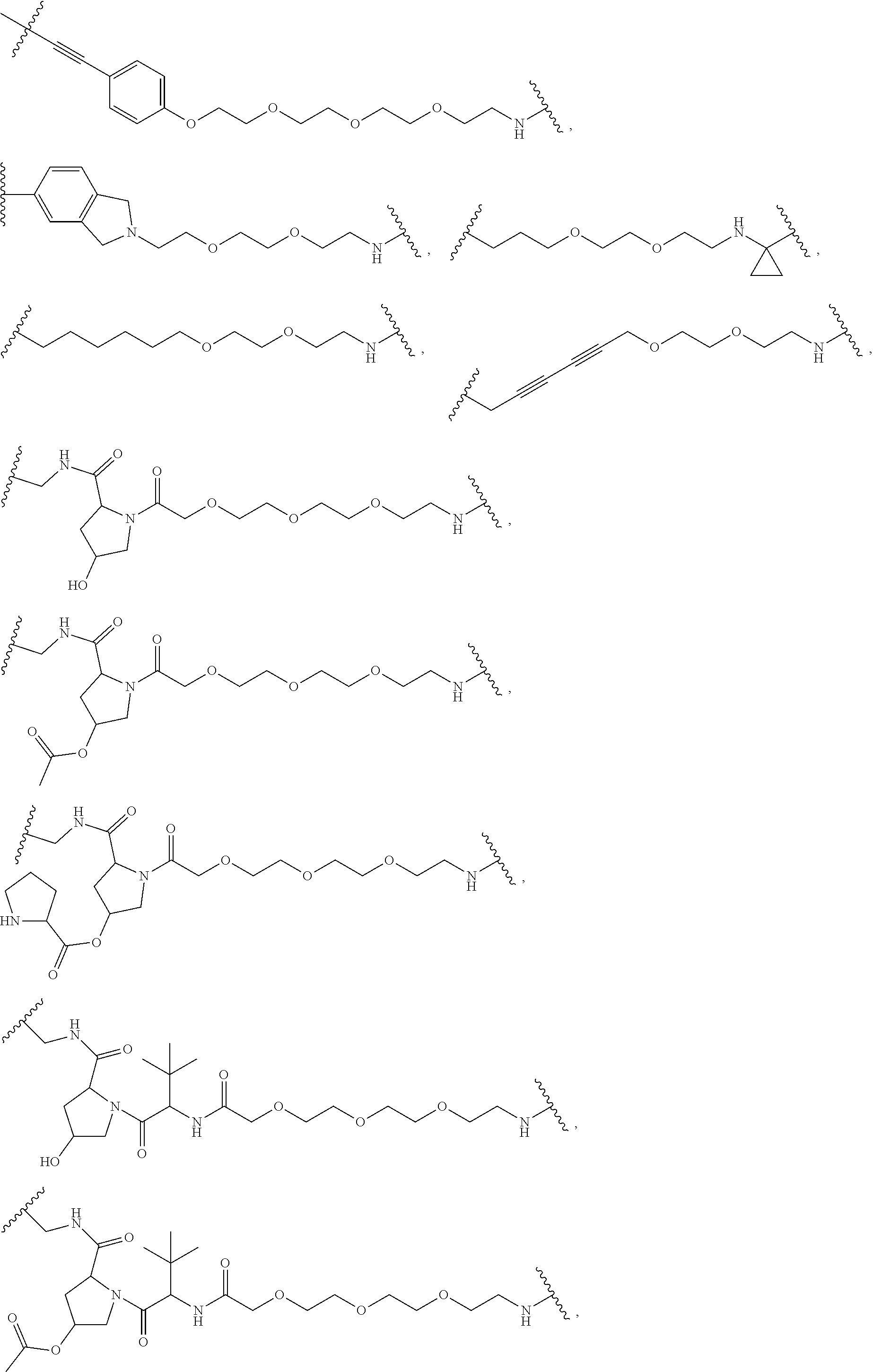

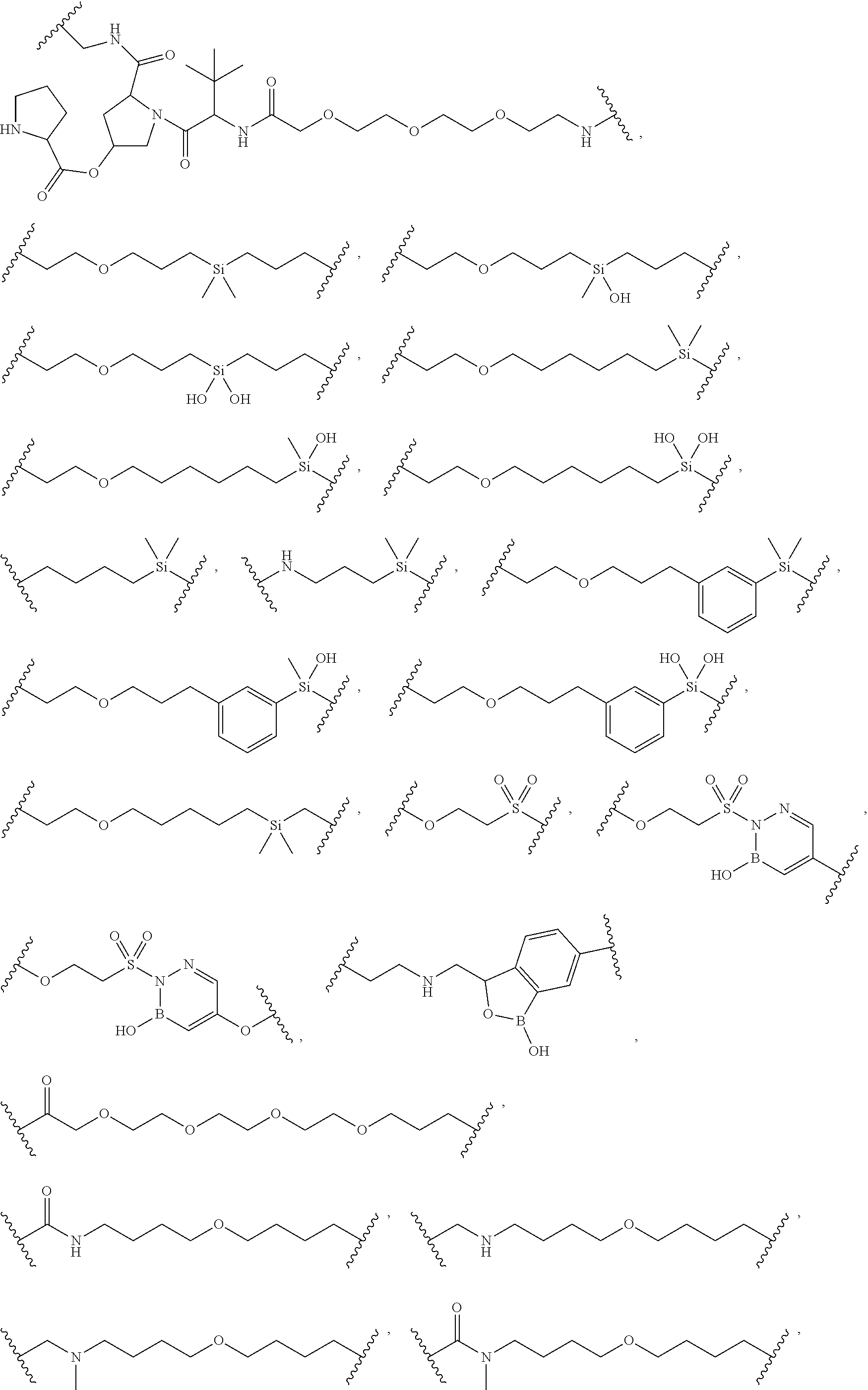

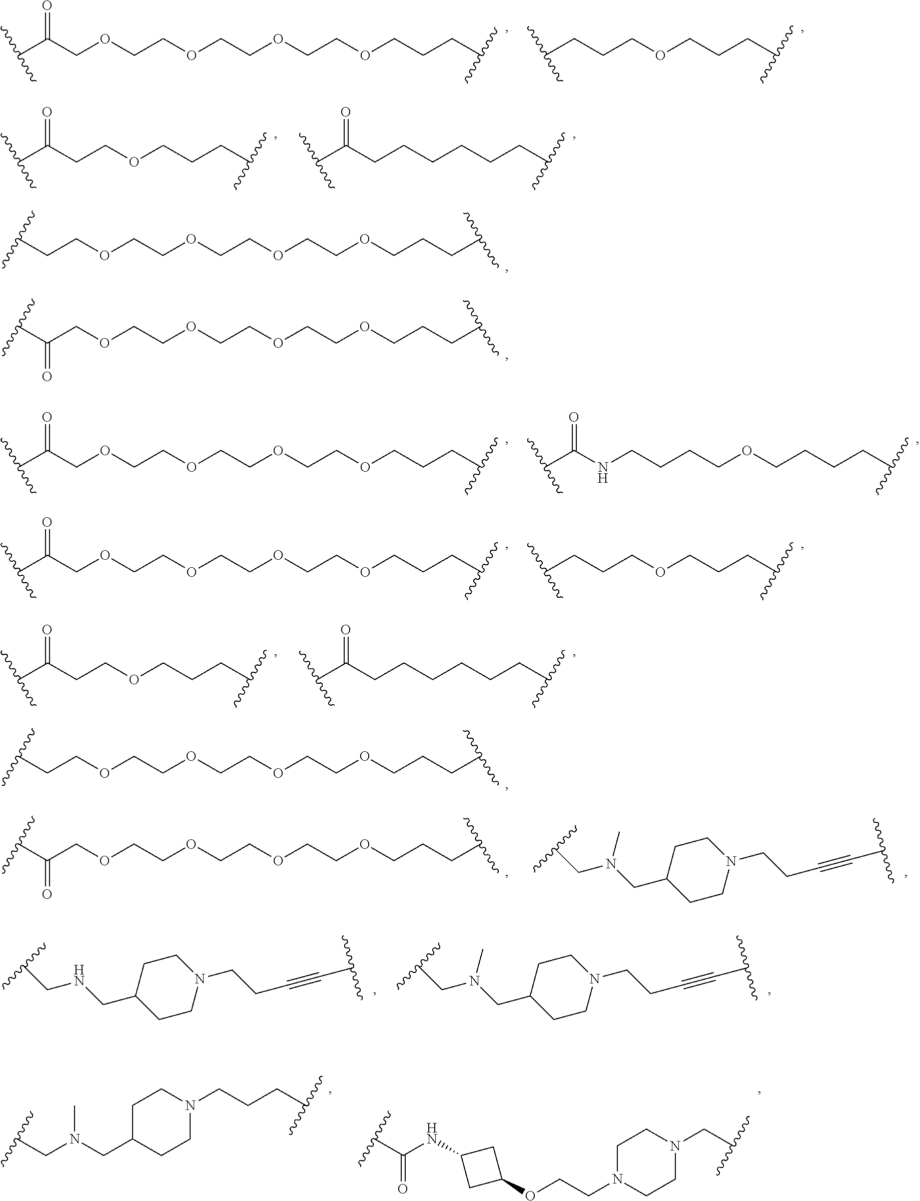

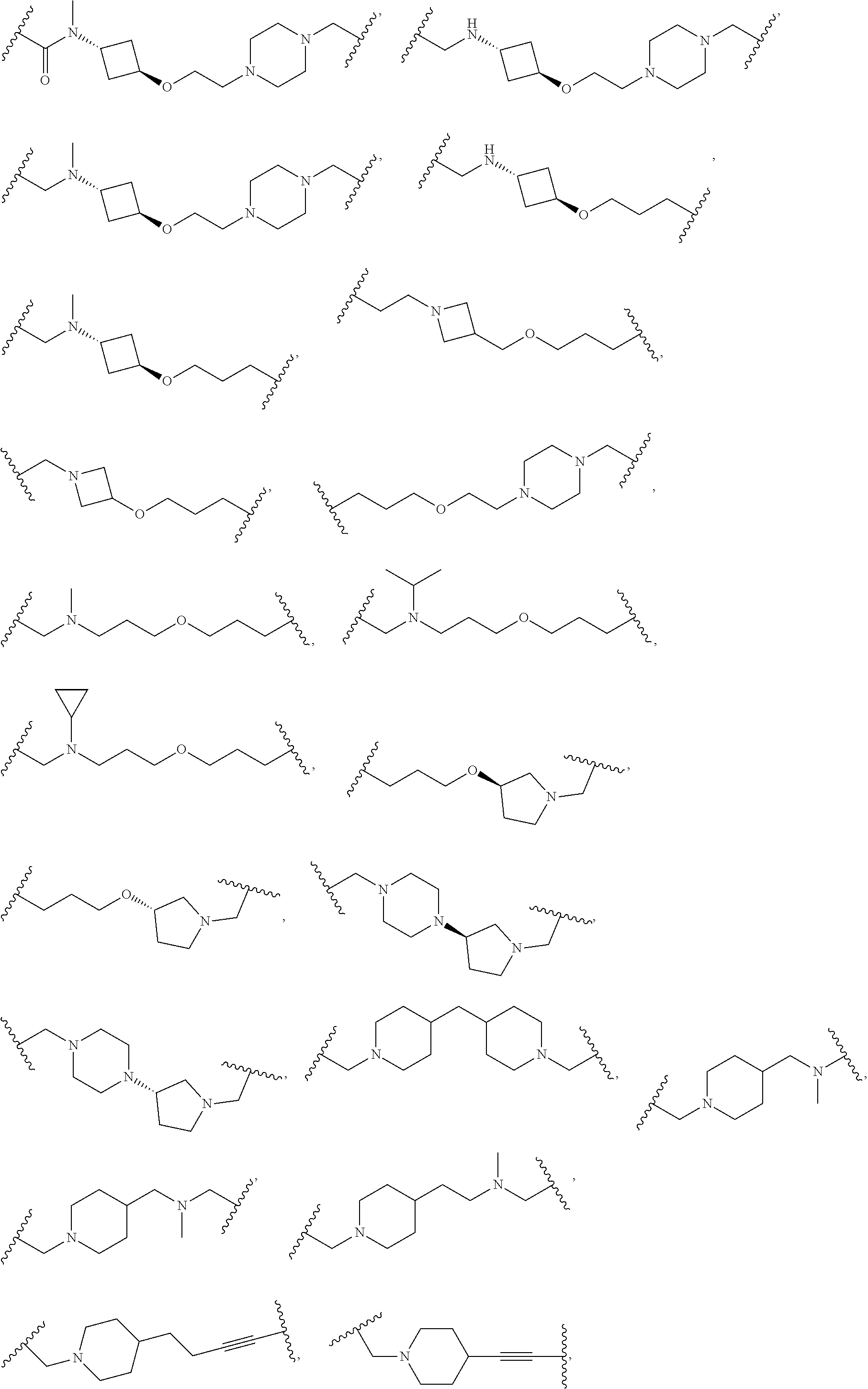

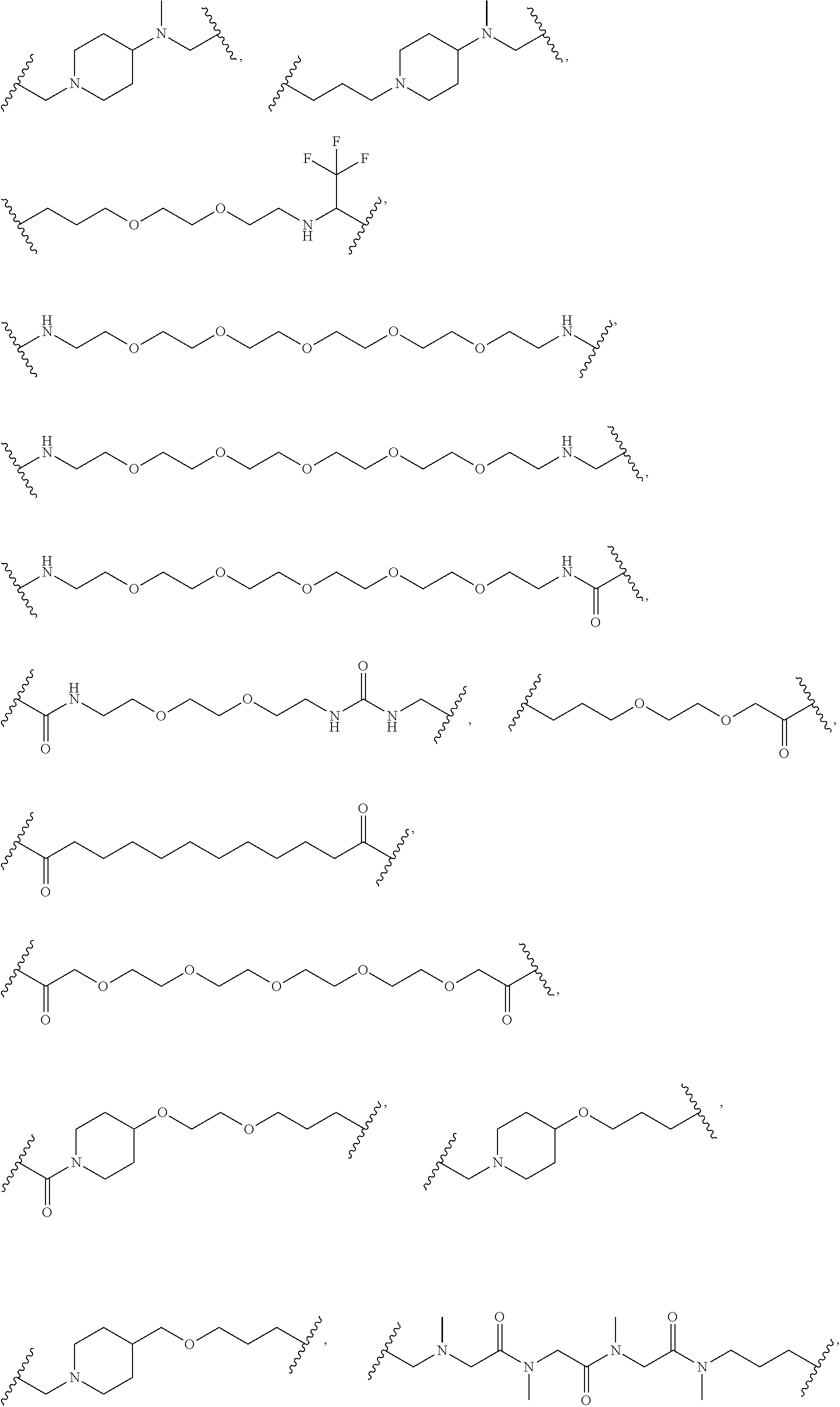

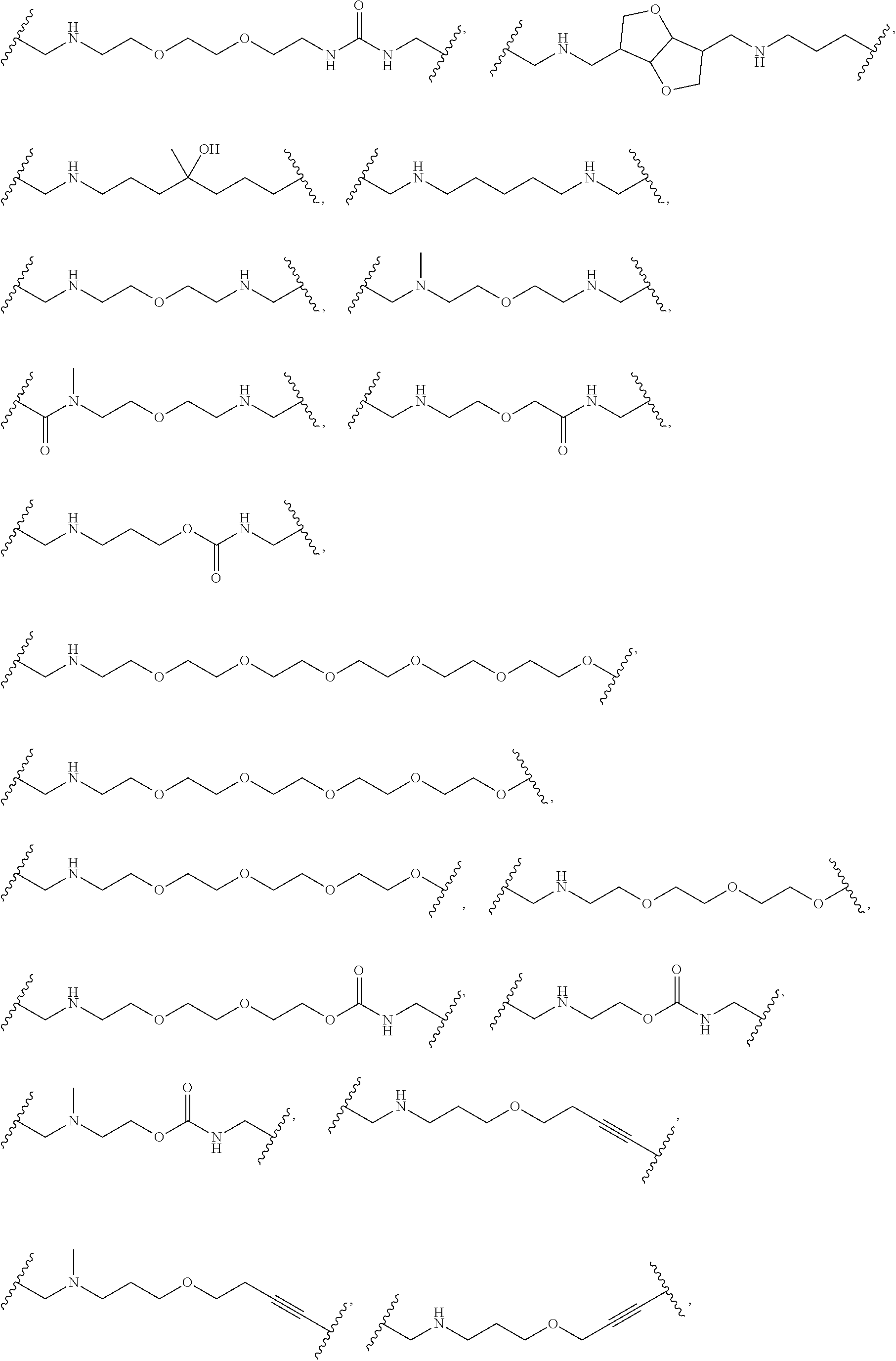

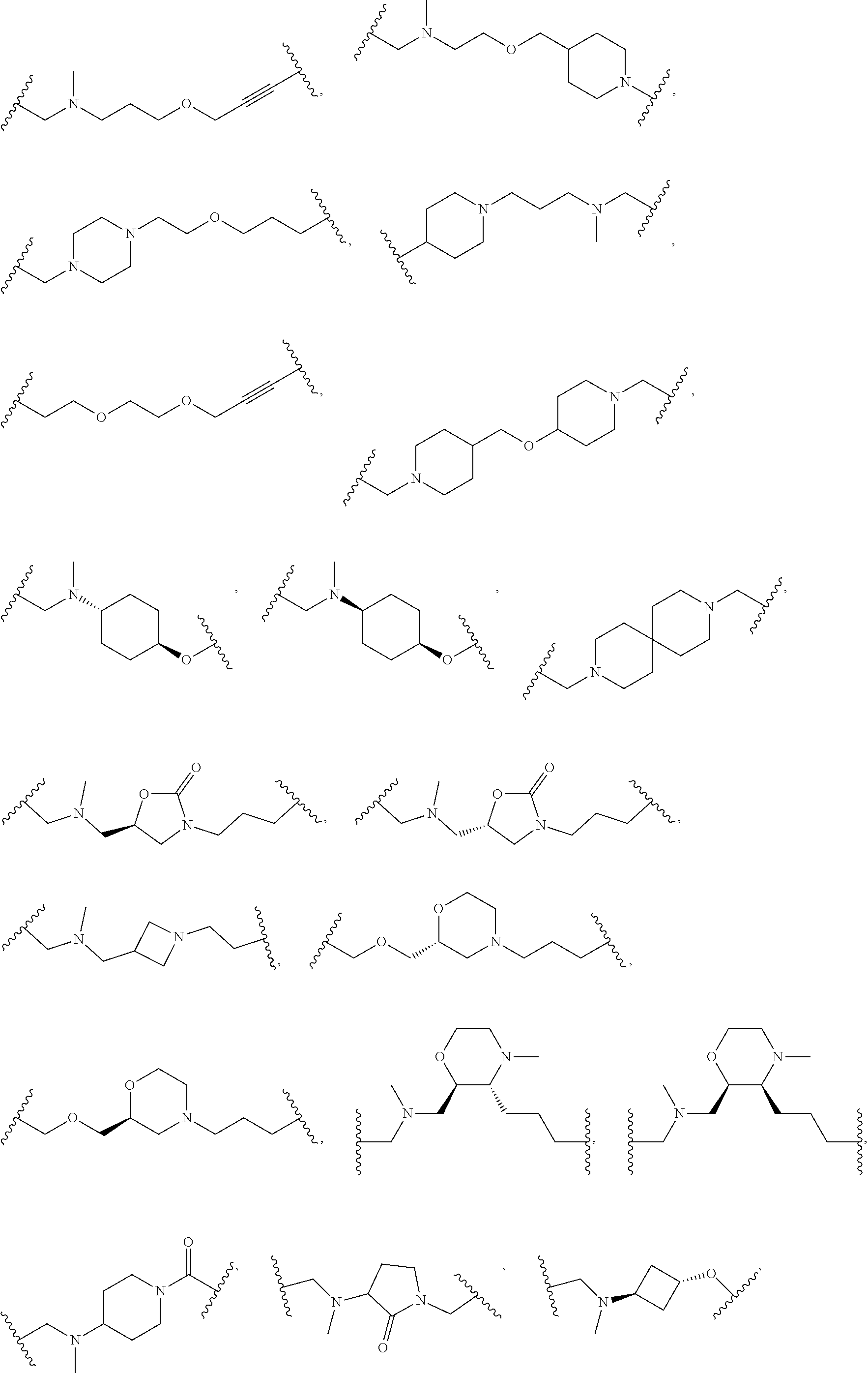

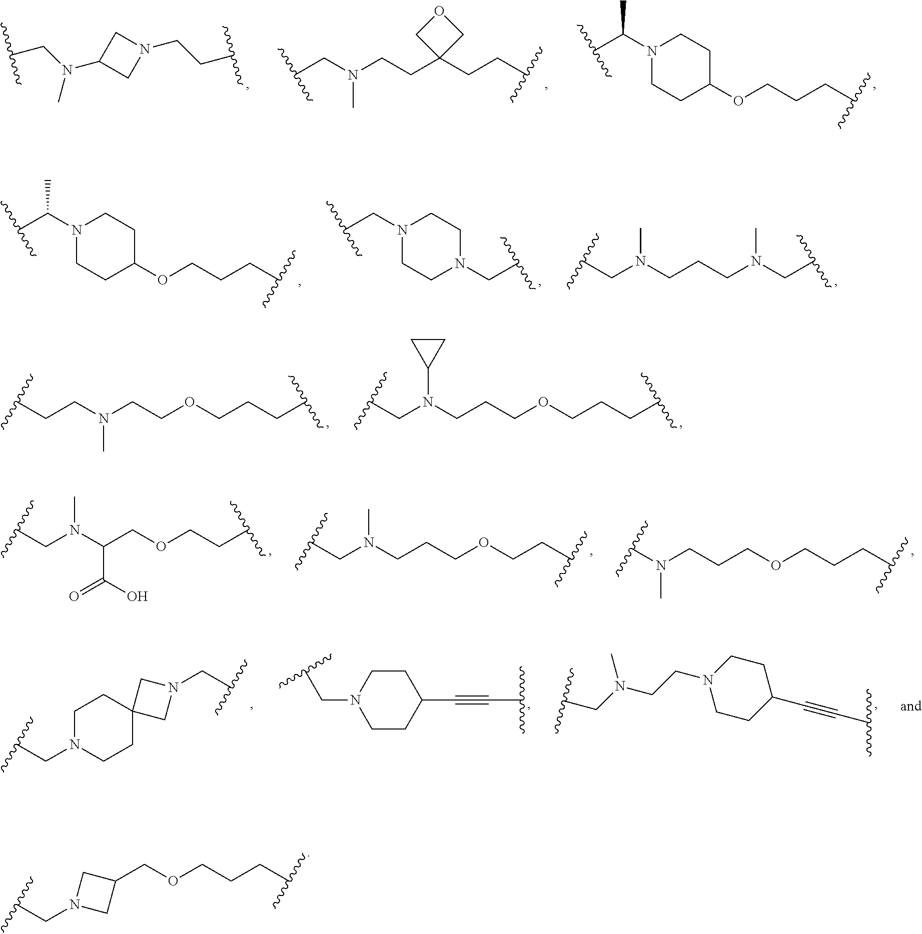

Descriptions of most of the linkers above are disclosed in published US patent application US 2019/0192668 A1.

### Embodiment 11

The bifunctional degrader of any one of Embodiments 1-10a, wherein the protein of interest is FKBP12.

### Embodiment 12

The bifunctional degrader of Embodiment 11, wherein ligand A is SLF, has the structure (A-1):

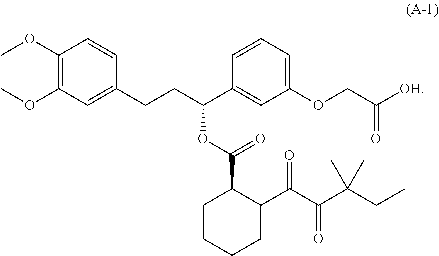 [SMILES: CCC(C)(C)C(=O)C(=O)C1CCCC[C@H]1C(=O)O[C@H](CCc1ccc(OC)c(OC)c1)c1cccc(OCC(=O)O)c1]

### Embodiment 13

The bifunctional degrader of either Embodiment 11 or 12, wherein the linker B is

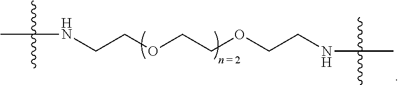

### Embodiment 14

The bifunctional degrader of any one of Embodiments 11-13, having the structure:

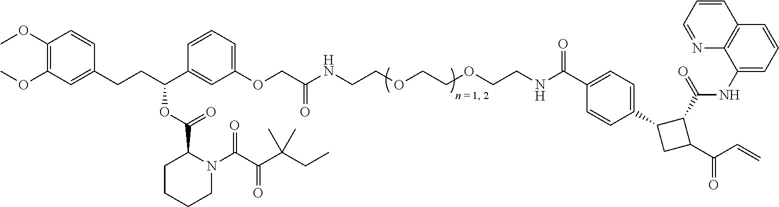

or a pharmaceutically acceptable salt thereof.

### Embodiment 15

The bifunctional degrader of any one of Embodiments 1-10, wherein the protein of interest is the androgen receptor.

### Embodiment 16

The bifunctional degrader of Embodiment 15, wherein ligand A comprises at least one chemical moiety selected from the group consisting of:

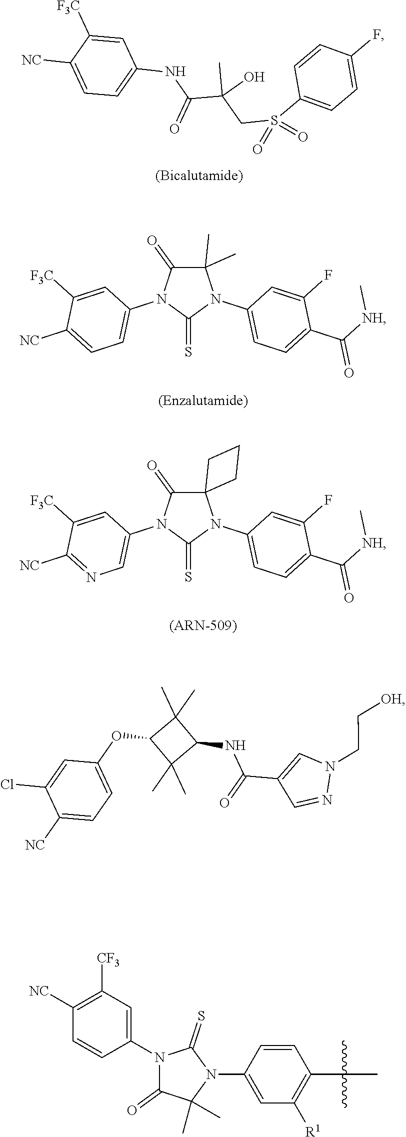

wherein
R¹ is F,

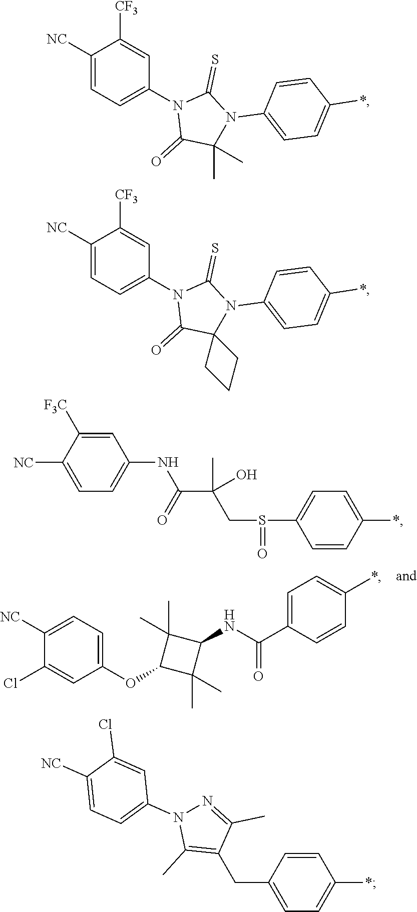

wherein in each instance, [symbol] or * can be a point of attachment for the linker B.

Various androgen receptor ligands are disclosed in:
Xin Han et al., J. Med. Chem. 2019, 62, 941-964; DOI: 10.1021/acs.jmedchem.8b01631; and Michael L. Mohler et al., Int. J. Mol. Sci. 2021, 22, 2124. https://doi.org/10.3390/ijms22042124;

### Embodiment 17

The bifunctional degrader of any one of Embodiments 1-10, wherein the protein of interest is the estrogen receptor.

### Embodiment 18

The bifunctional degrader of Embodiment 17, wherein ligand A comprises at least one chemical moiety selected from the group consisting of:

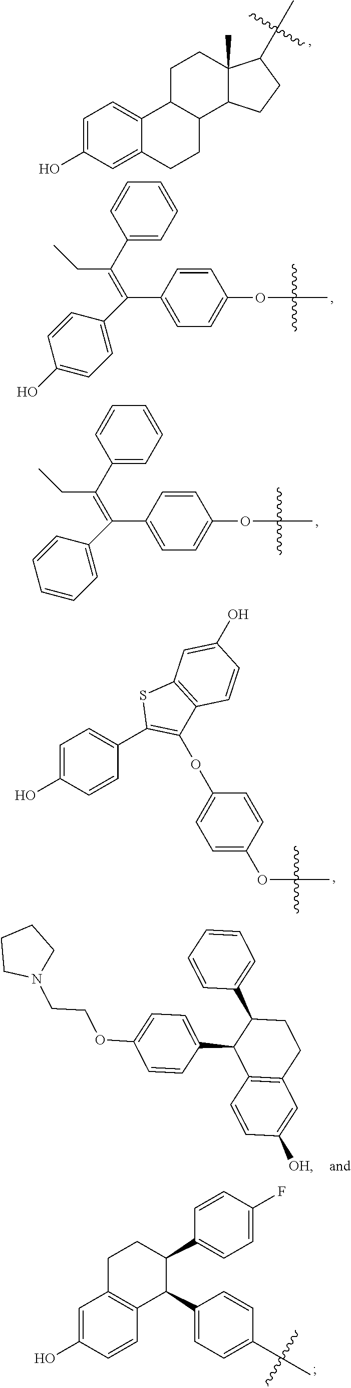

wherein [symbol] can be a point of attachment for the linker B.

Various ligands for the estrogen receptor are disclosed for example in the following references: Hui Qin et al., Curr Med Chem. 2021 Nov. 9. doi: 10.2174/0929867328666211110101018; and U.S. Pat. No. 10,071,164.

### Embodiment 19

The bifunctional degrader of any one of Embodiments 1-10, wherein the protein of interest is IRAK4.

### Embodiment 20

The bifunctional degrader of Embodiment 19, wherein ligand A comprises at least one chemical moiety selected from the group consisting of:

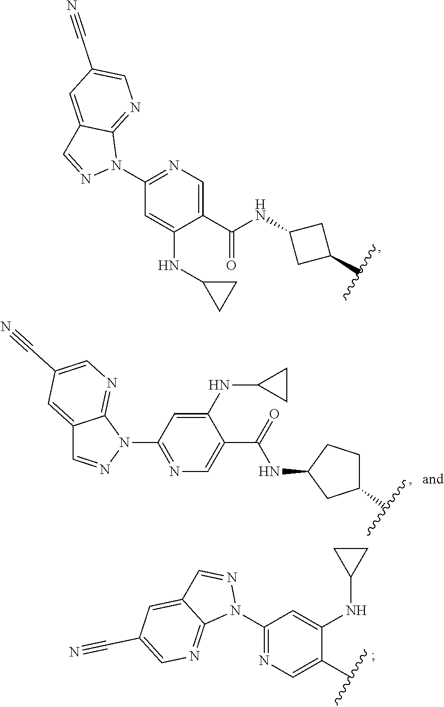

wherein [symbol] can be a point of attachment for the linker B.

Various ligands for IRAK are disclosed for example, in published US patent application US 2019/0192668 A1.

### Embodiment 21

The bifunctional degrader of any one of Embodiments 1-10, wherein the protein of interest is a JAK protein.

### Embodiment 22

The bifunctional degrader of Embodiment 21, wherein the JAK protein is JAK1, JAK2, or JAK3.

### Embodiment 23

The bifunctional degrader of Embodiment 21 or 22, wherein ligand A comprises at least one chemical moiety selected from the group consisting of:

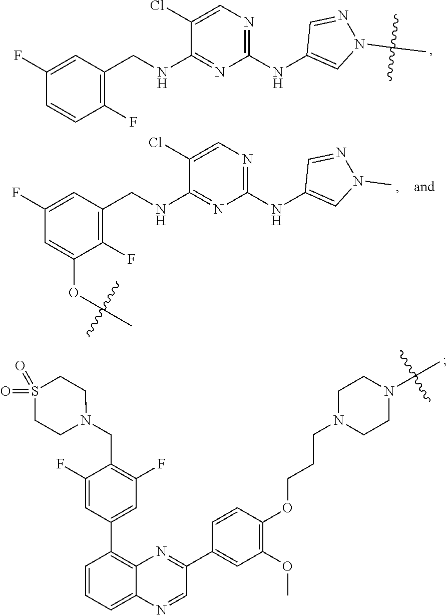

wherein [symbol] can be a point of attachment for the linker B.

Ligands for various JAK proteins are disclosed in Rishi R. Shah et al., Bioorganic & Medicinal Chemistry, Volume 28, Issue 5, 1 Mar. 2020, 115326 (including the supplemental supporting information); https://doi.org/10.1016/j.bmc.2020.115326.

### Embodiment 24

The bifunctional degrader of any one of Embodiments 1-10, wherein the protein of interest is BCL-XL or BCL-2.

### Embodiment 25

The bifunctional degrader of Embodiment 24, wherein ligand A comprises at least one chemical moiety selected from the group consisting of:

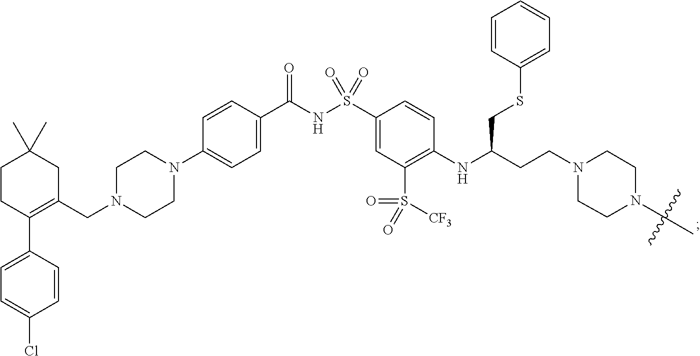 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

wherein [symbol] can be a point of attachment for the linker B.

Various ligands for BCL-XL and BCL-2 are disclosed in Sajid Khan et.al., Nature Medicine, VOL 25, December 2019, 1938-1947, and www.nature.com/articles/s41591-019-0668-z.pdf; doi: 10.1038/s41591-019-0668-z.

### Embodiment 26

The bifunctional degrader of any one of Embodiments 1-10, wherein the protein of interest is Stat3.

### Embodiment 27

The bifunctional degrader of Embodiment 26, wherein ligand A comprises at least one chemical moiety of Formula (A-2):

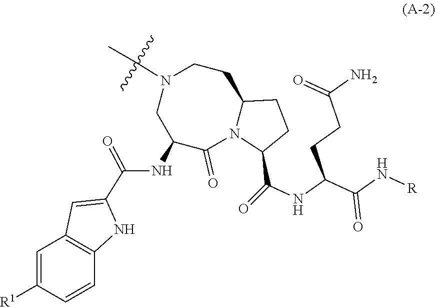

wherein:
R is:

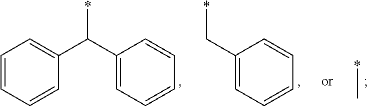

and
R¹ is:

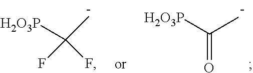

wherein * is the point of attachment for R and R¹, and [symbol] can be a point of attachment for the linker B.

Various ligands for Stat3 protein are disclosed, for example, in the following references:
Longchuan Bai et al., Cancer Cell 36, 498-511, Nov. 11, 2019;
https://doi.org/10.1016/j.ccell.2019.10.002;
Haibin Zhou et al., ACS Medicinal Chemistry Letters, 2021 May 10; 12(6):996-1004;
https://doi.org/10.1021/acsmedchemlett.1c00155; and
Jianyong Chen et al., ACS Med Chem Lett., 2010 May 13; 1(2):85-89. doi: 10.1021/ml100010j.

### Embodiment 28

The bifunctional degrader of any one of Embodiments 1-10, wherein the protein of interest is BRD4.

### Embodiment 29

The bifunctional degrader of Embodiment 28, wherein ligand A comprises at least one chemical moiety selected from the group consisting of:

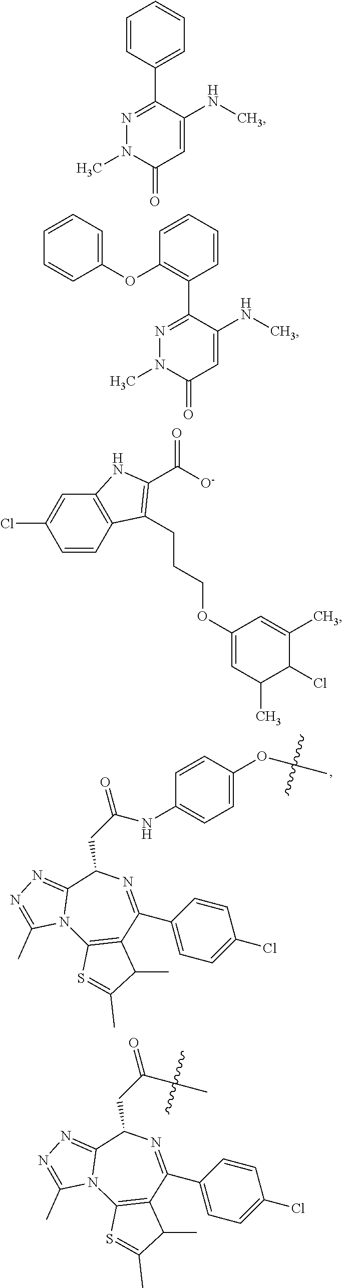

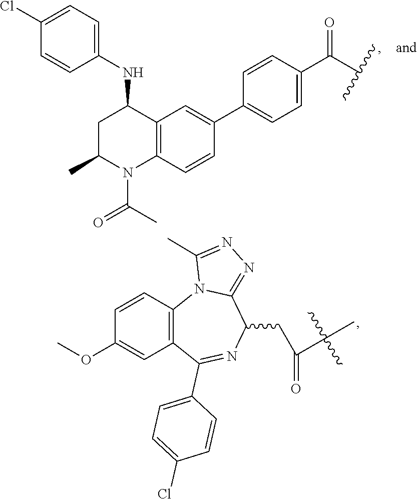

wherein [symbol] can be a point of attachment for the linker B.

Various ligands for BRD4 are disclosed for example, in the following references:
Germano Heinzelmann & Michael K. Gilson, Scientifc Reports, (2021) 11:1116; doi: 10.1038/s41598-020-80769-1;
Bryce K. Allen et al., ACS Omega 2017, 2, 4760-4771, doi: 10.1021/acsomega.7600553;
Tamara Minko, Trends Pharmacol Sci. 2020 October; 41(10):684-686. doi: 10.1016/j.tips.2020.08.008;
Serena G. Piticchio et al., J. Med. Chem. 2021, 64, 17887-17900; doi:
10.1021/acs.jmedchem. 1c01108m; including supporting information at
https://pubs.acs.org/doi/10.1021/acs.jmedchem.1c01108;
Fred L. Ciske and Thomas G. Brock, Ph.D., Cayman Chemical. Bromodomain Targeting with PROTACs. Article from 2017 Apr. 5;
https://www.caymanchem.com/news/bromodomain-targeting-with-protacs; and
U.S. Pat. No. 11,179,373 B2

### Embodiment 30

The bifunctional degrader of Embodiment 28 or 29, wherein ligand A is

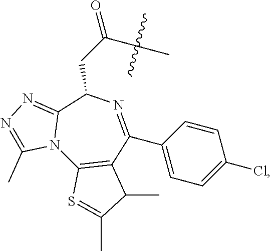

and the linker B is

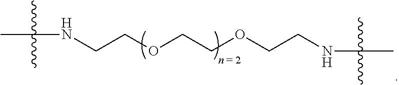

### Embodiment 31

The bifunctional degrader of Embodiment 30, having the structure:

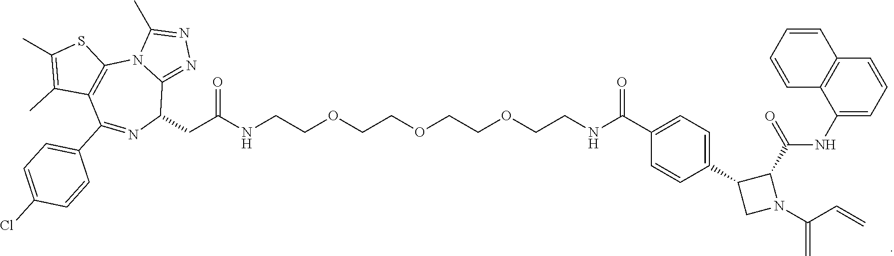 [SMILES: C=CC(=C)N1C[C@H](c2ccc(C(=O)NCCOCCOCCOCCNC(=O)C[C@@H]3N=C(c4ccc(Cl)cc4)c4c(sc(C)c4C)-n4c(C)nnc43)cc2)[C@@H]1C(=O)Nc1cccc2ccccc12]

### Embodiment 32

A pharmaceutical composition comprising the bifunctional degrader of any one of Embodiments 1-31 or a pharmaceutically acceptable salt thereof, and a pharmaceutically acceptable carrier.

### Embodiment 33

The pharmaceutical composition of Embodiment 32, further comprising an additional therapeutic agent.

### Embodiment 34

A method of degrading a protein selected from the group consisting of FKBP12, BRD4, androgen receptor, estrogen receptor, IRAK4, a JAK protein, BCL-XL, BCL-2, and Stat3 in a patient or biological sample comprising administering to said patient, or contacting said biological sample with the bifunctional degrader of any one of Embodiments 1-31.

### Embodiment 35

A method of treating a disorder, disease or condition mediated by a protein selected from the group consisting of FKBP12, BRD4, androgen receptor, estrogen receptor, IRAK4, a JAK protein, BCL-XL, BCL-2, and Stat3, in a patient, comprising administering to the patient a therapeutically effective amount of the degrader of any of Embodiments 1-31 or a pharmaceutically acceptable salt thereof.

### Embodiment 36

The method of Embodiment 35, wherein the disorder, disease or condition is a cancer, a neurodegenerative disease, a viral disease, an autoimmune disease, an inflammatory disorder, a hereditary disorder, a hormone-related disease, a hematopoietic disorder, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, and a CNS disorder.

### Embodiment 37

The method of either Embodiment 35 or 36, further comprising administering an additional therapeutic agent.

### Embodiment 38

A DCAF1 protein-probe adduct, wherein the probe binds to cysteine residue C1113 of DCAF1, wherein the amino acid numbering is based on DCAF1 Isoform 1 (Accession No. Q9Y4B6-1), cysteine residue C1112 of DCAF1, wherein the amino acid numbering is based on DCAF1 Isoform 2 (Accession No. Q9Y4B6-2), or cysteine residue C664 of DCAF1, wherein the amino acid numbering is based on DCAF1 Isoform 3 (Accession No. Q9Y4B6-3) and wherein the probe comprises an azetidinyl acrylamide moiety.

### Embodiment 39

The DCAF1 protein-probe adduct of Embodiment 38, wherein the probe is a compound of Formula (I):

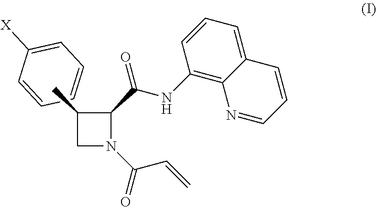

wherein:

X is selected from the group consisting of halo,

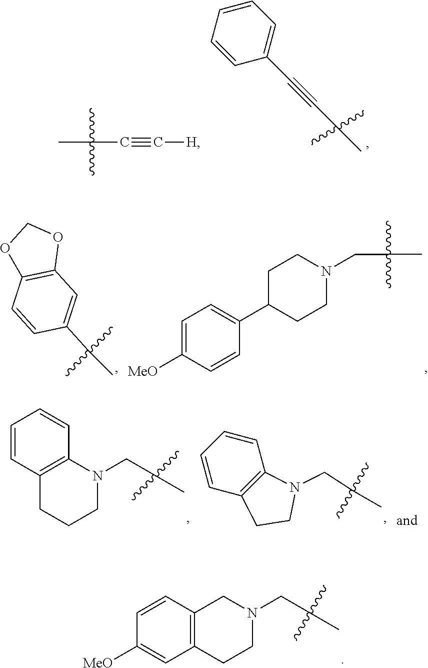

### Embodiment 40

The DCAF1 protein-probe adduct of Embodiment 39, having the structure of Formula (II):

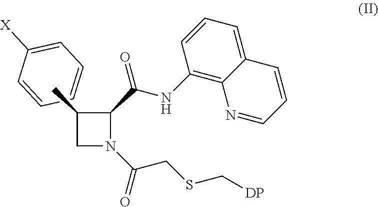

wherein:

S represents the sulfur atom of a cysteine residue C1113 of DCAF1, wherein the amino acid numbering is based on DCAF1 Isoform 1 (Accession No. Q9Y4B6-1), cysteine residue C1112 of DCAF1, wherein the amino acid numbering is based on DCAF1 Isoform 2 (Accession No. Q9Y4B6-2), or cysteine residue C664 of DCAF1, wherein the amino acid numbering is based on DCAF1 Isoform 3 (Accession No. Q9Y4B6-3); and

DP represents the DCAF1 polypeptide wherein the amino acid numbering is based on DCAF1 Isoform 1 (Accession No. Q9Y4B6-1), DCAF1 Isoform 2 (Accession No. Q9Y4B6-2), or DCAF1 Isoform 3 (Accession No. Q9Y4B6-3).

### Embodiment 41

The DCAF1 protein-probe adduct of any of Embodiments 38-40, wherein the probe is selected from the group consisting of:

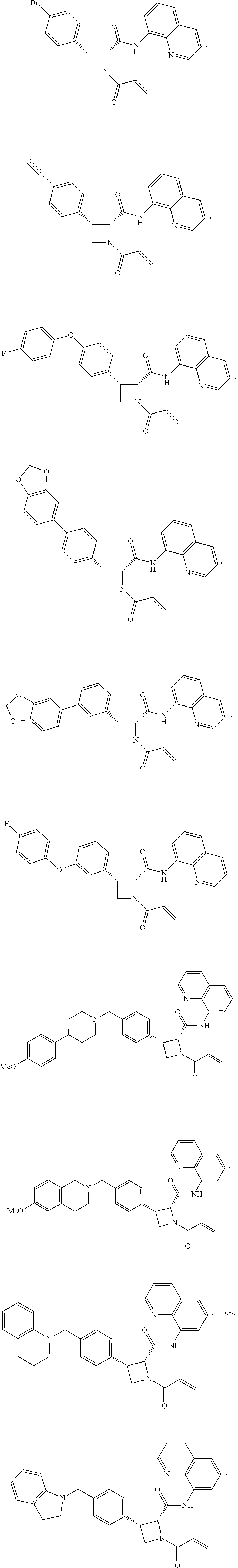

### Embodiment 42

A compound of Formula (I)

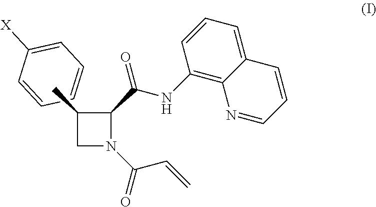

or a pharmaceutically acceptable salt thereof, wherein:

X is selected from the group consisting of halo,

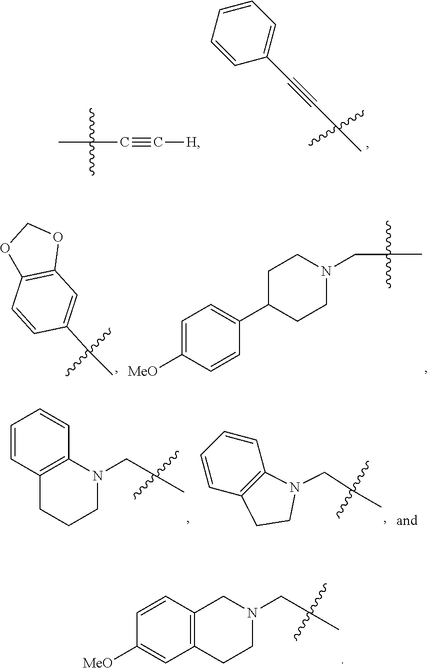

### Embodiment 43

The compound of Embodiment 42, selected from the group consisting of:

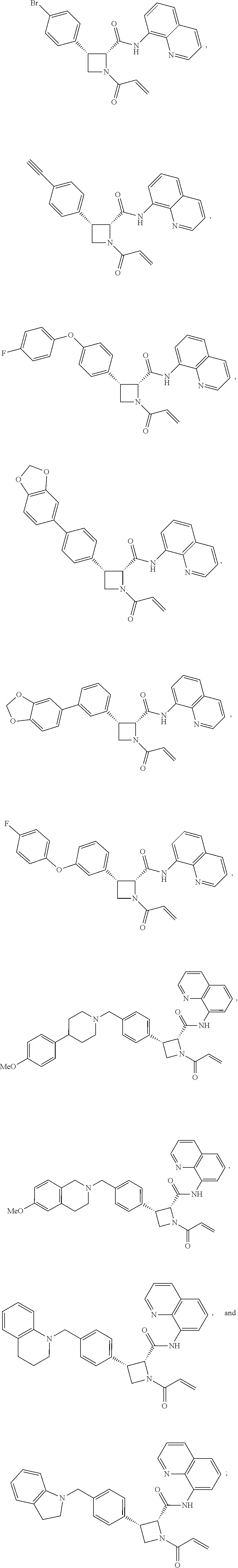

or a pharmaceutically acceptable salt thereof.

### Embodiment 44

A method of agonizing or antagonizing DCAF1 protein, wherein the amino acid numbering is based on DCAF1 Isoform 1 (Accession No. Q9Y4B6-1), in a patient in need of such agonization or antagonization, or in a biological sample, comprising administering to the patient, or contacting the biological sample with the compound of Embodiment 43 or 43, or a pharmaceutically acceptable salt thereof.

### EXAMPLES

Three isoforms of DCAF1 protein sequences.

### Isoform 1: (Identifier: Q9Y4B6-1)

```
                10 20 30 40 50
MTTVVVHVDS KAELTTLLEQ WEKEHGSGQD MVPILTRMSQ LIEKETEEYR
                60 70 80 90 100
KGDPDPFDDR HPGRADPECM LGHLLRILFK NDDFMNALVN AYVMTSREPP
              110 120 130 140 150
LNTAACRLLL DIMPGLETAV VFQEKEGIVE NLFKWAREAD QPLRTYSTGL
              160 170 180 190 200
LGGAMENQDI AANYRDENSQ LVAIVLRRLR ELQLQEVALR QENKRPSPRK
              210 220 230 240 250
LSSEPLLPLD EEAVDMDYGD MAVDVVDGDQ EEASGDMEIS FHLDSGHKTS
              260 270 280 290 300
SRVNSTTKPE DGGLKKNKSA KQGDRENFRK AKQKLGFSSS DPDRMFVELS
              310 320 330 340 350
NSSWSEMSPW VIGTNYTLYP MTPAIEQRLI LQYLTPLGEY QELLPIFMQL
              360 370 380 390 400
GSRELMMFYI DLKQTNDVLL TFEALKHLAS LLLHNKFATE FVAHGGVQKL
              410 420 430 440 450
LEIPRPSMAA TGVSMCLYYL SYNQDAMERV CMHPHNVLSD VVNYTLWLME
              460 470 480 490 500
CSHASGCCHA TMFFSICFSF RAVLELFDRY DGLRRLVNLI STLEILNLED
              510 520 530 540 550
QGALLSDDEI FASRQTGKHT CMALRKYFEA HLAIKLEQVK QSLQRTEGGI
              560 570 580 590 600
LVHPQPPYKA CSYTHEQIVE MMEFLIEYGP AQLYWEPAEV FLKLSCVQLL
              610 620 630 640 650
LQLISIACNW KTYYARNDTV RFALDVLAIL TVVPKIQLQL AESVDVLDEA
              660 670 680 690 700
GSTVSTVGIS IILGVAEGEF FIHDAEIQKS ALQIIINCVC GPDNRISSIG
              710 720 730 740 750
KFISGTPRRK LPQNPKSSEH TLAKMWNVVQ SNNGIKVLLS LLSIKMPITD
              760 770 780 790 800
ADQIRALACK ALVGLSRSST VRQIISKLPL FSSCQIQQLM KEPVLQDKRS
              810 820 830 840 850
DHVKFCKYAA ELIERVSGKP LLIGTDVSLA RLQKADVVAQ SRISFPEKEL
              860 870 880 890 900
LLLIRNHLIS KGLGETATVL TKEADLPMTA ASHSSAFTPV TAAASPVSLP
              910 920 930 940 950
RTPRIANGIA TRLGSHAAVG ASAPSAPTAH PQPRPPQGPL ALPGPSYAGN
              960 970 980 990 1000
SPLIGRISFI RERPSPCNGR KIRVLRQKSD HGAYSQSPAI KKQLDRHLPS
            1010 1020 1030 1040 1050
PPTLDSIITE YLREQHARCK NPVATCPPFS LFTPHQCPEP KQRRQAPINF
            1060 1070 1080 1090 1100
TSRLNRRASF PKYGGVDGGC FDRHLIFSRF RPISVFREAN EDESGFTCCA
            1110 1120 1130 1140 1150
FSARERFLML GTCTGQLKLY NVFSGQEEAS YNCHNSAITH LEPSRDGSLL
            1160 1170 1180 1190 1200
LTSATWSQPL SALWGMKSVF DMKHSFTEDH YVEFSKHSQD RVIGTKGDIA
            1210 1220 1230 1240 1250
HIYDIQTGNK LLTLFNPDLA NNYKRNCATF NPTDDLVLND GVLWDVRSAQ
            1260 1270 1280 1290 1300
AIHKFDKFNM NISGVFHPNG LEVIINTEIW DLRTFHLLHT VPALDQCRVV
            1310 1320 1330 1340 1350
FNHTGTVMYG AMLQADDEDD LMEERMKSPF GSSFRTFNAT DYKPIATIDV
            1360 1370 1380 1390 1400
KRNIFDLCTD TKDCYLAVIE NQGSMDALNM DTVCRLYEVG RQRLAEDEDE
            1410 1420 1430 1440 1450
EEDQEEEEQE EEDDDEDDDD TDDLDELDTD QLLEAELEED DNNENAGEDG
            1460 1470 1480 1490 1500
DNDFSPSDEE LANLLEEGED GEDEDSDADE EVELILGDTD SSDNSDLEDD
IILSLNE
```

### Isoform 2: (Identifier: Q9Y4B6-2)

The sequence of this isoform differs from the canonical sequence as follows:
87-87: Missing.

```
                10 20 30 40 50
MTTVVVHVDS KAELTTLLEQ WEKEHGSGQD MVPILTRMSQ LIEKETEEYR
                60 70 80 90 100
KGDPDPFDDR HPGRADPECM LGHLLRILFK NDDEMNLVNA YVMTSREPPL
              110 120 130 140 150
NTAACRLLLD IMPGLETAVV FQEKEGIVEN LFKWAREADQ PLRTYSTGLL
              160 170 180 190 200
GGAMENQDIA ANYRDENSQL VAIVLRRLRE LQLQEVALRQ ENKRPSPRKL
              210 220 230 240 250
SSEPLLPLDE EAVDMDYGDM AVDVVDGDQE EASGDMEISF HLDSGHKTSS
              260 270 280 290 300
RVNSTTKPED GGLKKNKSAK QGDRENFRKA KQKLGFSSSD PDRMFVELSN
              310 320 330 340 350
SSWSEMSPWV IGTNYTLYPM TPAIEQRLIL QYLTPLGEYQ ELLPIFMQLG
              360 370 380 390 400
SRELMMFYID LKQTNDVLLT FEALKHLASL LLHNKFATEF VAHGGVQKLL
              410 420 430 440 450
EIPRPSMAAT GVSMCLYYLS YNQDAMERVC MHPHNVLSDV VNYTLWLMEC
              460 470 480 490 500
SHASGCCHAT MFFSICFSFR AVLELFDRYD GLRRLVNLIS TLEILNLEDQ
              510 520 530 540 550
GALLSDDEIF ASRQTGKHTC MALRKYFEAH LAIKLEQVKQ SLQRTEGGIL
              560 570 580 590 600
VHPQPPYKAC SYTHEQIVEM MEFLIEYGPA QLYWEPAEVF LKLSCVQLLL
              610 620 630 640 650
QLISIACNWK TYYARNDTVR FALDVLAILT VVPKIQLQLA ESVDVLDEAG
              660 670 680 690 700
STVSTVGISI ILGVAEGEFF IHDAEIQKSA LQIIINCVCG PDNRISSIGK
              710 720 730 740 750
FISGTPRRKL PQNPKSSEHT LAKMWNVVQS NNGIKVLLSL LSIKMPITDA
              760 770 780 790 800
DQIRALACKA LVGLSRSSTV RQIISKLPLF SSCQIQQLMK EPVLQDKRSD
              810 820 830 840 850
HVKFCKYAAE LIERVSGKPL LIGTDVSLAR LQKADVVAQS RISFPEKELL
              860 870 880 890 900
LLIRNHLISK GLGETATVLT KEADLPMTAA SHSSAFTPVT AAASPVSLPR
              910 920 930 940 950
TPRIANGIAT RLGSHAAVGA SAPSAPTAHP QPRPPQGPLA LPGPSYAGNS
              960 970 980 990 1000
PLIGRISFIR ERPSPCNGRK IRVLRQKSDH GAYSQSPAIK KQLDRHLPSP
            1010 1020 1030 1040 1050
PTLDSIITEY LREQHARCKN PVATCPPFSL FTPHQCPEPK QRRQAPINFT
            1060 1070 1080 1090 1100
SRLNRRASFP KYGGVDGGCF DRHLIFSRFR PISVFREANE DESGFTCCAF
            1110 1120 1130 1140 1150
SARERFLMLG TCTGQLKLYN VESGQEEASY NCHNSAITHL EPSRDGSLLL
            1160 1170 1180 1190 1200
TSATWSQPLS ALWGMKSVED MKHSFTEDHY VEFSKHSQDR VIGTKGDIAH
            1210 1220 1230 1240 1250
IYDIQTGNKL LTLFNPDLAN NYKRNCATFN PTDDLVLNDG VLWDVRSAQA
            1260 1270 1280 1290 1300
IHKFDKENMN ISGVFHPNGL EVIINTEIWD LRTFHLLHTV PALDQCRVVE
            1310 1320 1330 1340 1350
NHTGTVMYGA MLQADDEDDL MEERMKSPFG SSFRTFNATD YKPIATIDVK
            1360 1370 1380 1390 1400
RNIFDLCTDT KDCYLAVIEN QGSMDALNMD TVCRLYEVGR QRLAEDEDEE
            1410 1420 1430 1440 1450
EDQEEEEQEE EDDDEDDDDT DDLDELDTDQ LLEAELEEDD NNENAGEDGD
            1460 1470 1480 1490 1500
NDFSPSDEEL ANLLEEGEDG EDEDSDADEE VELILGDTDS SDNSDLEDDI
ILSLNE
```

### Isoform 3 (Identifier: Q9Y4B6-3)

The sequence of this isoform differs from the canonical sequence as follows:
225-673: Missing.

```
                10 20 30 40 50
MTTVVVHVDS KAELTTLLEQ WEKEHGSGQD MVPILTRMSQ LIEKETEEYR
                60 70 80 90 100
KGDPDPFDDR HPGRADPECM LGHLLRILFK NDDEMNALVN AYVMTSREPP
              110 120 130 140 150
LNTAACRLLL DIMPGLETAV VFQEKEGIVE NLFKWAREAD QPLRTYSTGL
              160 170 180 190 200
LGGAMENQDI AANYRDENSQ LVAIVIRRLR ELQLQEVALR QENKRPSPRK
              210 220 230 240 250
LSSEPLLPLD EEAVDMDYGD MAVDDAEIQK SALQIIINCV CGPDNRISSI
              260 270 280 290 300
GKFISGTPRR KLPQNPKSSE HTLAKMWNVV QSNNGIKVLL SLLSIKMPIT
              310 320 330 340 350
DADQIRALAC KALVGLSRSS TVRQIISKLP LFSSCQIQQL MKEPVLQDKR
              360 370 380 390 400
SDHVKFCKYA AELIERVSGK PLLIGTDVSL ARLQKADVVA QSRISFPEKE
              410 420 430 440 450
LLLLIRNHLI SKGLGETATV LTKEADLPMT AASHSSAFTP VTAAASPVSL
              460 470 480 490 500
PRTPRIANGI ATRLGSHAAV GASAPSAPTA HPQPRPPQGP LALPGPSYAG
              510 520 530 540 550
NSPLIGRISF IRERPSPCNG RKIRVLRQKS DHGAYSQSPA IKKQLDRHLP
              560 570 580 590 600
SPPTLDSIIT EYLREQHARC KNPVATCPPF SLFTPHQCPE PKQRRQAPIN
              610 620 630 640 650
FTSRLNRRAS FPKYGGVDGG CFDRHLIFSR FRPISVFREA NEDESGFTCC
              660 670 680 690 700
AFSARERFLM LGTCTGQLKL YNVFSGQEEA SYNCHNSAIT HLEPSRDGSL
              710 720 730 740 750
LLTSATWSQP LSALWGMKSV FDMKHSFTED HYVEFSKHSQ DRVIGTKGDI
              760 770 780 790 800
AHIYDIQTGN KLLTLENPDL ANNYKRNCAT FNPTDDLVLN DGVLWDVRSA
              810 820 830 840 850
QAIHKFDKFN MNISGVFHPN GLEVIINTEI WDLRTFHLLH TVPALDQCRV
              860 870 880 890 900
VFNHTGTVMY GAMLQADDED DLMEERMKSP FGSSFRTENA TDYKPIATID
              910 920 930 940 950
VKRNIFDLCT DTKDCYLAVI ENQGSMDALN MDTVCRLYEV GRQRLAEDED
              960 970 980 990 1000
EEEDQEEEEQ EEEDDDEDDD DIDDLDELDT DQLLEAELEE DDNNENAGED
            1010 1020 1030 1040 1050
GDNDFSPSDE ELANLLEEGE DGEDEDSDAD EEVELILGDT DSSDNSDLED
DIILSLNE
```

### General Examples for the Processes and Compounds of the Invention

So far, efforts to expand the scope of E3 ligases addressable with small-molecule ligands have largely relied on phenotypic screening¹⁵⁻¹⁷ or focused studies of purified E3 ligases¹⁸⁻¹⁹. This work has uncovered the potential to target a range of E3 ligases with covalent chemistry²⁰, including DCAF16, DCAF11, RNF4, FEM1B, and RNF114¹⁵⁻¹⁹, which has enabled the design of electrophilic PROTACs that can promote targeted protein degradation at remarkably low stoichiometric engagement of the E3 ligase¹⁵⁻¹⁶ (reflecting the high catalytic potential of electrophilic PROTACs⁹). Nonetheless, the E3 ligase-targeting components of most of these PROTACs represent simple fragments bearing highly reactive cysteine-directed electrophiles such as a-chloroacetamides, and it therefore remains unclear whether such small molecule-E3 ligase interactions can be progressed to more advanced, selective chemical probes. Indeed, in multiple cases (e.g., DCAF11 and DCAF16), initial data suggest that the electrophilic PROTACs may have the capacity to engage more than one cysteine on the E3 ligase itself, underscoring the persistent challenges facing the discovery of selective electrophilic ligands for E3 ligases.

We recently introduced an activity-based protein profiling (ABPP) strategy for the chemical proteomic discovery of small molecule-protein interactions in human cells that leverages sets of stereoisomeric electrophilic compounds, wherein the interaction are prioritized based on a combination of cellular potency, stereochemical selectivity, and site-specificity²¹. Using these criteria, we discovered a striking number of cysteines on structurally and functionally diverse proteins that were stereoselectively engaged in primary human T cells by a focused set of tryptoline acrylamides. Here, we screened a distinct set of stereoisomeric azetidine acrylamides in human T cells using cysteine-directed mass spectrometry (MS)-ABPP²¹ These experiments identified a stereoselectively engaged cysteine (C1113) in the substrate binding domain of the Cullin4-RING E3 ligase (CRL4) substrate receptor DCAF1 (or VPRBP). We show that azetidine acrylamide-derived PROTACs promote the degradation of a target protein FBKP12 in a stereoselective manner that depends on DCAF1 and is blocked by mutation of C1113. We further demonstrate that the azetidine acrylamide PROTACs form a ternary complex with FKBP12 and DCAF1 and promote FBKP12 degradation at low fractional engagement (˜20%) of DCAF1_C1113. These findings, taken together, designate CRL4^{DCAF1} as an E3 ligase capable of supporting targeted protein degradation mediated by electrophilic PROTACS that act in a stereo- and site-selective manner.

### Discovery of a Stereo- and Site-Selective Electrophilic Compound-Cysteine Interaction in DCAF1.

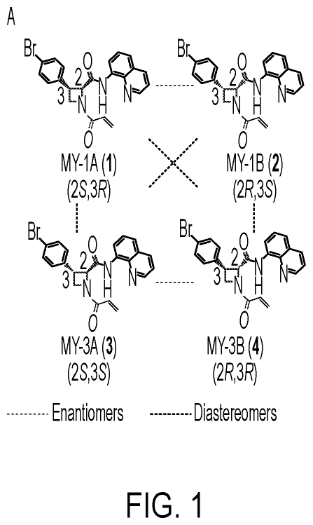

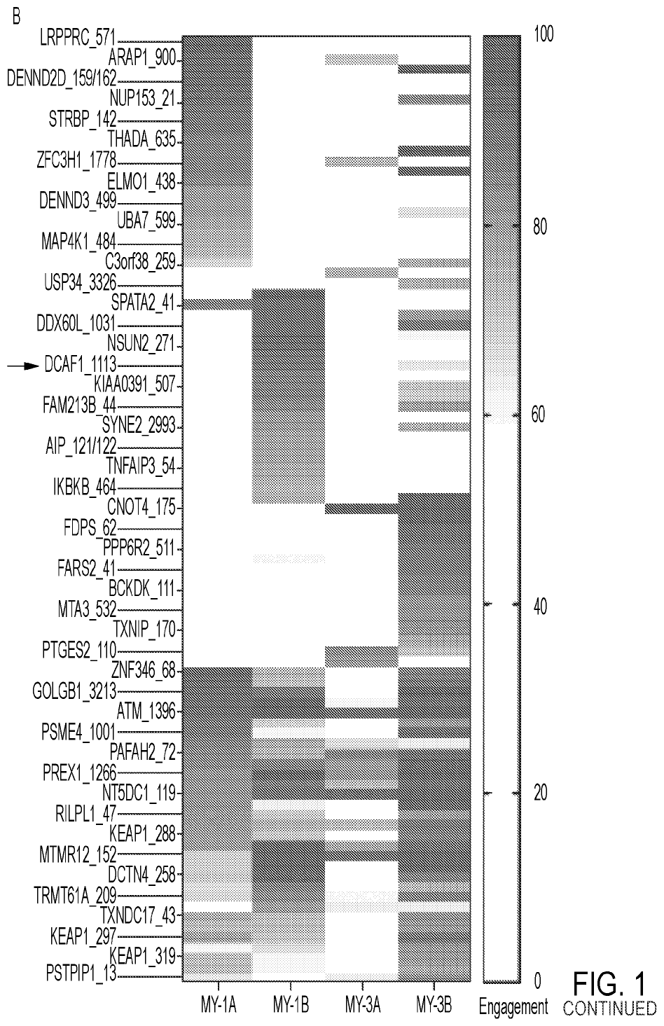

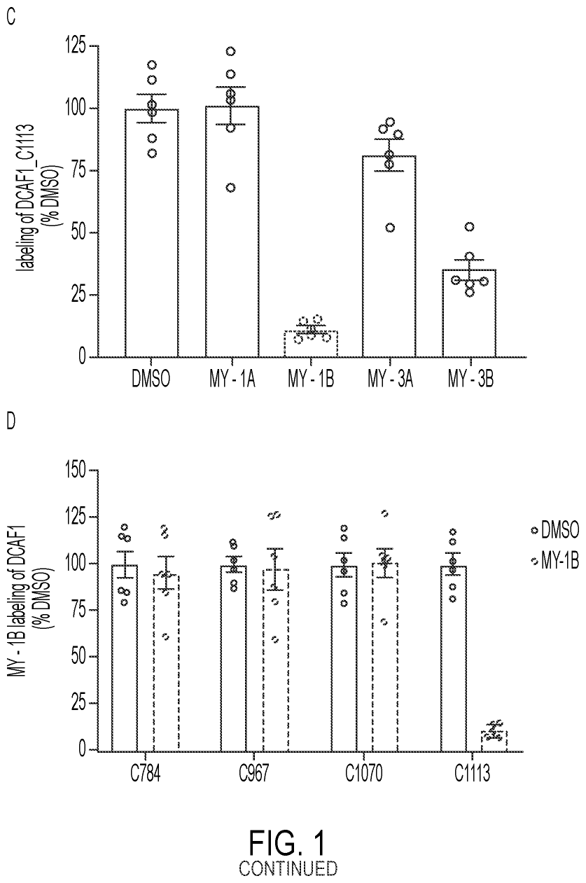

Guided by the principles of diversity-oriented synthesis²², specifically the design of compounds with densely functionalized and entropically constrained sp³-rich cores bearing one or more stereocenters, we generated a set of stereoisomeric azetidine acrylamides (FIG. 1A) and screened these compounds by cysteine-directed MS-ABPP²³⁻²⁶ in primary human T cells. Across more than 10000 quantified cysteines, these experiments uncovered several azetidine acrylamide-cysteine interactions (FIG. 1B and Dataset S1), including the stereo-(FIG. 1B, C) and site-(FIG. 1D) selective engagement of C1113 on the E3 ligase substrate adaptor DCAF1. Among the azetidine acrylamides, MY-1B (2) showed the strongest reactivity with DCAF1_C1113, while the enantiomer MY-1A (1) was inactive (FIG. 1B, C). A weaker enantioselective interaction was also observed for DCAF1_C1113 with MY-3B (4) compared to MY-3A (3) (FIG. 1B, C). Other quantified cysteines in DCAF1 were unaffected by MY-1B (FIG. 1D).

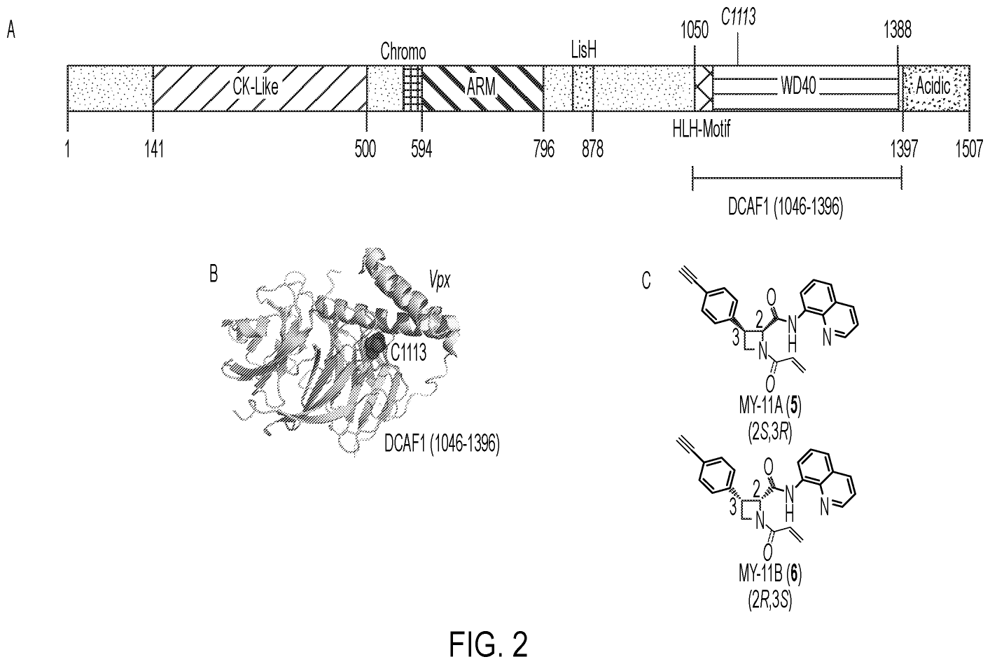

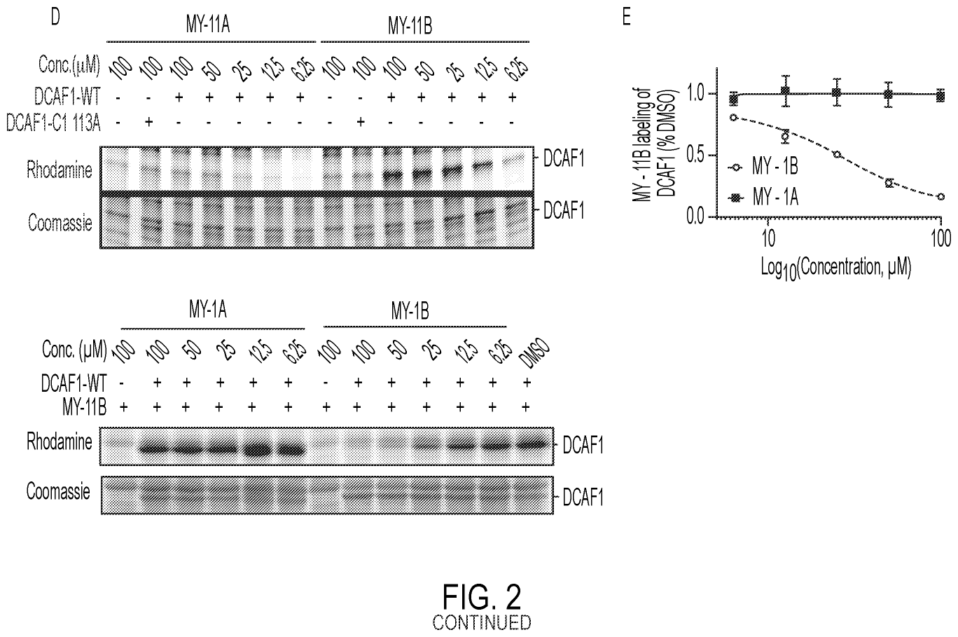

DCAF1 is a multi-domain substrate receptor for CRL4 ligases (FIG. 2A) that has diverse physiological and disease functions, including being co-opted by the HIV-2 virus to promote degradation of the antiviral host protein SAMHD1²⁷⁻²⁸. This outcome is achieved by interactions between DCAF1 and the viral protein Vpx, and, interestingly, C1113, which is located in the WD40 domain of DCAF1, resides in close proximity to the Vpx interface²⁹⁻³⁰ (FIG. 2B). We verified the stereo- and site-selective engagement of DCAF1_C1113 by azetidine acrylamides using alkynylated analogues of MY-1A and MY-1B (MY-11A (5) and MY-11B (6), respectively; FIG. 2C). For these experiments, we doped into HEK293T cell lysates a recombinantly expressed and purified form of DCAF1 containing the WD40 domain (amino acids (a.a.'s) 1046-1396), in complex with the CRL4 adaptor protein DDB1³¹, and exposed these samples to varying concentrations of MY-11A or MY-11B. After 1 h, we assessed MY-11A and MY-11B engagement of DCAF1 by copper-catalyzed azide-alkyne cycloaddition (CuAAC)³² with an azide-rhodamine reporter tag followed by SDS-PAGE and in-gel fluorescence scanning³³. This gel-ABPP experiment revealed much greater concentration-dependent labeling of recombinant DCAF1 by MY-11B versus MY-11A, and this labeling was blocked by mutation of C1113 to alanine (DCAF1-C1113A mutant) (FIG. 2D) or by pre-treatment with MY-1B, but not MY-1A (6.25-100 μM, 2 h pre-treatment; FIG. 2E, right). We used gel-ABPP to quantify an in vitro target engagement value (TE₅₀) of approximately 25 μM (95% C.I.=13-38 μM) for MY-1B labeling of DCAF1 (FIG. 2E, left). Modeling studies also supported a preferred interaction of DCAF1_C1113 with MY-1B over the other stereoisomers MY-1A, MY-3A, and MY-3B (Figure S1). These data, combined with our original MS-ABPP studies in T cells, indicate that MY-1B serves as a stereo- and site-selective covalent ligand for C1113 of DCAF1.

### Design and Characterization of DCAF1-Directed Electrophilic PROTACs.

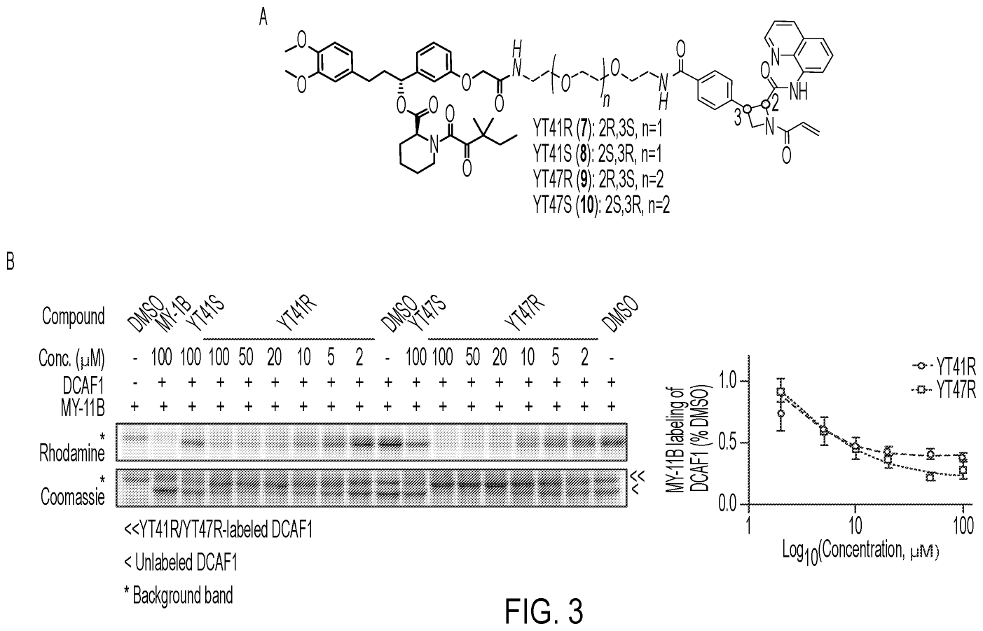

Considering the location of C1113 at the interface of DCAF1 binding to the viral Vpx protein, which in turn recruits the host antiviral protein SAMHD1 for ubiquitination and degradation³⁰, we hypothesized that covalent ligands targeting this cysteine might serve as the basis for PROTAC-mediated targeted protein degradation. The structural model for the MY-1B-DCAF1_C1113 complex suggested multiple potential exit vectors for PROTAC construction, including the para position of the phenyl ring and the acrylamide electrophile (Figure S2). For our initial studies, we elected to focus on constructing PROTACs that extended from the phenyl ring so that the acrylamide electrophile was left intact. We synthesized two heterobifunctional compounds—YT41R (7) and YT47R (9)—that connected MY-1B to the small molecule SLF (FIG. 3A), which is a high-affinity ligand for FKBP12 that is frequently used for assessing PROTAC performance^{15-16, 34-37}. YT41R and YT47R differed in the length of the PEG (polyethylene glycol) linker connecting the MY-1B and SLF units, and we also prepared two analogous control probes based on the inactive enantiomer MY-1A (YT41S (8) and YT47S (10); FIG. 3A).

Gel-ABPP experiments revealed that both YT41R and YT47R produced a concentration-dependent inhibition of MY-11B probe reactivity with recombinant DCAF1 (FIG. 3B). The blockade of MY-11B labeling plateaued at ˜75% at 20 M test concentrations of YT41R and YT47R, which could reflect the limited solubility of these candidate PROTACs at higher concentrations. In contrast, YT41S and YT47S did not impair the MY-11B-DCAF1 interaction (FIG. 3B). Interestingly, we also observed an apparent gel-shift for recombinant DCAF1 by Coomassie blue staining in the presence of YT41R or YT47R (but not YT41S or YT47S), likely reflecting the substantial molecular weight change in the protein caused by covalent reaction with these compounds, which have MWs>1000 Da (FIG. 3B, lower image).

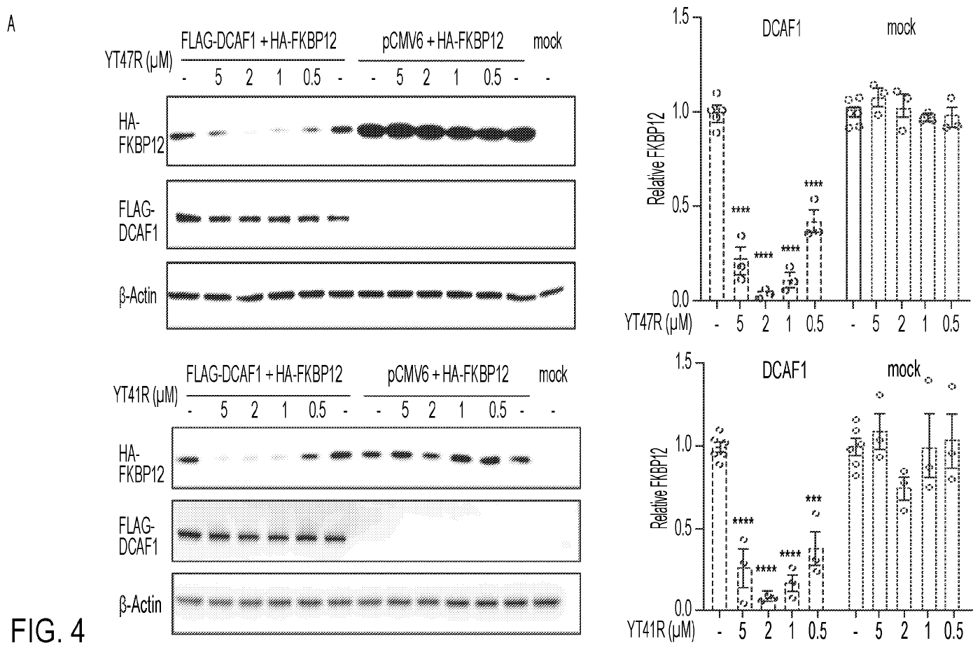

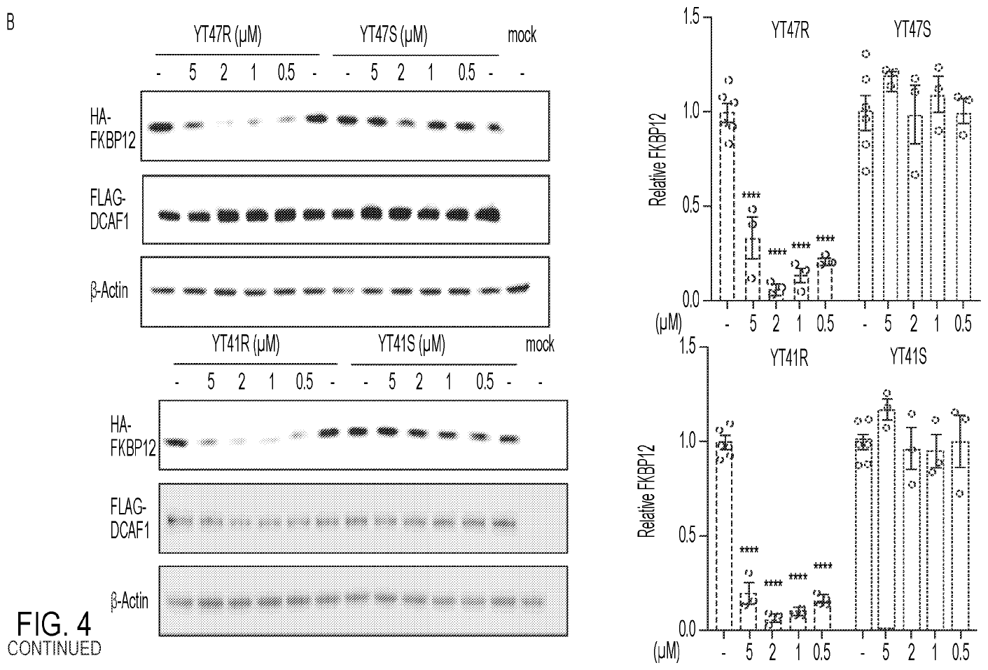

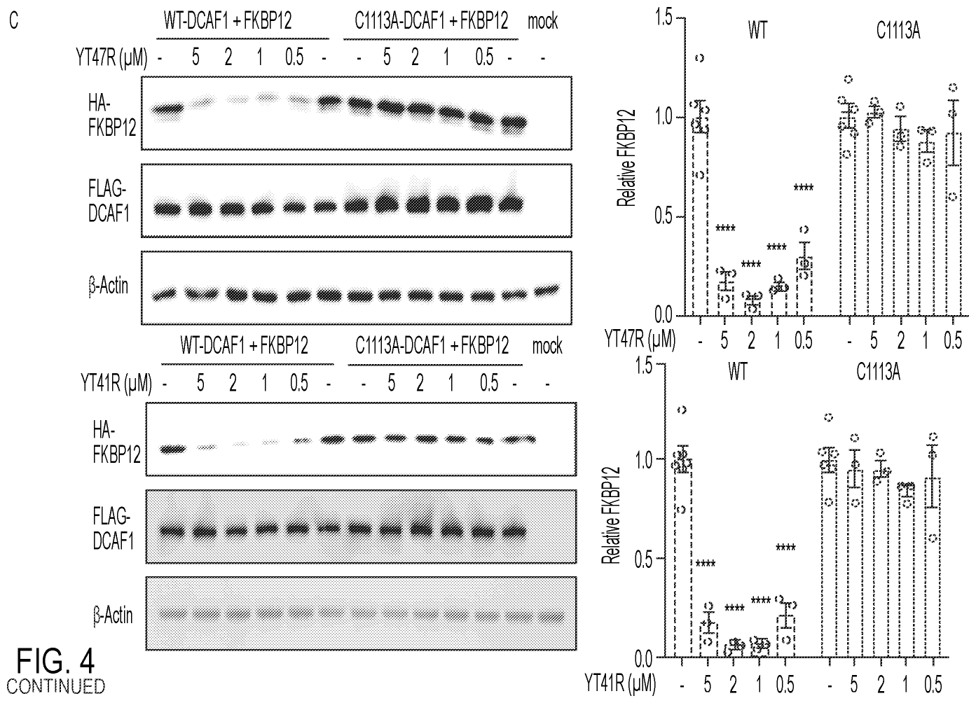

We next treated HEK293T cells co-transfected with FLAG epitope-tagged full length DCAF1 (a.a.'s 1-1507) and HA (hemagglutinin) epitope-tagged FKBP12 with a concentration range of YT41R or YT47R and, after 24 h, measured HA-FKBP12 abundance by Western blotting. Robust concentration-dependent degradation of HA-FKBP12 was observed for both YT41R and YT47R in co-transfected cells, but not HEK293T cells transfected only with HA-FKBP12 (FIG. 4A). Evidence of YT41R/YT47R-induced degradation of FKBP12 was observed at concentrations of YT41R and YT47R as low at 0.25 μM (Figure S3) with a possible modest hook effect emerging at 5 μM (FIG. 4A). Time-course studies revealed limited evidence of HA-FKBP12 degradation until ˜24 h after treatment of FLAG-DCAF1-transfected cells with YT41R and YT47R (Figure S4), which could reflect the time required for PROTAC-mediated degradation to overcome the robust expression of HA-FKBP12 in a transient co-transfection system.

YT41R and YT47R acted in a stereoselective manner, as neither of the control enantiomeric probes YT41S and YT47S supported HA-FKBP12 degradation (FIG. 4B). Additionally, we found that YT41R/YT47R did not promote the degradation of FKBP12 in cells expressing a DCAF1-C1113A mutant (FIG. 4C).

Taken together, these data indicate YT41R and YT47R act as electrophilic PROTACs that promote targeted protein degradation through the stereoselective engagement of C1113 of DCAF1.

### Mechanistic Studies of DCAF1-Directed Electrophilic PROTACs.

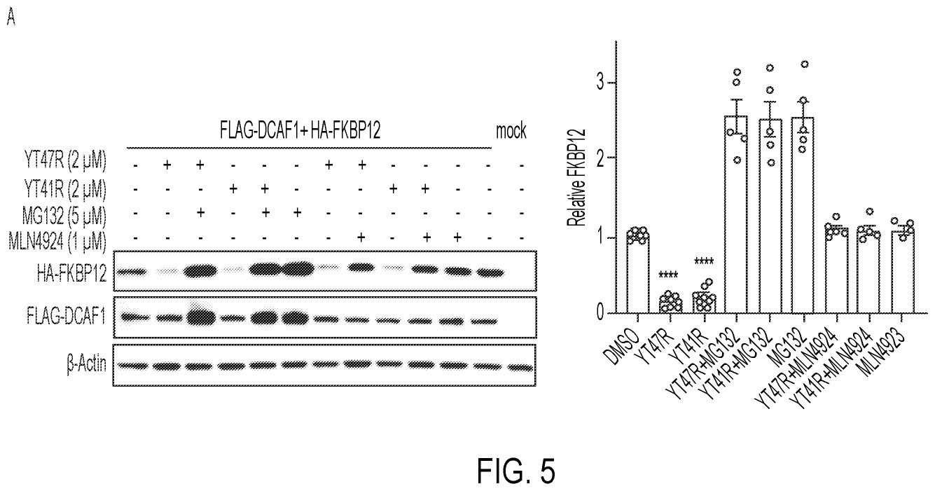

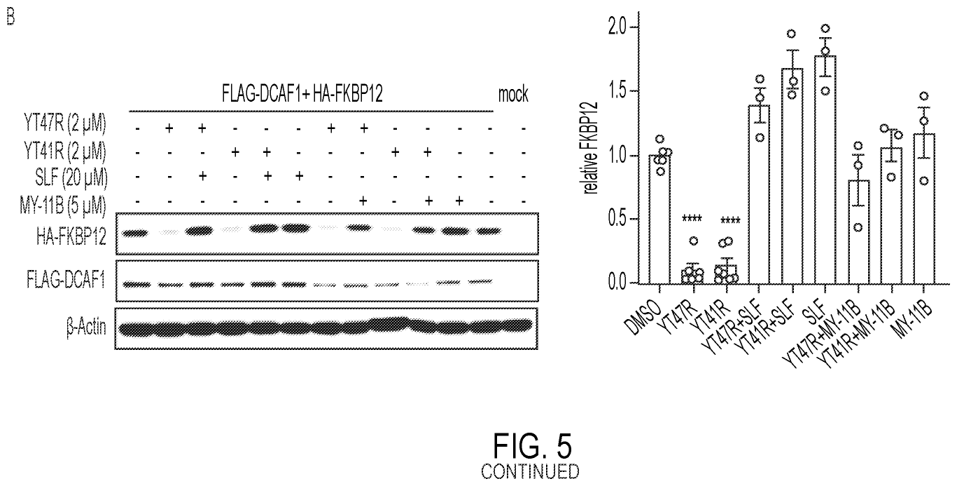

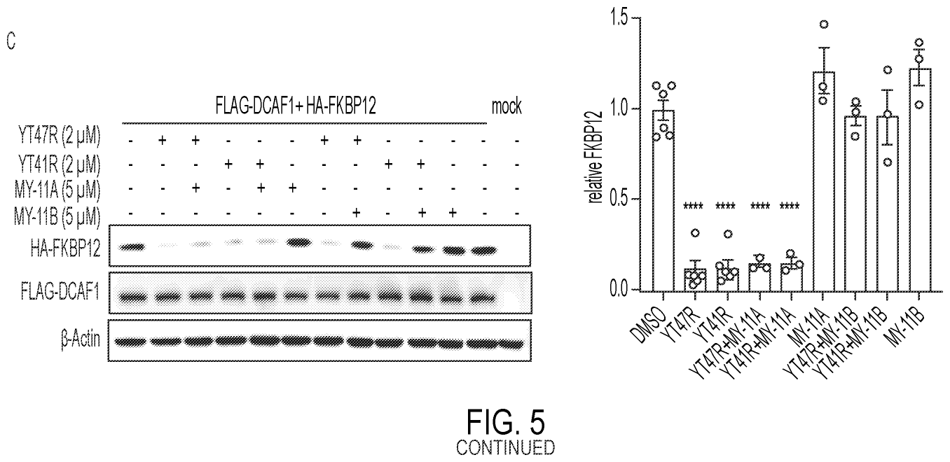

YT41R/YT47R-mediated degradation of HA-FKBP12 was blocked by pre-treatment with the proteasome inhibitor MG132 (5 μM, 1 h pre-treatment followed by 24 h co-treatment with YT41R/YT47R) (FIG. 5A); however, interpretation of these experiments was complicated by the increased quantities of both HA-FKBP12 and FLAG-DCAF1 observed in MG132-treated cells (FIG. 5A). On the other hand, treatment with the NAE inhibitor MLN4924, which blocks the Neddylation and activity of cullin-RING E3 ligases³⁸, also impaired YT41R/YT47R-mediated degradation of HA-FKBP12 without causing alterations in the quantity of HA-FKBP12 or FLAG-DCAF1 in control cells (FIG. 5A). These data support that the targeted protein degradation activity of YT41R and YT47R is proteasome- and cullin-RING E3 ligase-dependent.

PROTAC-mediated protein degradation requires ternary complex formation between the target protein, the heterobifunctional compound, and the E3 ligase, and this process can often be competitively disrupted by monovalent ligands targeting individual components of the complex. Consistent with this model for YT41R/YT47R action, we found that pre-treatment of HA-FKBP12 and FLAG-DCAF1 co-transfected HEK293T cells with either the FKBP12 ligand SLF (20 μM) or the DCAF1 ligand MY-11B (5 μM) blocked YT41R and YT47R-mediated degradation of HA-FKBP12 (FIG. 5B). The control enantiomer MY-11A, which does not engage DCAF1 (FIG. 2D), did not block YT41R/YT47R-mediated HA-FKBP12 degradation (FIG. 5C).

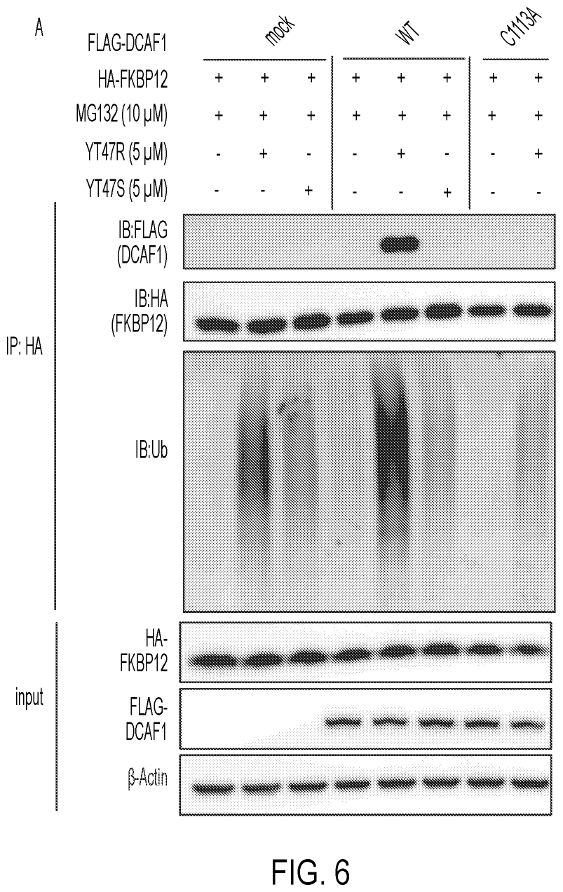

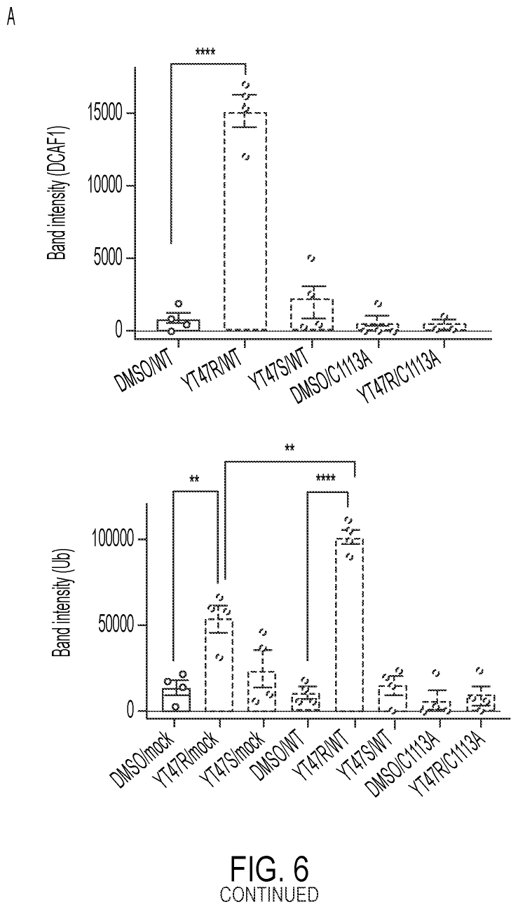

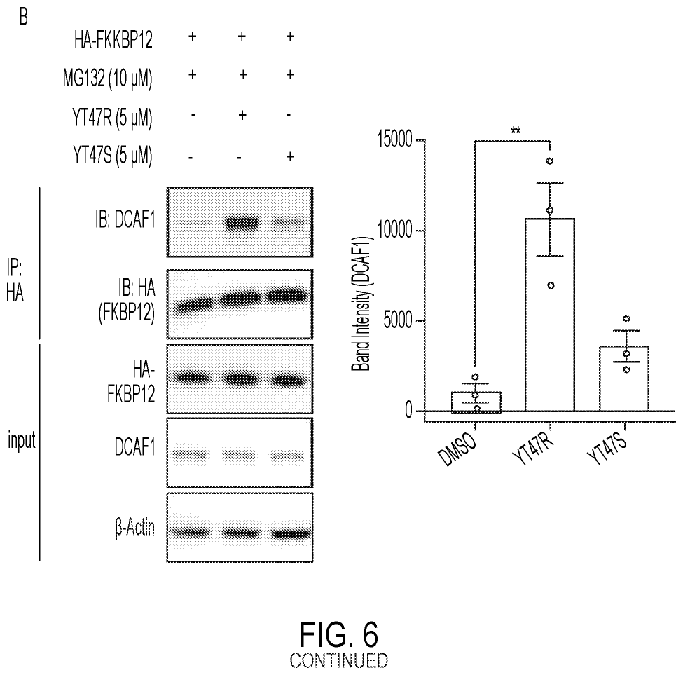

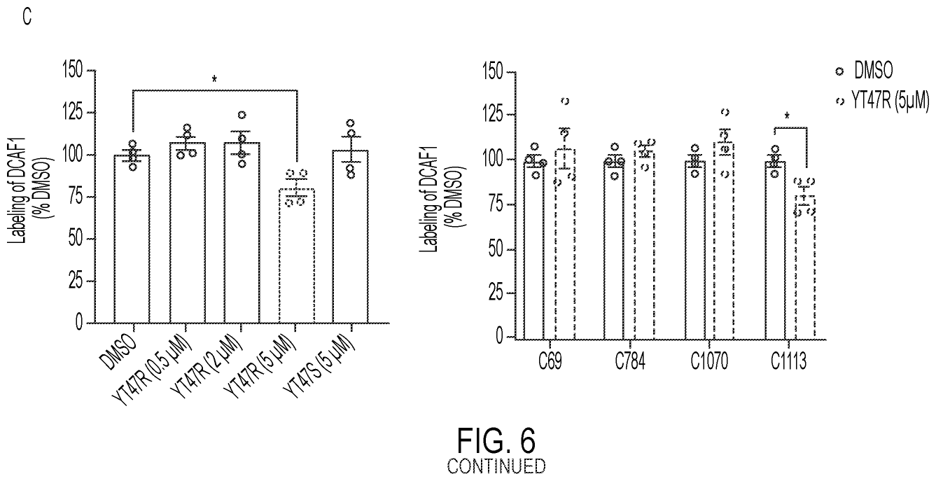

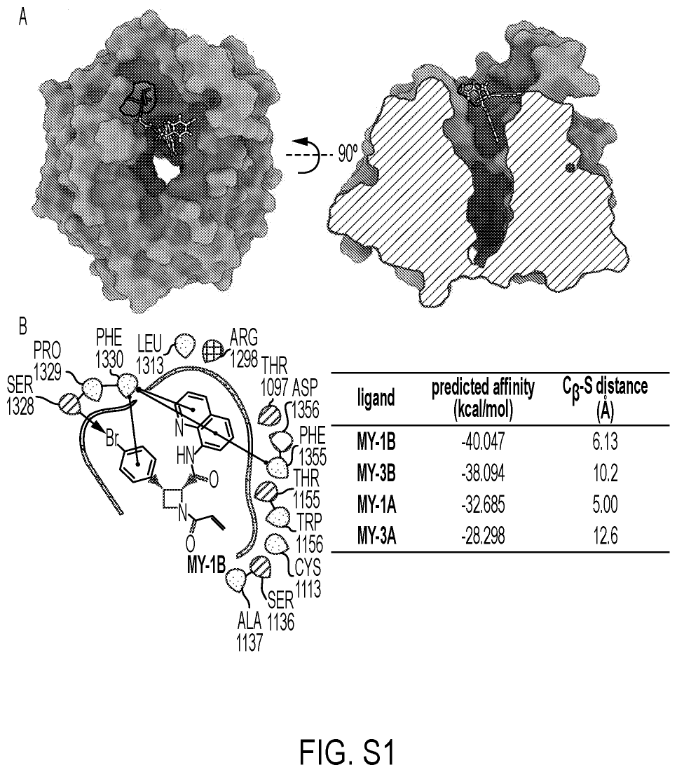

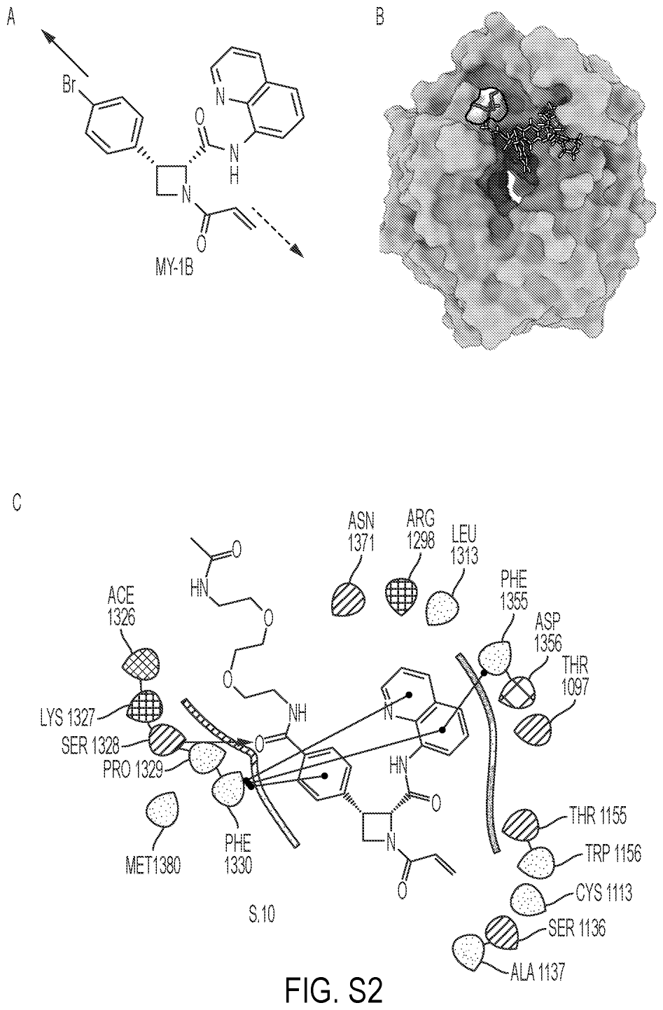

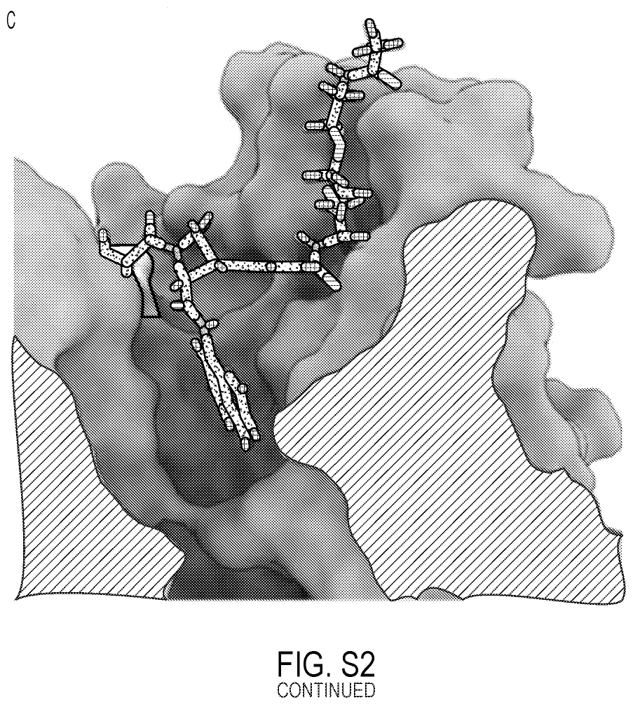

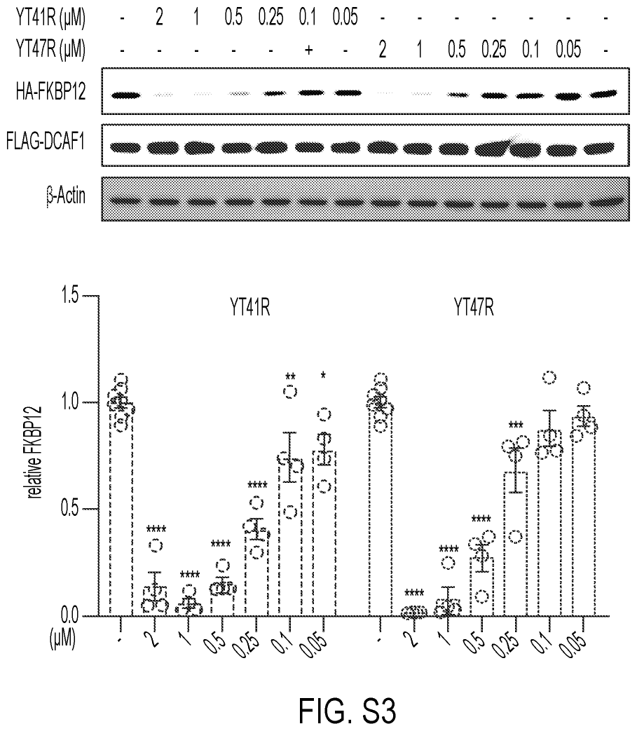

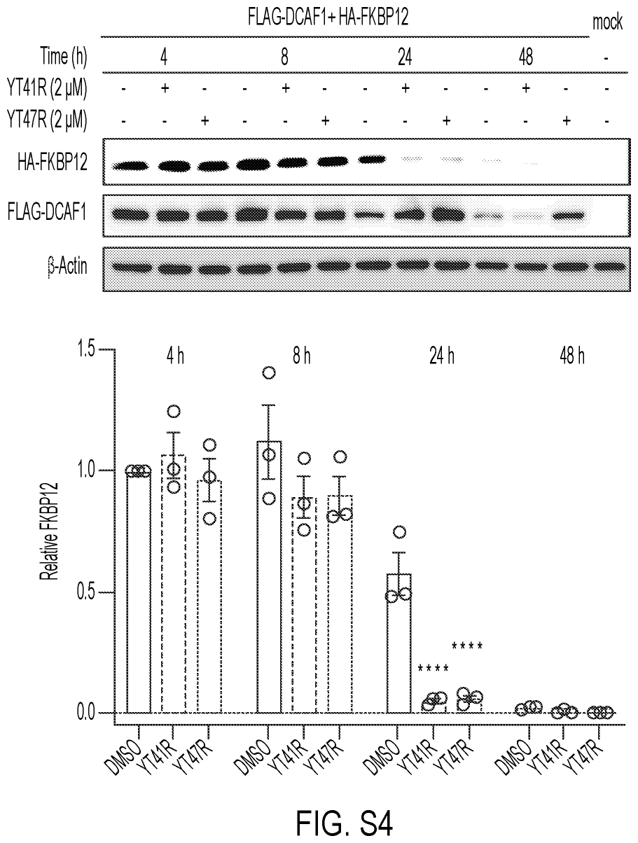

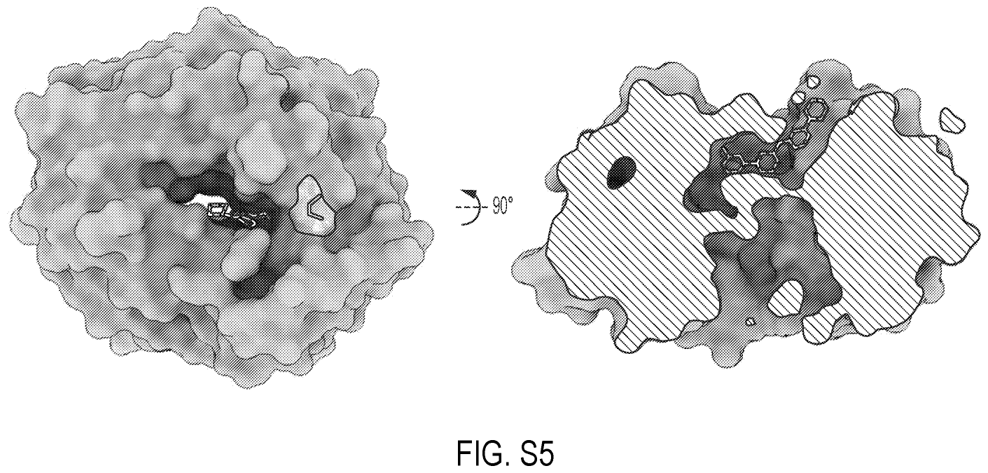

Finally, we investigated whether electrophilic PROTACs induce a stable ternary complex of FKBP12 with DCAF1 and promote FKBP12 ubiquitination. These co-immunoprecipitation experiments were performed in HEK293T cells co-transfected with HA-FKBP12 and FLAG-DCAF1 and treated with YT47R (or the control enantiomer YT47S) in the presence of MG132 (to block FKBP12 degradation) and revealed clear evidence of a ternary complex of FKBP12 and DCAF1 with YT47R, but not YT47S (FIG. 6A). A ternary complex was not observed in cells expressing the DCAF1-C1113A mutant (FIG. 6A), supporting that YT47R induces this complex through site-specifically reacting with C1113. We also detected robust polyubiquitination of the immunoprecipitated HA-FKBP12 in YT47R-treated, DCAF1-WT-expressing cells compared to YT47S- or DMSO-treated, DCAF1-WT-expressing cells or YT47R-treated, DCAF1-C1113A-expressing cells (FIG. 6A). Interestingly, we also observed increased FKBP12 ubiquitination in YT47R-versus YT47S-treated HEK293T cells that did not express exogenous DCAF1 (FIG. 6A). We wondered whether this outcome might be mediated by endogenous DCAF1, and, consistent with this possibility, we found that endogenous DCAF1 formed a more robust ternary complex with HA-FKBP12 in HEK293T cells treated with YT47R compared to YT47S or DMSO (FIG. 6B). In considering possible reasons why this ternary complex formation with endogenous DCAF1 was insufficient to support the degradation of recombinantly expressed FKBP12 (FIG. 4A), we speculate that our current PROTACs (YT41R, YT47R) may require further improvements in cellular properties to enable robust targeted protein degradation mediated by physiological concentrations of DCAF1. Possibly supporting this hypothesis, MS-ABPP experiments revealed that YT47R (5 μM) only engaged ˜20% of C1113 of recombinantly expressed DCAF1 (FIG. 6C and Data set S2). This minor engagement, which is consistent with previous studies of other electrophilic PROTACs that also act sub-stoichiometrically (and presumably catalytically) to degrade proteins¹⁵⁻¹⁶, was both stereo- and site-selective (FIG. 6C) and suggests more optimized PROTACs that engage a greater fraction of DCAF1_C1113 in cells may enable targeted protein degradation by endogenous DCAF1.

Here, we have described the chemical proteomic discovery of a ligandable cysteine (C1113) in the E3 ligase substrate receptor protein DCAF1 and the conversion of azetidine acrylamides targeting this cysteine into electrophilic PROTACs. These findings are significant because, to our knowledge, they describe the first electrophilic PROTACs that have been shown to act in a stereo- and site-selective manner. The properties of stereoselectivity and site-specificity provide convenient ways to establish controls to verify on-target activity for electrophilic PROTACs, as we have shown herein for DCAF1 and others have demonstrated for reversibly binding PROTACs that engage VHL in a stereoselective manner³⁹. We further interpret these features to indicate the presence of a high-quality druggable pocket in proximity to DCAF1_C1113. Also supportive of this conclusion is the recent report of a noncovalent DCAF1 ligand that binds a pocket near C1113 (PDB code: 7SSE, Figure S5). While we have taken advantage of the druggability of DCAF1_C1113 to create electrophilic PROTACs, we also imagine that more advanced covalent ligands might serve as molecular glues⁵⁻⁷ or antagonists of the various physiological and pathological functions of DCAF1⁴⁰⁻⁴⁴. Indeed, small-molecule antagonists of DCAF1 could counteract the pathogenesis of viruses like HIV-1 and HIV-2 that co-opt this E3 ligase substrate receptor to suppress immune cell responses^{30, 44-47}. More generally, the rich dataset of stereoselective interactions reported herein between azetidine acrylamides and cysteines in the human T-cell proteome (FIG. 1B and Dataset S1) should offer attractive starting points for chemical probe discovery for additional proteins from structurally and functionally diverse classes.

As has been described previously¹⁵⁻¹⁹, electrophilic PROTACs have the potential to maximally leverage the catalytic potential of targeted protein degradation by creating “neo”-E3 ligases that are permanently modified (until physical turnover) with a substrate-binding compound. However, key variables can impact the success of such endeavors, including the half-life and substrate compatibility of the E3 ligase¹¹⁻¹⁴, as well as the quality of the chemical probes that target it. On the latter point, we view the azetidine acrylamide reactive group found herein to stereoselectively engage DCAF1_C1113 as an encouraging starting point for probe optimization, especially in comparison to electrophilic PROTACs reported to target other E3 ligases, which mostly use high-reactivity groups such as a-chloroacetamides^{15-16, 18-19}. Nonetheless, we have, so far, only observed targeted protein degradation for electrophilic PROTACs in cells expressing recombinant DCAF1, and it will be important in future studies to determine if this activity can be extended to endogenous DCAF1. That we observed evidence of electrophilic PROTACs promoting a ternary complex with endogenous DCAF1 and FBKP12, as well as inducing FKBP12 ubiquitination, is encouraging, even though these effects were not apparently robust enough to lead to substantial FKBP12 degradation in cells expressing endogenous DCAF1 (possibly due to counteracting cellular deubiquitinases⁹). Further improvements in electrophilic PROTAC performance may require greater levels of cellular engagement of DCAF1, as our first-generation compounds only appear to modify≤20% of recombinant DCAF1 at functional concentrations in cells. A recent study has also described an autoinhibitory oligomerization mechanism for DCAF1 that may not be shared by other CRL4 substrate receptors⁴⁸. If a substantial proportion of endogenous DCAF1 is in an autoinhibited tetrameric state, then a greater quantity of total DCAF1 may need to be modified by electrophilic PROTACs to support targeted protein degradation. On the other hand, we wonder whether this autoregulatory feature of DCAF1 might also be exploited to create electrophilic PROTACs and/or molecular glues that carry out context-dependent protein degradation only in those cell types where DCAF1 is in an activated state.

### REFERENCES

### References

1 Bondeson, D. P.; Crews, C. M., Targeted Protein Degradation by Small Molecules. Annu. Rev. Pharmacol. Toxicol. 2017, 57, 107-123.
2. Schapira, M.; Calabrese, M. F.; Bullock, A. N.; Crews, C. M., Targeted protein degradation: expanding the toolbox. Nat Rev Drug Discov 2019, 18 (12), 949-963.
3. Kostic, M.; Jones, L. H., Critical Assessment of Targeted Protein Degradation as a Research Tool and Pharmacological Modality. Trends Pharmacol. Sci. 2020, 41 (5), 305-317.
4. Sun, X.; Gao, H.; Yang, Y.; He, M.; Wu, Y.; Song, Y.; Tong, Y.; Rao, Y., PROTACs: great opportunities for academia and industry. Signal Transduct Target Ther 2019, 4 (1), 64.
5. Schreiber, S. L., The Rise of Molecular Glues. Cell 2021, 184 (1), 3-9.
6 Kozicka, Z.; Thoma, N. H., Haven't got a glue: Protein surface variation for the design of molecular glue degraders. Cell Chem Biol 2021, 28 (7), 1032-1047.
7. Dong, G.; Ding, Y.; He, S.; Sheng, C., Molecular Glues for Targeted Protein Degradation: From Serendipity to Rational Discovery. J. Med. Chem. 2021, 64 (15), 10606-10620.
8. Bekes, M.; Langley, D. R.; Crews, C. M., PROTAC targeted protein degraders: the past is prologue. Nat. Rev. Drug Discovery 2022, 1-20.
9 Bondeson, D. P.; Mares, A.; Smith, I. E.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M., Catalytic in vivo protein knockdown by small-molecule PROTACs. Nat. Chem. Biol. 2015, 11 (8), 611-7.
10. Winter, G. E.; Buckley, D. L.; Paulk, J.; Roberts, J. M.; Souza, A.; Dhe-Paganon, S.; Bradner, J. E., DRUG DEVELOPMENT. Phthalimide conjugation as a strategy for in vivo target protein degradation. Science 2015, 348 (6241), 1376-81.
11. Lai, A. C.; Toure, M.; Hellerschmied, D.; Salami, J.; Jaime-Figueroa, S.; Ko, E.; Hines, J.; Crews, C. M., Modular PROTAC design for the degradation of oncogenic BCR-ABL. Angew. Chem., Int. Ed. 2016, 55 (2), 807-810.
12. Bondeson, D. P.; Smith, B. E.; Burslem, G. M.; Buhimschi, A. D.; Hines, J.; Jaime-Figueroa, S.; Wang, J.; Hamman, B. D.; Ishchenko, A.; Crews, C. M., Lessons in PROTAC Design from Selective Degradation with a Promiscuous Warhead. Cell Chem Biol 2018, 25 (1), 78-87 e5.
13. Huang, H.-T.; Dobrovolsky, D.; Paulk, J.; Yang, G.; Weisberg, E. L.; Doctor, Z. M.; Buckley, D. L.; Cho, J.-H.; Ko, E.; Jang, J., A chemoproteomic approach to query the degradable kinome using a multi-kinase degrader. Cell chemical biology 2018, 25 (1), 88-99. e6.
14. Donovan, K. A.; Ferguson, F. M.; Bushman, J. W.; Eleuteri, N. A.; Bhunia, D.; Ryu, S.; Tan, L.; Shi, K.; Yue, H.; Liu, X., Mapping the degradable kinome provides a resource for expedited degrader development. Cell 2020, 183 (6), 1714-1731. e10.
15. Zhang, X.; Luukkonen, L. M.; Eissler, C. L.; Crowley, V. M.; Yamashita, Y.; Schafroth, M. A.; Kikuchi, S.; Weinstein, D. S.; Symons, K. T.; Nordin, B. E.; Rodriguez, J. L.; Wucherpfennig, T. G.; Bauer, L. G.; Dix, M. M.; Stamos, D.; Kinsella, T. M.; Simon, G. M.; Baltgalvis, K. A.; Cravatt, B. F., DCAF11 Supports Targeted Protein Degradation by Electrophilic Proteolysis-Targeting Chimeras. J. Am. Chem. Soc. 2021, 143 (13), 5141-5149.
16. Zhang, X.; Crowley, V. M.; Wucherpfennig, T. G.; Dix, M. M.; Cravatt, B. F., Electrophilic PROTACs that degrade nuclear proteins by engaging DCAF16. Nat. Chem. Biol. 2019, 15 (7), 737-746.
17. Spradlin, J. N.; Hu, X.; Ward, C. C.; Brittain, S. M.; Jones, M. D.; Ou, L.; To, M.; Proudfoot, A.; Ornelas, E.; Woldegiorgis, M.; Olzmann, J. A.; Bussiere, D. E.; Thomas, J. R.; Tallarico, J. A.; McKenna, J. M.; Schirle, M.; Maimone, T. J.; Nomura, D. K., Harnessing the anti-cancer natural product nimbolide for targeted protein degradation. Nat. Chem. Biol. 2019, 15 (7), 747-755.
18. Ward, C. C.; Kleinman, J. I.; Brittain, S. M.; Lee, P. S.; Chung, C. Y. S.; Kim, K.; Petri, Y.; Thomas, J. R.; Tallarico, J. A.; McKenna, J. M.; Schirle, M.; Nomura, D. K., Covalent Ligand Screening Uncovers a RNF4 E3 Ligase Recruiter for Targeted Protein Degradation Applications. ACS Chem. Biol. 2019, 14 (11), 2430-2440.
19. Henning, N. J.; Manford, A. G.; Spradlin, J. N.; Brittain, S. M.; Zhang, E.; McKenna, J. M.; Tallarico, J. A.; Schirle, M.; Rape, M.; Nomura, D. K., Discovery of a Covalent FEM1B Recruiter for Targeted Protein Degradation Applications. J. Am. Chem. Soc. 2022, 144 (2), 701-708.
20. Gabizon, R.; London, N., The rise of covalent proteolysis targeting chimeras. Curr. Opin. Chem. Biol. 2021, 62, 24-33.
21. Vinogradova, E. V.; Zhang, X.; Remillard, D.; Lazar, D. C.; Suciu, R. M.; Wang, Y.; Bianco, G.; Yamashita, Y.; Crowley, V. M.; Schafroth, M. A.; Yokoyama, M.; Konrad, D. B.; Lum, K. M.; Simon, G. M.; Kemper, E. K.; Lazear, M. R.; Yin, S.; Blewett, M. M.; Dix, M. M.; Nguyen, N.; Shokhirev, M. N.; Chin, E. N.; Lairson, L. L.; Melillo, B.; Schreiber, S. L.; Forli, S.; Teijaro, J. R.; Cravatt, B. F., An Activity-Guided Map of Electrophile-Cysteine Interactions in Primary Human T Cells. Cell 2020, 182 (4), 1009-1026 e29.
22. Gerry, C. J.; Schreiber, S. L., Recent achievements and current trajectories of diversity-oriented synthesis. Curr. Opin. Chem. Biol. 2020, 56, 1-9.
23. Parker, C. G.; Galmozzi, A.; Wang, Y.; Correia, B. E.; Sasaki, K.; Joslyn, C. M.; Kim, A. S.; Cavallaro, C. L.; Lawrence, R. M.; Johnson, S. R.; Narvaiza, I.; Saez, E.; Cravatt, B. F., Ligand and Target Discovery by Fragment-Based Screening in Human Cells. Cell 2017, 168 (3), 527-541 e29.
24. Matthews, M. L.; He, L.; Horning, B. D.; Olson, E. J.; Correia, B. E.; Yates, J. R., 3^{rd}, Dawson, P. E.; Cravatt, B. F., Chemoproteomic profiling and discovery of protein electrophiles in human cells. Nat Chem 2017, 9 (3), 234-243.
25. Bar-Peled, L.; Kemper, E. K.; Suciu, R. M.; Vinogradova, E. V.; Backus, K. M.; Horning, B. D.; Paul, T. A.; Ichu, T. A.; Svensson, R. U.; Olucha, J.; Chang, M. W.; Kok, B. P.; Zhu, Z.; Ihle, N. T.; Dix, M. M.; Jiang, P.; Hayward, M. M.; Saez, E.; Shaw, R. J.; Cravatt, B. F., Chemical Proteomics Identifies Druggable Vulnerabilities in a Genetically Defined Cancer. Cell 2017, 171 (3), 696-709 e23.
26. Wang, Y.; Dix, M. M.; Bianco, G.; Remsberg, J. R.; Lee, H. Y.; Kalocsay, M.; Gygi, S. P.; Forli, S.; Vite, G.; Lawrence, R. M.; Parker, C. G.; Cravatt, B. F., Expedited mapping of the ligandable proteome using fully functionalized enantiomeric probe pairs. Nat Chem 2019, 11 (12), 1113-1123.
27. Schabla, N. M.; Mondal, K.; Swanson, P. C., DCAF1 (VprBP): emerging physiological roles for a unique dual-service E3 ubiquitin ligase substrate receptor. J Mol Cell Biol 2019, 11 (9), 725-735.
28. Chen, Z.; Zhang, L.; Ying, S., SAMHD1: a novel antiviral factor in intrinsic immunity. Future Microbiol 2012, 7 (9), 1117-26.
29. Schwefel, D.; Groom, H. C.; Boucherit, V. C.; Christodoulou, E.; Walker, P. A.; Stoye, J. P.; Bishop, K. N.; Taylor, I. A., Structural basis of lentiviral subversion of a cellular protein degradation pathway. Nature 2014, 505 (7482), 234-8.
30. Laguette, N.; Sobhian, B.; Casartelli, N.; Ringeard, M.; Chable-Bessia, C.; Segeral, E.; Yatim, A.; Emiliani, S.; Schwartz, O.; Benkirane, M., SAMHD1 is the dendritic- and myeloid-cell-specific HIV-1 restriction factor counteracted by Vpx. Nature 2011, 474 (7353), 654-U132.
31. Banchenko, S.; Krupp, F.; Gotthold, C.; Bürger, J.; Graziadei, A.; O'Reilly, F. J.; Sinn, L.; Ruda, O.; Rappsilber, J.; Spahn, C. M., Structural insights into Cullin4-RING ubiquitin ligase remodelling by Vpr from simian immunodeficiency viruses. PLOS Pathog. 2021, 17 (8), e1009775.
32. Rostovtsev, V. V.; Green, L. G.; Fokin, V. V.; Sharpless, K. B., A stepwise huisgen cycloaddition process: copper (I)-catalyzed regioselective “ligation” of azides and terminal alkynes. Angewandte Chemie 2002, 114 (14), 2708-2711.
33. Speers, A. E.; Adam, G. C.; Cravatt, B. F., Activity-based protein profiling in vivo using a copper (i)-catalyzed azide-alkyne [3+2] cycloaddition. J. Am. Chem. Soc. 2003, 125 (16), 4686-4687.
34. Nalawansha, D. A.; Li, K.; Hines, J.; Crews, C. M., Hijacking Methyl Reader Proteins for Nuclear-Specific Protein Degradation. BioRxiv https: doi.org 10.1101/2022.01.24.477424 2022.
35. Reynders, M.; Matsuura, B. S.; Bérouti, M.; Simoneschi, D.; Marzio, A.; Pagano, M.; Trauner, D., PHOTACs enable optical control of protein degradation. Science advances 2020, 6 (8), eaay5064.
36. Nabet, B.; Roberts, J. M.; Buckley, D. L.; Paulk, J.; Dastjerdi, S.; Yang, A.; Leggett, A. L.; Erb, M. A.; Lawlor, M. A.; Souza, A., The dTAG system for immediate and target-specific protein degradation. Nat. Chem. Biol. 2018, 14 (5), 431-441.
37. Winter, G. E.; Buckley, D. L.; Paulk, J.; Roberts, J. M.; Souza, A.; Dhe-Paganon, S.; Bradner, J. E., Selective target protein degradation via phthalimide conjugation. Science 2015, 348 (6241), 1376.
38. Soucy, T. A.; Smith, P. G.; Milhollen, M. A.; Berger, A. J.; Gavin, J. M.; Adhikari, S.; Brownell, J. E.; Burke, K. E.; Cardin, D. P.; Critchley, S.; Cullis, C. A.; Doucette, A.; Garnsey, J. J.; Gaulin, J. L.; Gershman, R. E.; Lublinsky, A. R.; McDonald, A.; Mizutani, H.; Narayanan, U.; Olhava, E. J.; Peluso, S.; Rezaei, M.; Sintchak, M. D.; Talreja, T.; Thomas, M. P.; Traore, T.; Vyskocil, S.; Weatherhead, G. S.; Yu, J.; Zhang, J.; Dick, L. R.; Claiborne, C. F.; Rolfe, M.; Bolen, J. B.; Langston, S. P., An inhibitor of NEDD8-activating enzyme as a new approach to treat cancer. Nature 2009, 458 (7239), 732-U67.
39. Bond, M. J.; Chu, L.; Nalawansha, D. A.; Li, K.; Crews, C. M., Targeted Degradation of Oncogenic KRAS (G12C) by VHL-Recruiting PROTACs. ACS Cent Sci 2020, 6 (8), 1367-1375.
40. Schabla, N. M.; Mondal, K.; Swanson, P. C., DCAF1 (VprBP): emerging physiological roles for a unique dual-service E3 ubiquitin ligase substrate receptor. Journal of molecular cell biology 2019, 11 (9), 725-735.
41. Nakagawa, T.; Mondal, K.; Swanson, P. C., VprBP (DCAF1): a promiscuous substrate recognition subunit that incorporates into both RING-family CRL4 and HECT-family EDD/UBR5 E3 ubiquitin ligases. Bmc Molecular Biology 2013, 14 (1), 1-12.
42. Li, W.; You, L.; Cooper, J.; Schiavon, G.; Pepe-Caprio, A.; Zhou, L.; Ishii, R.; Giovannini, M.; Hanemann, C. O.; Long, S. B., Merlin/NF2 suppresses tumorigenesis by inhibiting the E3 ubiquitin ligase CRL4DCAF1 in the nucleus. Cell 2010, 140 (4), 477-490.
43. McCall, C. M.; de Marval, P. L. M.; Chastain, P. D.; Jackson, S. C.; He, Y. J.; Kotake, Y.; Cook, J. G.; Xiong, Y., Human immunodeficiency virus type 1 Vpr-binding protein VprBP, a WD40 protein associated with the DDB1-CUL4 E3 ubiquitin ligase, is essential for DNA replication and embryonic development. Mol. Cell. Biol. 2008, 28 (18), 5621-5633.
44. Hrecka, K.; Gierszewska, M.; Srivastava, S.; Kozaczkiewicz, L.; Swanson, S. K.; Florens, L.; Washburn, M. P.; Skowronski, J., Lentiviral Vpr usurps Cul4-DDB1 [VprBP] E3 ubiquitin ligase to modulate cell cycle. Proceedings of the National Academy of Sciences 2007, 104 (28), 11778-11783.
45. Planelles, V.; Barker, E., Roles of Vpr and Vpx in modulating the virus-host cell relationship. Mol Aspects Med 2010, 31 (5), 398-406.
46. Greenwood, E. J.; Williamson, J. C.; Sienkiewicz, A.; Naamati, A.; Matheson, N. J.; Lehner, P. J., Promiscuous targeting of cellular proteins by Vpr drives systems-level proteomic remodeling in HIV-1 infection. Cell reports 2019, 27 (5), 1579-1596. e7.
47. Wu, Y.; Zhou, X.; Barnes, C. O.; DeLucia, M.; Cohen, A. E.; Gronenborn, A. M.; Ahn, J.; Calero, G., The DDB1-DCAF1-Vpr-UNG2 crystal structure reveals how HIV-1 Vpr steers human UNG2 toward destruction. Nat. Struct. Mol. Biol. 2016, 23 (10), 933-940.
48. Mohamed, W. I.; Schenk, A. D.; Kempf, G.; Cavadini, S.; Basters, A.; Potenza, A.; Abdul Rahman, W.; Rabl, J.; Reichermeier, K.; Thomä, N. H., The CRL4DCAF1 cullin-RING ubiquitin ligase is activated following a switch in oligomerization state. The EMBO journal 2021, 40 (22), e108008.

### EXAMPLES

### General Information

Compounds of the invention can be made by a variety of methods depicted in the illustrative synthetic reactions described below in this Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

All chemicals, including anhydrous solvents, were obtained from commercial suppliers and used without further purification. Merck silica gel TLC plates (0.25 mm, 60 F₂₅₄) were used to monitor reaction progress and were visualized under UV light (254 nm) or by staining with potassium permanganate (KmnO₄). Flash chromatography was performed using SiliaFlash® F60 silica gel (SiO₂, 40-60 μM, 60 Å), loaded with dichloromethane unless otherwise noted. NMR spectra were recorded on a Bruker 600 or 500 MHz instrument. All ¹H NMR chemical shifts (δ) are reported in parts per million (ppm) relative to residual signals for CHCl₃ (7.26 ppm) or CH₃OH (3.31 ppm) in the deuterated solvent. Reactions were monitored by LCMS and TLC. High-resolution mass spectra (HRMS) were obtained on an Agilent LC/MSD TOF mass spectrometer by electrospray ionization-time-of-flight (ESI-TOF). Analytical supercritical fluid chromatography (SFC) was performed on a Shimadzu LC system (flow rate: 3 mL/min, back pressure: 100 Bar, column temperature: 35° C.) equipped with a polydiode array detector unless otherwise noted. Tandem liquid chromatography/mass spectrometry (LC-MS) was performed on an Agilent 1200 series LC/MSD system equipped with an Agilent G6110A mass detector, alternatively a Shimadzu LC-20AD or AB series LC-MS system equipped with Shimadzu SPD-M20A or SPDM40 mass detectors, alternatively a Waters H-Class LC with equipped with diode array and Qda mass detector.

### Materials and Methods

All reactions were carried out under positive pressure of argon unless otherwise noted. Glassware was oven-dried at 120° C. for a minimum of 12 hours, or flame-dried with a propane torch under vacuum (<1 torr). Anhydrous dichloromethane (CH₂Cl₂) was distilled from calcium hydride (5% w/v) under positive pressure of nitrogen. Anhydrous tetrahydrofuran (THF) containing 250 ppm BHT (peroxide inhibitor) was purchased from MilliporeSigma/SigmaAldrich. Anhydrous toluene was obtained by passing the previously degassed solvent through an activated alumina column. Other commercially available solvents or reagents were used without further purification unless otherwise noted. Reactions were monitored by thin layer chromatography (TLC) using precoated silica gel plates from EMD Chemicals (TLC Silica gel 60 F₂₅₄, 250 μm thickness). Flash column chromatography was performed over Silica gel 60 (particle size 0.04-0.063 mm) from EMD Chemicals and activated neutral alumina (Brockmann I, 150 mesh) from Sigma-Aldrich. Room temperature or ambient temperature in Beckman Building, Lab 420 is 22° C. Organic solvent from crude reaction mixtures and solutions of pure compounds was evaporated on a Büchi Rotavapor R3 (rotavap or rotovap, referred to in the experimental procedures).

Hexanes (ACS grade), ethyl acetate (ACS grade), diethyl ether (anhydrous ACS grade), dichloromethane (ACS grade), chloroform (ACS grade), and isopropanol (ACS grade) were purchased from Fisher Chemical and used without further purification. Anhydrous tetrahydrofuran, DMF, and acetonitrile were purchased from Sigma-Aldrich. Anhydrous DMSO was purchased from Acros Organics. Anhydrous ethanol was obtained from Pharmco-Aaper. [Ir{dF(CF₃)ppy}₂(dtbbpy)]PF₆ was prepared according to the procedure of Lowry et al.¹ Photocatalysts 3CzCIIPN and 4CzIPN were prepared according to the procedure of Zeitler et al.² NiCl₂·glyme and IrCl₃·nH₂O were purchased from Strem Chemicals. Commercially available substrates were used without further purification unless otherwise noted. The reactions were monitored by thin layer chromatography (TLC) using precoated silica gel plates from EMD Chemicals (TLC Silica gel 60 F₂₅₄) or by LC/MS on an Agilent 6120 Quadrupole system with an ESI probe. Flash column chromatography was performed over Silica gel 60 (particle size 0.04-0.063 mm) from Fischer Scientific or Florsil® from Sigma Aldrich or Acros Organics. ¹H NMR and ¹³C NMR spectra were recorded on Bruker DPX-400, a Bruker DPX-500 or Bruker DPX-600 equipped with cryoprobe, and the residual solvent peaks were used as internal standard (CDCl₃: 7.26 ppm ¹H NMR, 77.16 ppm, DMSO-d₆: 2.50 ppm 1H NMR, 39.52 ppm, MeOH-d₄: 3.31 ppm ¹H NMR, 49.00 ppm 13C NMR. NMR data is denoted with apparent multiplicities: s=singlet, d=doublet, t=triplet, q=quartet, and combinations thereof.

### Abbreviations

DCAF1=DDB1-Cul4-associated factor 1; DDB1=DNA damage-binding protein 1; Cul4-Cullin-4A; FKBP12=FK506-binding protein 12; BRD4=Bromodomain-containing protein 4; IRAK4=interleukin-1 receptor-associated kinase 4; JAK=Janus kinase; BCL-XL=B-cell lymphoma-extra large; BCL-2=B-cell lymphoma 2; Stat3=Signal transducer and activator of transcription 3.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1, l′-bis-(diphenylphosphino) ethane (dppe), 1, l′-bis-(diphenylphosphino) ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂-(mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMezSi (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Synthesis of MY-1A

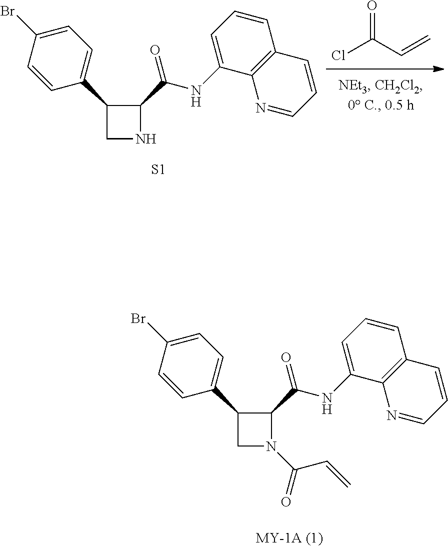

### (2S,3R)-1-acryloyl-3-(4-bromophenyl)-N-(84uinoline-8-yl) azetidine-2-carboxamide (MY-1A)

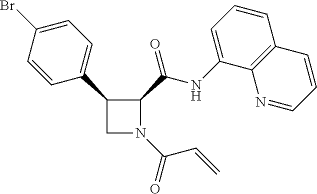 [SMILES: C=CC(=O)N1C[C@@H](c2ccc(Br)cc2)[C@H]1C(=O)Nc1cccc2cccnc12]

To a precooled (0° C.) solution of S1(50.0 mg, 131 μmol)⁴ in dichloromethane (1 mL) were added triethylamine (26.5 mg, 262 μmol) and acryloyl chloride (14.2 mg, 157 μmol). The mixture was stirred at 0° C. for 0.5 hours. Upon completion, the reaction mixture was concentrated under reduced pressure to obtain a residue, which was purified by prep-TLC (SiO₂, petroleum ether/EtOAc=2:1) to give MY-1A (51.0 mg, 89% yield) as a white solid. HRMS ESI-TOF m/z calculated for C₂₂H₁₉BrN₃O₂ [M+H]⁺ 436.0655. Found 436.0646. ¹H NMR (400 MHz, CDCl₃): δ 10.47 (br s, 1H), 8.81 (d, J=3.6 Hz, 1H), 8.43 (d, J=4.0 Hz, 1H), 8.16 (d, J=8.0 Hz, 1H), 7.54-7.42 (m, 3H), 7.29-7.25 (m, 4H+CHCl₃), 6.53 (d, J=16.3 Hz, 1H), 6.48-6.18 (m, 1H), 5.98-5.62 (m, 1H), 5.38 (d, J=8.0 Hz, 1H), 4.64 (t, J=9.3 Hz, 1H), 4.59-4.40 (m, 1H), 4.33-4.24 (m, 1H).

### Synthesis of MY-1B

### Prepared in Analogous Fashion from Ent-S1⁴

### (2R,3S)-1-acryloyl-3-(4-bromophenyl)-N-(84uinoline-8-yl) azetidine-2-carboxamide (MY-1B)

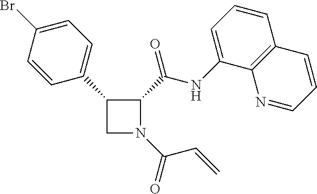 [SMILES: C=CC(=O)N1C[C@H](c2ccc(Br)cc2)[C@@H]1C(=O)Nc1cccc2cccnc12]

HRMS ESI-TOF m/z calculated for C₂₂H₁₉BrN₃O₂ [M+H]⁺ 436.0655. Found 436.0649. ¹H NMR (400 MHz, CDCl₃): δ 10.47 (s, 1H), 8.80 (d, J=4.0 Hz, 1H), 8.42 (d, J=4.0 Hz, 1H), 8.15 (d, J=8.0 Hz, 1H), 7.52-7.40 (m, 3H), 7.30-7.25 (m, 4H+CHCl₃), 6.64-6.15 (m, 2H), 5.90-5.64 (m, 1H), 5.37 (d, J=8.0 Hz, 1H), 4.63 (t, J=9.3 Hz, 1H), 4.57-4.38 (m, 1H), 4.33-4.22 (m, 1H).

### Synthesis of MY-3A

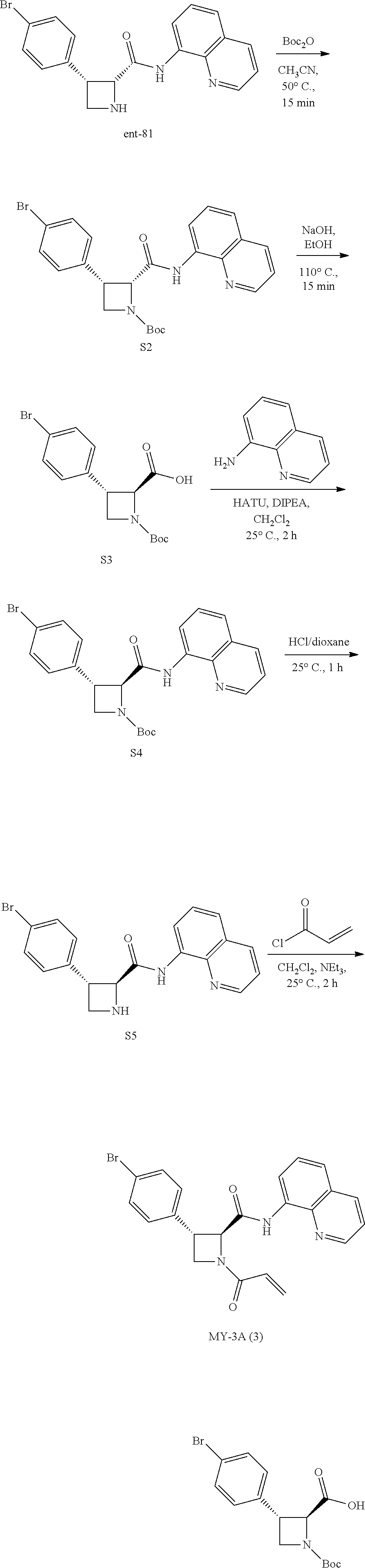

(2S,3S)-3-(4-bromophenyl)-1-(tert-butoxycarbonyl) azetidine-2-carboxylic acid (S3) To a solution of ent-S1 (450 mg, 1.18 mmol) in acetonitrile (3 mL) was added Boc₂O (308 mg, 1.41 mmol), and the resulting mixture was stirred at 50° C. for 15 min. Upon completion, the reaction mixture was filtered over Celite and concentrated under reduced pressure to give S2 (600 mg, crude) as a yellow oil. To a solution of S2 (450 mg, 933 μmol) in ethanol (3 mL) was added sodium hydroxide (373 mg, 9.33 mmol), and the resulting mixture was stirred at 110° C. for 15 min. Upon completion, the mixture was diluted with water and washed with dichloromethane (2×100 mL). The resulting aqueous solution was acidified with HCl (1 M) to adjust pH to 5˜6, exhaustively extracted with i-PrOH/CHCl₃ (3:7, 5×60 mL), dried over anhydrous sodium sulfate, filtered, and concentrated under reduced pressure to give S3 (330 mg, 99% yield over two steps) as a white solid.

LC-MS m/z calculated for C₁₅H₁₉BrNO₄ [M+H]⁺ 356.0. Found 356.0.

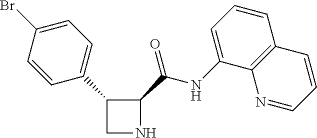 [SMILES: O=C(Nc1cccc2cccnc12)[C@H]1NC[C@@H]1c1ccc(Br)cc1]

(2S,3S)-3-(4-bromophenyl)-N-(86uinoline-8-yl) azetidine-2-carboxamide (S5) To a solution of S3 (330 mg, 926 μmol) in dichloromethane (4 mL) were added diisopropylethylamine (239 mg, 1.85 mmol) and 8-aminoquinoline (23.7 mg, 262 μmol), followed by HATU (705 mg, 1.85 mmol). The resulting mixture was stirred at 25° C. for 2 hours. Upon completion, the reaction mixture was concentrated under reduced pressure to give a residue, which was purified by prep-TLC (SiO₂, petroleum ether/EtOAc=2:1) to give S4 (200 mg) as a yellow solid used directly in the next step. To a solution of S4 (100 mg) in dichloromethane (1 mL) was added HCl/dioxane (4 M, 1 mL). and the resulting mixture was stirred at 25° C. for 1 hour. Upon completion, the reaction mixture was partitioned between ethyl acetate (40 mL) and brine (30 mL). The water layer was extracted with i-PrOH/CHCl₃ (3:7, 5×20 mL), then the organic phase was dried over sodium sulfate, filtered, and concentrated under reduced pressure to give S5 (90.0 mg, 51% yield over two steps) as an off-white solid.

LC-MS m/z calculated for C₁₉H₁₇BrN₃O [M+H]⁺ 382.1. Found 382.1.

### (2S,3S)-1-acryloyl-3-(4-bromophenyl)-N-(86uinoline-8-yl) azetidine-2-carboxamide (MY-3A) (3)

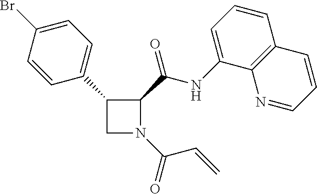 [SMILES: C=CC(=O)N1C[C@H](c2ccc(Br)cc2)[C@H]1C(=O)Nc1cccc2cccnc12]

To a solution of S5(80.0 mg, 209 μmol) in dichloromethane (1 mL) were added triethylamine (42.4 mg, 419 μmol) and acryloyl chloride (37.9 mg, 419 μmol). The mixture was stirred at 25° C. for 2 hours. Upon completion, the reaction mixture was concentrated under reduced pressure. The residue was purified by preparative TLC (SiO₂, petroleum ether/EtOAc=2:1) to give MY-3A (34.0 mg, 37% yield) as a white solid. HRMS ESI-TOF m/z calculated for C₂₂H₁₉BrN₃O₂ [M+H]⁺ 436.0655. Found 436.0649. ¹H NMR (400 MHz, CDCl₃): δ 11.13-10.67 (m, 1H), 8.95-8.84 (m, 1H), 8.83-8.75 (m, 1H), 8.16 (d, J=8.0 Hz, 1H), 7.60-7.50 (m, 4H), 7.45 (dd, J=8.2, 4.2 Hz, 1H), 7.33-7.28 (m, 2H), 6.54 (br d, J=16.9 Hz, 1H), 6.42-6.25 (m, 1H), 5.91-5.75 (m, 1H), 5.18-4.95 (m, 1H), 4.76-4.55 (m, 1H), 4.43-4.14 (m, 2H).

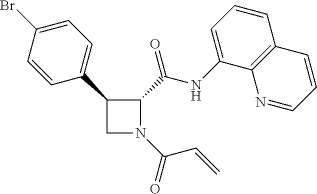 [SMILES: C=CC(=O)N1C[C@@H](c2ccc(Br)cc2)[C@@H]1C(=O)Nc1cccc2cccnc12]

### Synthesis of MY-3B

Prepared in analogous fashion from S1.

(2S,3S)-1-acryloyl-3-(4-bromophenyl)-N-(87uinoline-8-yl) azetidine-2-carboxamide (MY-3B) HRMS ESI-TOF m/z calculated for C₂₂H₁₉BrN₃O₂ [M+H]⁺ 436.0655. Found 436.0643. ¹H NMR (400 MHz, CDCl₃): δ 10.95 (br s, 1H), 8.97-8.69 (m, 2H), 8.15 (d, J=8.2 Hz, 1H), 7.55 (d, J=4.7 Hz, 2H), 7.52 (d, J=8.1 Hz, 2H), 7.45 (dd, J=8.3, 4.2 Hz, 1H), 7.28 (d, J=8.2 Hz, 2H), 6.53 (d, J=16.9 Hz, 1H), 6.41-6.27 (m, 1H), 5.90-5.75 (m, 1H), 5.15-5.02 (m, 1H), 4.73-4.59 (m, 1H), 4.37-4.21 (m, 2H).

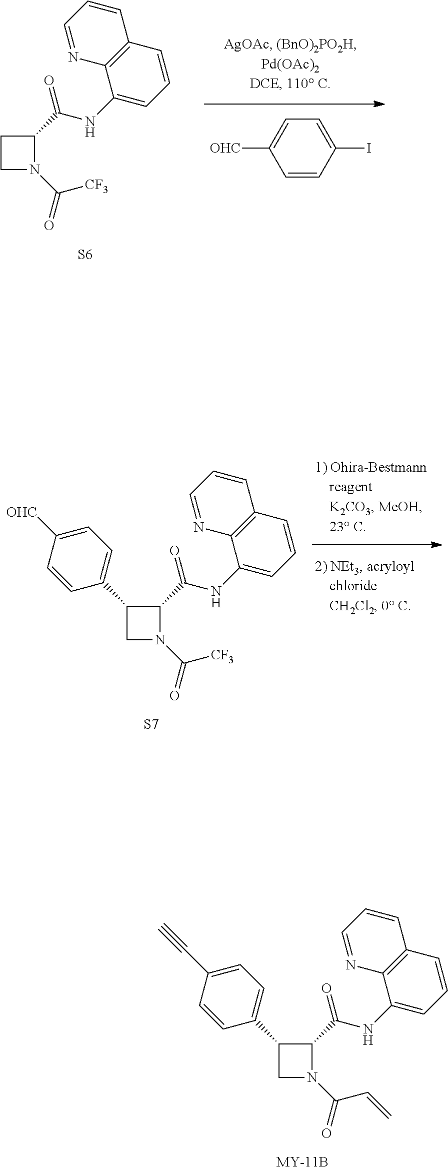

### (2R,3S)-3-(4-formylphenyl)-N-(88uinoline-8-yl)-1-(2,2,2-trifluoroacetyl) azetidine-2-carboxamide (S7)

In a dry flask, S6 (2.42 g, 7.49 mmol, 1.0 equiv.) was dissolved in dry DCE (7.5 mL). To the flask were added AgOAc (2.50 g, 15.0 mmol, 2.0 equiv.), (BnO)₂PO₂H (625 mg, 2.25 mmol, 0.3 equiv.), Pd(Oac)₂ (252 mg, 1.12 mmol, 0.15 equiv.), and 4-iodobenzaldehyde (5.20 g, 22.4 mmol, 3.0 equiv.). The reaction was heated to 110° C. under nitrogen atmosphere for 36 h. The mixture was then cooled to ambient temperature, concentrated and directly purified by flash chromatography (SiO₂, Hexanes/EtOAc=3:1 to 1:1) to yield S7 as a yellow oil (1.28 g, 40%).

¹H NMR (600 MHz, CDCl₃) δ 10.12 (s, 1H), 9.83 (s, 1H), 9.81 (s, 0.5H), 9.74 (s, 0.5H), 8.78 (d, J=3.3 Hz, 1H), 8.76 (d, J=3.6 Hz, 0.5H), 8.37 (d, J=7.5 Hz, 1.5H), 8.15 (dd, J=8.2, 1.3 Hz, 1.5H), 7.68 (d, J=8.1 Hz, 2H), 7.60 (d, J=8.0 Hz, 10H), 7.56 (d, J=8.1 Hz, 3H), 7.52 (d, J=8.0 Hz, 1H), 7.51-7.45 (m, 4H), 7.42 (q, J=8.5, 8.1 Hz, 2H), 7.38 (d, J=3.9 Hz, 1H), 5.70 (d, J=8.2 Hz, 0.5H), 5.51 (d, J=9.8 Hz, 1H), 5.12-5.03 (m, 0.4H), 4.99-4.92 (m, 1H), 4.86 (t, J=9.2 Hz, 1H), 4.74 (d, J=7.5 Hz, 0.5H), 4.64-4.52 (m, 2H), 4.28-4.07 (m, 0.5H).

HRMS (ESI+) m/z calculated for C₂₂H₁₇F₃N₃O₃ [M+H]⁺ 428.1217. Found 428.1219.

### (2R,3S)-1-acryloyl-3-(4-ethynylphenyl)-N-(88uinoline-8-yl) azetidine-2-carboxamide (MY-11B)

In a 1-dram vial, S7(160 mg, 0.37 mmol, 1.0 equiv.) was dissolved in methanol (3 mL). K₂CO₃(77.0 mg, 0.56 mmol, 1.5 equiv.) was then added, followed by Ohira-Bestmann reagent (83.5 μL, 0.56 mmol, 1.5 equiv.). The reaction was allowed to stir at 23° C. for 24 h, then quenched with saturated NaHCO₃(aq), extracted with ethyl acetate (6 times). The combined organic phase was then washed with brine, dried with Na₂SO₄, concentrated, and used directly in the next step.

The crude mixture was dissolved in dichloromethane (1 mL), then triethylamine (0.10 mL, 0.75 mmol, 2.0 equiv.) and acryloyl chloride (40.3 mg, 0.44 mmol, 1.2 equiv.) were added at 0° C. The mixture was stirred at 0° C. for 0.5 hours. Upon completion, the reaction mixture was concentrated under reduced pressure and purified by prep-TLC to give MY-11B (27.0 mg, 22% yield) as a white solid.

¹H NMR (600 MHz, CDCl₃) δ 10.47 (s, 1H), 8.89-8.68 (m, 1H), 8.40 (d, J=7.3 Hz, 1H), 8.14 (d, J=7.9 Hz, 1H), 7.54-7.39 (m, 3H), 7.34 (d, J=7.9 Hz, 2H), 7.27-7.20 (m, 2H), 6.52 (m, 1H), 6.30 (m, 1H), 5.79 (m, 1H), 5.37 (d, J=9.8 Hz, 1H), 4.64 (t, J=9.3 Hz, 1H), 4.51 (s, 1H), 4.31 (s, 1H), 2.97 (s, 1H).

¹³C NMR (151 MHz, CDCl₃) δ 167.87, 165.59, 148.44, 138.36, 136.51, 137.12, 132.78, 132.21, 129.37, 128.07, 127.89, 127.26, 125.71, 122.41, 121.64, 121.51, 117.02, 83.19, 77.55, 69.01, 67.24, 53.46, 51.87, 38.18.

Note: Certain peaks in NMR are broad and split due to the presence of rotamers.

HRMS (ESI+) m/z calcd for C₂₄H₂₀N₃O₂⁺ [M+H]⁺: 382.1550, found: 382.1564.

The enantiomer MY-11A was prepared in the same way from ent-S7.

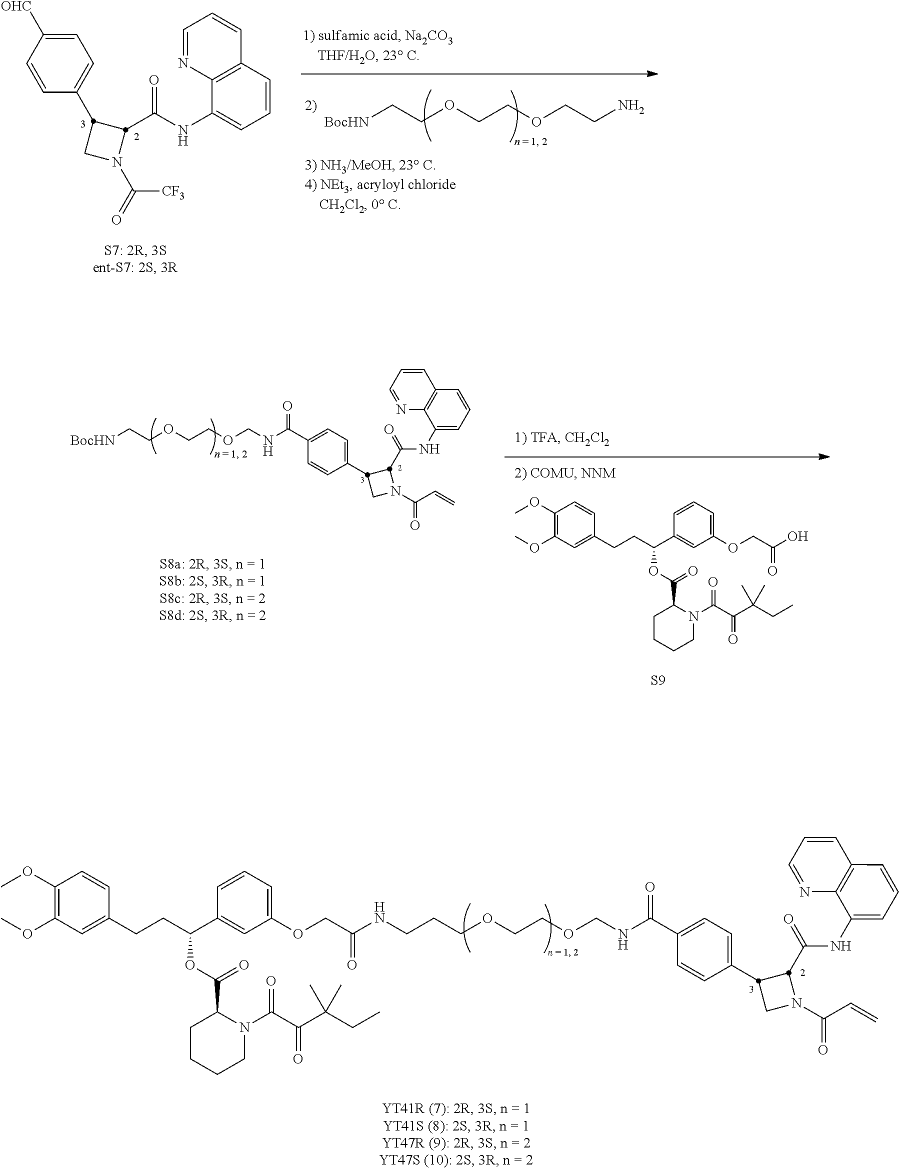

### General Procedure for the Synthesis of Heterobifunctional Compounds

S7 or ent-S7(1.0 equiv.) was dissolved in THE, into which sulfamic acid (2.0 equiv.) was added. The mixture was cooled to 0° C., and an aqueous solution of Na₂CO₃(4.0 equiv.) was added dropwise with vigorous stirring over 15 min. The reaction was further stirred for 1 h at 23° C. until complete consumption of the limiting starting material by TLC. The mixture was then extracted with ethyl acetate (3 times), washed with Na₂S203 (aq), brine, and dried over Na₂SO₄. It was then concentrated to give a yellow oil which was used in the next step without further purification.

The crude acid was then dissolved in DCM, COMU (1.5 equiv.), DIPEA (3 equiv.) and the linker amine (1.2 equiv.) were added. The reaction was stirred at 23° C. overnight. The mixture was then concentrated and purified by flash column (SiO₂, Hexanes/EtOAc=1:1 to 0:1) to give the coupling product (confirmed by LC-MS) which was then used without further characterization. The crude product was then dissolved in methanolic ammonia (7N) and stirred at 23° C. for 3 h until complete conversion by LC-MS. The mixture was concentrated under reduced pressure and dried under high vacuum, then dissolved in dichloromethane. The resulting solution was cooled to 0° C., following which, followed by the addition of triethylamine (3.0 equiv.) and acryloyl chloride (2.0 equiv.) at 0° C. The mixture was stirred at 0° C. for 0.5 hour. Upon completion, the reaction mixture was concentrated under reduced pressure and purified by prep-TLC to give S8a-S8d.

S8a-S8d was dissolved in DCM/TFA (3:1) and stirred at 23° C. for 3 h until LC-MS showed complete conversion of the starting material. The mixture was then concentrated and dried under high vacuum overnight, then dissolved in dichloromethane, followed by the addition of DIPEA (3 equiv.), DMAP (0.1 equiv.), COMU (1.5 equiv.), and S9 (1.1 equiv.). The reaction was stirred at 23° C. overnight. The mixture was concentrated and first purified by prep-TLC, then prep-HPLC to give the final product as a white residue.

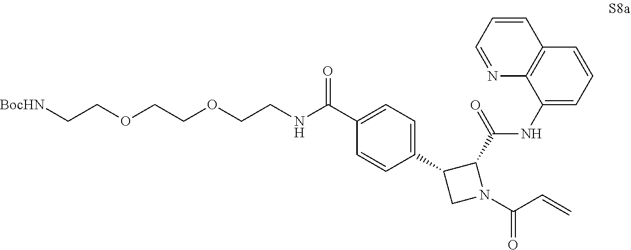 [SMILES: C=CC(=O)N1C[C@H](c2ccc(C(=O)NCCOCCOCCNC(=O)OC(C)(C)C)cc2)[C@@H]1C(=O)Nc1cccc2cccnc12]

¹H NMR (500 MHz, CDCl₃) δ 10.53 (s, 1H), 8.91-8.74 (m, 1H), 8.39 (d, J=7.6 Hz, 1H), 8.24-8.09 (m, 1H), 7.75-7.55 (m, 2H), 7.44 (dt, J=30.4, 7.9 Hz, 5H), 6.63-6.48 (m, 2H), 6.36 (br, 1H), 5.79 (br, 1H), 5.42 (d, J=9.8 Hz, 1H), 5.00 (s, 1H), 4.67 (t, J=9.2 Hz, 1H), 4.56 (s, 1H), 4.36 (d, J=11.0 Hz, 1H), 3.67-3.54 (m, 8H), 3.49 (s, 2H), 3.26 (q, J=5.6 Hz, 2H), 1.43 (s, 9H).

¹³C NMR (126 MHz, CDCl₃) δ 167.81, 166.83, 165.58, 155.98, 148.61, 140.20, 138.49, 136.36, 136.34, 133.79, 129.43, 128.22, 127.88, 127.21, 127.13, 125.70, 122.32, 121.70, 116.82, 79.33, 70.22, 70.18, 70.08, 69.77, 54.03, 51.83, 40.21, 39.66, 38.10, 28.35. HRMS (ESI+) m z calcd for C₃₄H₄₂N₅O₇⁺ [M+H]⁺: 632.3079, found: 632.3091. The enantiomer S8b was prepared in the same way from ent-S7.

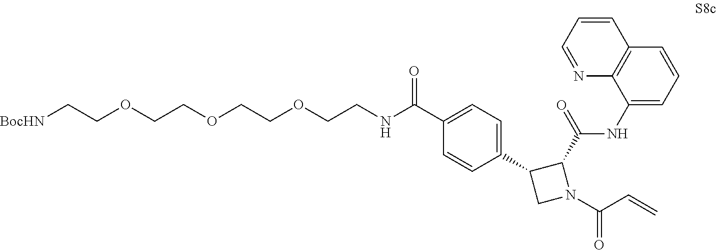 [SMILES: C=CC(=O)N1C[C@H](c2ccc(C(=O)NCCOCCOCCOCCNC(=O)OC(C)(C)C)cc2)[C@@H]1C(=O)Nc1cccc2cccnc12]

¹H NMR (600 MHz, CD₃OD) δ 8.84 (dd, J=4.2, 1.6 Hz, 1H), 8.29-8.14 (m, 2H), 7.63 (d, J=7.9 Hz, 2H), 7.58-7.47 (m, 4H), 7.39 (t, J=8.0 Hz, 1H), 6.65 (dd, J=17.2, 10.5 Hz, 0.5H), 6.48-6.30 (m, 1.2H), 5.93 (d, J=10.5 Hz, 0.5H), 5.82-5.68 (m, 0.6H), 5.50 (d, J=9.8 Hz, 0.5H), 4.75 (dd, J=31.2, 8.3 Hz, 1.1H), 4.59-4.42 (m, 1.6H), 3.61-3.54 (m, 8H), 3.50 (s, 2H), 3.49-3.45 (m, 2H), 3.45-3.41 (m, 2H), 3.34-3.32 (m, 1H), 3.18 (t, J=5.6 Hz, 2H), 1.43 (s, 9H). (Some peaks are broad and split due to the presence of rotamers.)

¹³C NMR (151 MHz, CD₃OD) δ 168.15, 167.64, 166.26, 157.00, 148.71, 140.26, 138.52, 136.20, 133.28, 133.03, 128.11, 128.06, 127.32, 126.93, 126.43, 125.97, 122.64, 122.37, 121.73, 117.14, 116.53, 78.66, 70.11, 70.09, 69.80, 69.75, 69.59, 69.05, 67.13, 53.62, 39.85, 39.44, 37.43, 27.40.

HRMS (ESI+) m z calcd for C₃₆H₄₆N₅O₈+ [M+H]⁺: 676.3341, found: 676.3352.

The enantiomer S8d was prepared in the same way from ent-S7.

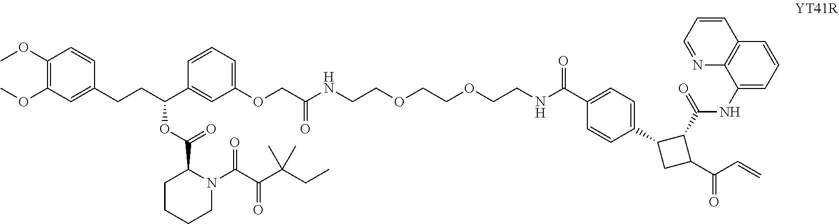 [SMILES: C=CC(=O)C1C[C@H](c2ccc(C(=O)NCCOCCOCCNC(=O)COc3cccc([C@@H](CCc4ccc(OC)c(OC)c4)OC(=O)[C@@H]4CCCCN4C(=O)C(=O)C(C)(C)CC)c3)cc2)[C@@H]1C(=O)Nc1cccc2cccnc12]

¹H NMR (500 MHz, CDCl₃) § 10.53 (s, 1H), 8.82 (s, 1H), 8.39 (d, J=7.1 Hz, 1H), 8.14 (d, J=8.0 Hz, 1H), 7.59 (d, J=7.3 Hz, 2H), 7.48 (t, J=10.4 Hz, 4H), 7.40 (t, J=7.8 Hz, 1H), 7.32-7.30 (m, 1H), 7.01 (t, J=7.9 Hz, 2H), 6.93 (s, 1H), 6.86-6.77 (m, 2H), 6.70 (d, J=8.7 Hz, 2H), 6.63-6.48 (m, 2H), 5.85-5.72 (m, 2H), 5.42 (d, J=9.8 Hz, 1H), 5.32 (d, J=4.8 Hz, 1H), 4.67 (t, J=9.2 Hz, 1H), 4.48 (s, 3H), 4.36 (s, 1H), 3.91-3.84 (m, 6H), 3.61-3.53 (m, 8H), 3.52-3.48 (m, 2H), 3.39 (d, J=12.9 Hz, 1H), 3.19 (t, J=12.2 Hz, 1H), 2.60 (ddt, J=29.7, 15.2, 6.9 Hz, 2H), 2.38 (d, J=13.4 Hz, 1H), 2.25 (dt, J=9.0, 4.6 Hz, 1H), 2.06 (dt, J=14.3, 7.3 Hz, 1H), 1.87-1.61 (m, 6H), 1.44 (dq, J=60.5, 12.8 Hz, 3H), 1.23 (d, J=7.7 Hz, 6H), 1.14 (s, 1H), 0.90 (t, J=7.3 Hz, 3H).

¹³C NMR (126 MHz, CDCl₃) δ 207.86, 169.71, 168.20, 168.13, 167.93, 167.32, 165.57, 157.38, 148.92, 148.71, 147.41, 141.90, 138.61, 136.24, 133.76, 133.34, 131.21, 130.06, 129.49, 128.23, 127.87, 127.21, 127.07, 125.68, 121.74, 120.16, 120.06, 117.21, 116.70, 114.15, 113.37, 111.75, 111.34, 76.51, 70.22, 70.19, 69.71, 69.62, 67.38, 55.94, 55.86, 51.28, 46.71, 44.16, 39.64, 39.53, 38.73, 38.61, 38.20, 32.48, 31.27, 26.43, 24.94, 23.56, 23.47, 23.17, 21.20, 21.03, 8.82, 8.77.

HRMS (ESI+) m z calcd for C₆₁H₇₃N₆O₁₃⁺ [M+H]⁺: 1097.5230, found: 1097.5238.

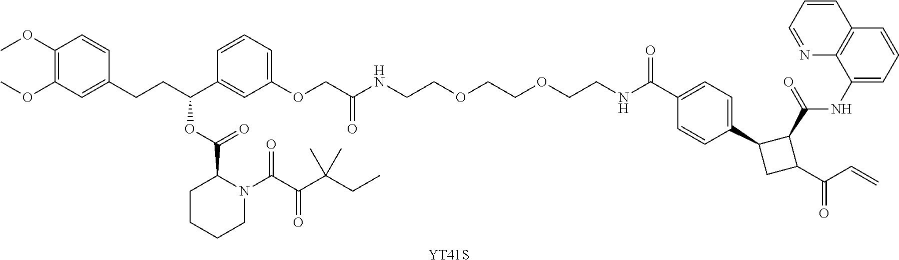 [SMILES: C=CC(=O)C1C[C@@H](c2ccc(C(=O)NCCOCCOCCNC(=O)COc3cccc([C@@H](CCc4ccc(OC)c(OC)c4)OC(=O)[C@@H]4CCCCN4C(=O)C(=O)C(C)(C)CC)c3)cc2)[C@H]1C(=O)Nc1cccc2cccnc12 ; "YT41S"]

¹H NMR (600 MHz, CD₃OD) δ 8.83 (s, 1H), 8.24 (d, J=7.9 Hz, 2H), 7.65-7.46 (m, 7H), 7.38 (t, J=7.8 Hz, 1H), 7.29 (t, J=7.8 Hz, 1H), 7.04-6.95 (m, 2H), 6.90 (d, J=7.8 Hz, 1H), 6.85 (d, J=8.0 Hz, 1H), 6.79 (d, J=12.5 Hz, 1H), 6.71 (d, J=7.9 Hz, 1H), 6.48-6.31 (m, 0.6H), 5.98-5.65 (m, 1.4H), 5.55-5.43 (m, 2H), 4.55-4.41 (m, 4H), 3.80 (s, 3H), 3.79 (s, 3H), 3.52 (d, J=17.0 Hz, 8H), 3.42 (s, 3H), 3.41 (s, 3H), 3.21 (t, J=12.9 Hz, 1H), 2.59 (m, 2H), 2.40-2.18 (m, 2H), 2.11-1.99 (m, 1H), 1.70 (m, 5H), 1.53-1.27 (m, 4H), 1.22 (d, J=6.0 Hz, 6H), 1.12 (s, 1H), 0.89 (t, J=7.3 Hz, 3H).

¹³C NMR (151 MHz, CD₃OD) δ 207.65, 169.62, 169.60, 169.58, 167.70, 157.87, 149.01, 148.70, 147.43, 146.45, 141.96, 136.19, 133.80, 133.26, 133.01, 129.62, 128.11, 128.05, 126.89, 126.42, 125.91, 121.71, 120.30, 119.68, 119.58, 118.92, 114.17, 112.92, 112.17, 111.83, 76.56, 69.85, 69.07, 69.03, 66.88, 56.87, 55.14, 55.04, 53.41, 51.42, 46.30, 44.30, 39.38, 38.48, 37.86, 32.26, 32.18, 30.85, 27.20, 25.95, 25.67, 24.49, 24.27, 22.68, 22.42, 22.36, 22.25, 20.75, 20.66, 7.72.

HRMS (ESI+) m z calcd for C₆₁H₇₃N₆O₁₃⁺ [M+H]⁺: 1097.5230, found: 1097.5237.

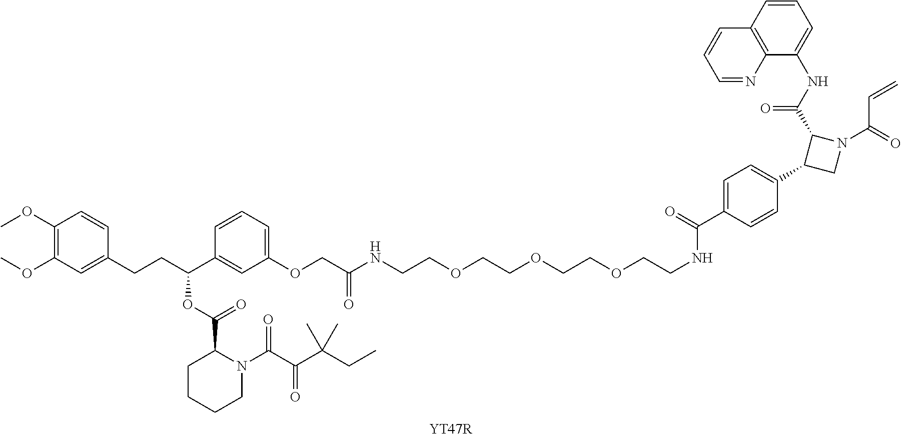 [SMILES: C=CC(=O)N1C[C@H](c2ccc(C(=O)NCCOCCOCCOCCNC(=O)COc3cccc([C@@H](CCc4ccc(OC)c(OC)c4)OC(=O)[C@@H]4CCCCN4C(=O)C(=O)C(C)(C)CC)c3)cc2)[C@@H]1C(=O)Nc1cccc2cccnc12 ; "YT47R"]

### (Mixture of Rotamers)

¹H NMR (600 MHz, CD₃OD) δ 8.83 (dd, J=4.2, 1.6 Hz, 1H), 8.30-8.12 (m, 2H), 7.61 (d, J=7.6 Hz, 2H), 7.57-7.46 (m, 4H), 7.38 (t, J=8.0 Hz, 1H), 7.29 (t, J=7.9 Hz, 1H), 7.03-6.95 (m, 2H), 6.91 (dd, J=8.3, 2.3 Hz, 1H), 6.85 (dd, J=8.2, 2.2 Hz, 1H), 6.81-6.75 (m, 1H), 6.73-6.67 (m, 1H), 6.67-6.29 (m, 2H), 6.00-5.67 (m, 2H), 5.54-5.19 (m, 1H), 4.73 (d, J=30.1 Hz, 1H), 4.51 (m, 4H), 3.80 (s, 3H), 3.79 (s, 3H), 3.61-3.46 (m, 13H), 3.46-3.37 (m, 5H), 3.21 (td, J=13.2, 3.0 Hz, 1H), 2.68-2.53 (m, 2H), 2.38-2.20 (m, 2H), 2.11-2.01 (m, 1H), 1.82-1.59 (m, 5H), 1.54-1.43 (m, 1H), 1.42-1.27 (m, 2H), 1.22 (d, J=6.3 Hz, 6H), 1.12 (d, J=1.5 Hz, 1H), 0.88 (t, J=7.5 Hz, 2.5H), 0.80 (t, J=7.5 Hz, 0.5H).

¹³C NMR (151 MHz, CD₃OD) δ 207.64, 207.59, 169.58, 169.57, 168.11, 167.69, 166.99, 166.22, 157.86, 149.02, 148.71, 147.44, 141.97, 141.79, 140.28, 138.50, 136.20, 134.95, 134.42, 133.84, 133.79, 133.27, 133.04, 129.62, 128.06, 126.92, 126.43, 125.96, 121.72, 120.30, 119.67, 119.56, 114.15, 113.00, 112.96, 112.18, 111.83, 76.76, 76.54, 70.11, 70.08, 69.81, 69.79, 69.03, 68.96, 67.10, 66.90, 56.86, 55.14, 55.05, 51.41, 46.30, 44.30, 39.44, 38.75, 38.55, 37.88, 37.74, 37.42, 32.26, 32.18, 30.86, 27.21, 25.95, 24.49, 24.08, 22.69, 22.43, 22.36, 22.26, 20.75, 20.66, 7.79, 7.73.

HRMS (ESI+) m/z calcd for C₆₃H₇₇N₆O₁₄⁺ [M+H]⁺: 1141.5492, found: 1141.5519.

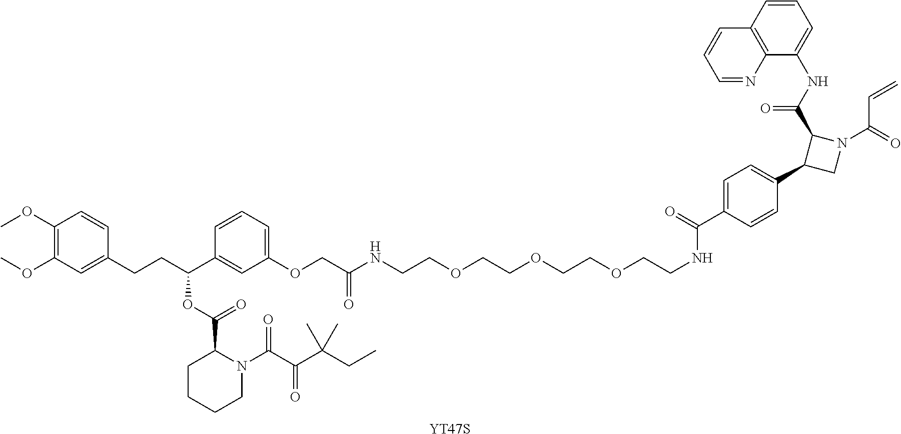 [SMILES: C=CC(=O)N1C[C@@H](c2ccc(C(=O)NCCOCCOCCOCCNC(=O)COc3cccc([C@@H](CCc4ccc(OC)c(OC)c4)OC(=O)[C@@H]4CCCCN4C(=O)C(=O)C(C)(C)CC)c3)cc2)[C@H]1C(=O)Nc1cccc2cccnc12 ; "YT47S"]

¹H NMR (600 MHz, CDCl₃) δ 10.52 (s, 1H), 8.81 (s, 1H), 8.39 (d, J=6.7 Hz, 1H), 8.15 (d, J=7.9 Hz, 1H), 7.60 (d, J=6.8 Hz, 2H), 7.52-7.38 (m, 4H), 7.29 (d, J=6.2 Hz, 1H), 7.06 (d, J=5.4 Hz, 1H), 6.99 (dd, J=13.0, 7.8 Hz, 1H), 6.95-6.89 (m, 1H), 6.85 (d, J=8.2 Hz, 1H), 6.80 (dd, J=8.4, 4.5 Hz, 1H), 6.75-6.65 (m, 3H), 6.53 (s, 1H), 5.77 (q, J=5.8 Hz, 1H), 5.42 (d, J=9.8 Hz, 1H), 4.66 (t, J=9.2 Hz, 1H), 4.49 (s, 2H), 4.37 (s, 1H), 3.87 (d, J=6.9 Hz, 6H), 3.62-3.51 (m, 17H), 3.39 (d, J=12.9 Hz, 1H), 3.19 (td, J=13.1, 2.6 Hz, 1H), 2.58 (m, 2H), 2.38 (d, J=13.7 Hz, 1H), 2.29-2.20 (m, 1H), 2.10-2.04 (m, 1H), 1.83-1.61 (m, 6H), 1.57-1.43 (m, 1H), 1.40-1.30 (m, 1H), 1.23 (d, J=10.1 Hz, 6H), 1.14 (d, J=4.6 Hz, 1H), 0.90 (t, J=7.4 Hz, 3H).

¹³C NMR (151 MHz, CDCl₃) δ 207.87, 169.69, 168.13, 167.31, 166.80, 166.53, 165.58, 157.41, 148.90, 148.60, 147.38, 141.86, 138.47, 136.33, 133.78, 133.34, 130.04, 129.43, 128.20, 127.89, 127.26, 127.12, 125.68, 121.72, 120.15, 120.06, 116.54, 116.39, 114.25, 113.30, 111.72, 111.32, 76.51, 70.47, 70.23, 70.20, 69.75, 69.64, 67.39, 56.69, 55.93, 55.85, 53.46, 51.27, 46.71, 44.17, 39.69, 38.85, 38.78, 38.20, 38.03, 32.58, 32.48, 31.27, 26.44, 24.94, 23.55, 23.47, 23.15, 21.22, 8.83, 8.78, 1.92.

HRMS (ESI+) m z calcd for C₆₃H₇₇N₆O₁₄⁺ [M+H]⁺: 1141.5492, found: 1141.5482.

### References

1. Zhang, X.; Luukkonen, L. M.; Eissler, C. L.; Crowley, V. M.; Yamashita, Y.; Schafroth, M. A.; Kikuchi, S.; Weinstein, D. S.; Symons, K. T.; Nordin, B. E.; Rodriguez, J. L.; Wucherpfennig, T. G.; Bauer, L. G.; Dix, M. M.; Stamos, D.; Kinsella, T. M.; Simon, G. M.; Baltgalvis, K. A.; Cravatt, B. F., DCAF11 Supports Targeted Protein Degradation by Electrophilic Proteolysis-Targeting Chimeras. J. Am. Chem. Soc. 2021, 143 (13), 5141-5149.
2. Banchenko, S.; Krupp, F.; Gotthold, C.; Bürger, J.; Graziadei, A.; O'Reilly, F. J.; Sinn, L.; Ruda, O.; Rappsilber, J.; Spahn, C. M., Structural insights into Cullin4-RING ubiquitin ligase remodelling by Vpr from simian immunodeficiency viruses. PLoS Pathog. 2021, 17 (8), e1009775.
   3. Vinogradova, E. V.; Zhang, X.; Remillard, D.; Lazar, D. C.; Suciu, R. M.; Wang, Y.;
Bianco, G.; Yamashita, Y.; Crowley, V. M.; Schafroth, M. A.; Yokoyama, M.; Konrad, D. B.; Lum, K. M.; Simon, G. M.; Kemper, E. K.; Lazear, M. R.; Yin, S.; Blewett, M. M.; Dix, M. M.; Nguyen, N.; Shokhirev, M. N.; Chin, E. N.; Lairson, L. L.; Melillo, B.; Schreiber, S. L.; Forli, S.; Teijaro, J. R.; Cravatt, B. F., An Activity-Guided Map of Electrophile-Cysteine Interactions in Primary Human T Cells. Cell 2020, 182 (4), 1009-1026 e29.
4. Maetani, M.; Zoller, J.; Melillo, B.; Verho, O.; Kato, N.; Pu, J.; Comer, E.; Schreiber, S. L., Synthesis of a Bicyclic Azetidine with In Vivo Antimalarial Activity Enabled by Stereospecific, Directed C(sp(3))—H Arylation. J Am Chem Soc 2017, 139 (32), 11300-11306.

### SCHEME FOR SYNTHESIS OF ADDITIONAL COMPOUNDS

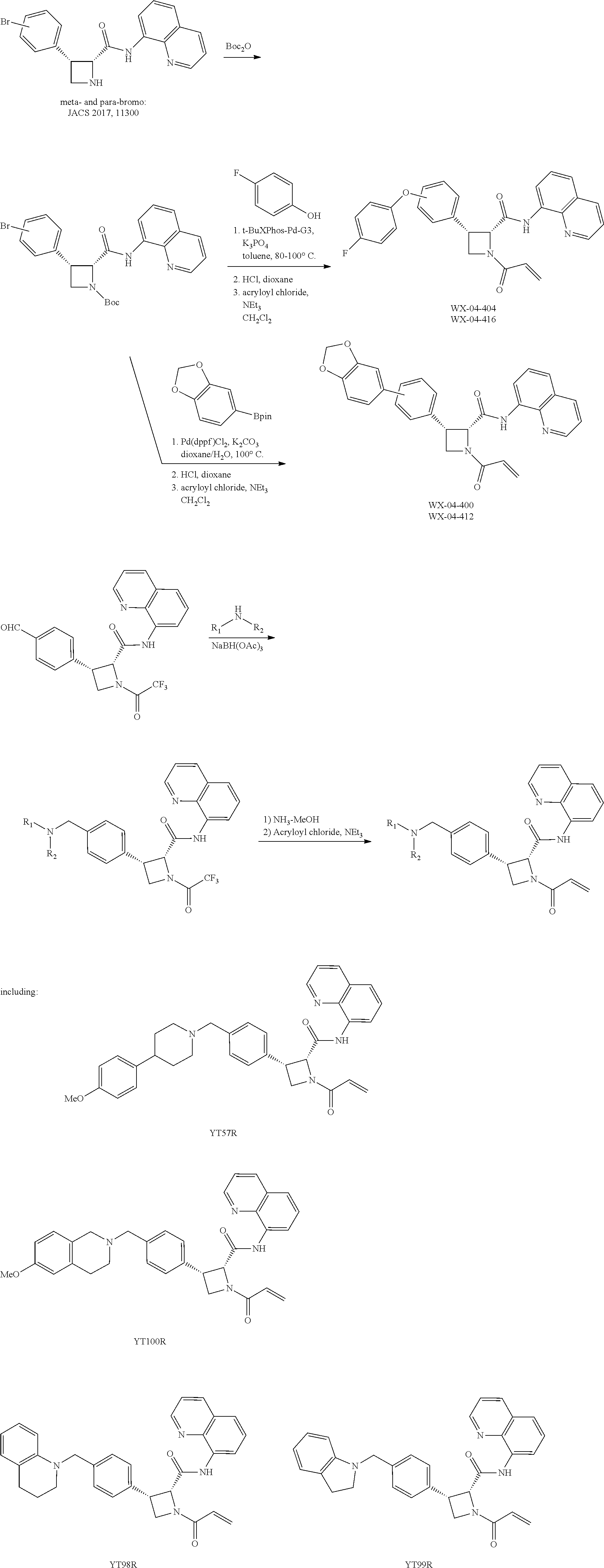

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

1. A bifunctional degrader of Formula (I) 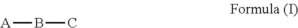
wherein:
A is a ligand to a protein of interest;
B is a linker that is either a bond or a molecular linker that is covalently linked to both A and C; and
C is a ligand to the E3 ligase substrate receptor DCAF1.

2. The bifunctional degrader of claim 1, wherein:
the protein of interest is a protein having the ability to bind to the ligand A;
B is a molecular linker covalently linking both A and C; and
the ligand C can form a covalent bond with a cysteine residue of DCAF1.

3. The bifunctional degrader of claim 2, wherein the ligand C is an azetidinyl acrylamide.

4. The bifunctional degrader of claim 3, wherein the cysteine residue is C1113 of DCAF1, wherein the amino acid numbering is based on DCAF1 Isoform 1 (Accession No. Q9Y4B6-1).

5. The bifunctional degrader of claim 3, wherein the cysteine residue is C1112 of DCAF1, wherein the amino acid numbering is based on DCAF1 Isoform 2 (Accession No. Q9Y4B6-2).

6. The bifunctional degrader of claim 3, wherein the cysteine residue is C664 of DCAF1, wherein the amino acid numbering is based on DCAF1 Isoform 3 (Accession No. Q9Y4B6-3).

7. The bifunctional degrader of claim 3 wherein the azetidine acrylamide forms a covalent link to the cysteine residue of DCAF1 via a Michael addition reaction.

8. The bifunctional degrader of claim 1, wherein the protein of interest is selected from the group consisting of FKBP12, BRD4, an androgen receptor, an estrogen receptor, IRAK4, a JAK protein, BCL-XL, BCL-2, and Stat3.

9. The bifunctional degrader of claim 3, wherein the azetidinyl acrylamide has the structure of Formula C-1: 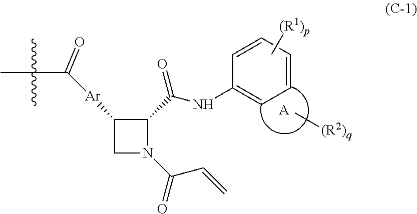
wherein:
Ar is a C₆-C₁₀ aryl, optionally substituted with 1-3 moieties selected from the group consisting of: halo, hydroxy, cyano, optionally substituted C₁-C₆ alkyl, —O—(C₁-C₆ alkyl), optionally substituted C₆-C₁₀ aryl, optionally substituted C₃-C₈ cycloalkyl, —C(═O)—(C₁-C₆ alkyl), optionally substituted-(C₁-C₃)ₙ-heterocyclylphenyl, C₆-C₁₀ aryl, —(C₁-C₃)ₙ-linked optionally substituted five- to six-membered heterocyclyl, —(C₁-C₃)ₙ-linked optionally substituted five- to six-membered heterocyclyl fused to an optionally substituted C₆-C₁₀ aryl, and —(C₁-C₃)ₙ-linked optionally substituted five- to six-membered heteroaryl;
each R¹ independently is an optional substituent selected from the group consisting of: halo, cyano, optionally substituted C₁-C₆ alkyl, —O—C₁-C₆ alkyl, optionally substituted C₆-C₁₀ aryl, optionally substituted C₃-C₈ cycloalkyl, —C(═O)(C₁-C₆ alkyl), optionally substituted five- to six-membered heterocyclyl, and optionally substituted five- to six-membered heteroaryl;
n is 0, 1, or 2;
p is 0, 1 or 2;
ring A is a five- or six-membered heteroaryl;
each R² independently is an optional substituent selected from the group consisting of: halo, cyano, optionally substituted C₁-C₆ alkyl, —O—C₁-C₆ alkyl, optionally substituted C₆-C₁₀ aryl, optionally substituted C₃-C₈ cycloalkyl, —C(═O)(C₁-C₆ alkyl), optionally substituted five- to six-membered heterocyclyl, and optionally substituted five- to six-membered heteroaryl;
q is 0, 1, or 2; and [symbol]
indicates the point of attachment to the linker B.

10. The bifunctional degrader of claim 9, wherein:
Ar is substituted with 1-2 substituents selected from the group consisting of halo, hydroxy, cyano, —O—C₁-C₆alkyl, -alkynylphenyl, flurophenoxy, methoxyphenyl, —((C₁-C₃)ₙ)-4-(4-methoxyphenyl) piperidine and —(C₁-C₃)ₙ-linked optionally substituted five- to six-membered heterocyclyl fused to an optionally substituted C₆-C₁₀ aryl.

11. The bifunctional degrader of claim 10, wherein:
the —(C₁-C₃)ₙ-linked optionally substituted five- to six-membered heterocyclyl fused to an optionally substituted C₆-C₁₀ aryl is selected from the group consisting of —((C₁-C₃)ₙ)-benzo[d][1,3]dioxolyl, —((C₁-C₃)ₙ)-1,2,3,4-tetrahydroquinoline-1-yl, —((C₁-C₃)ₙ)-1,2,3,4-tetrahydroisoquinoline-2-yl, —((C₁-C₃)ₙ)-6-methoxy-1,2,3,4-tetrahydroisoquinoline-2-yl, and —((C₁-C₃)ₙ)-indoline-1-yl; and
n is 0 or 1.

12. The bifunctional degrader of claim 9, wherein Formula (C-1) has the formula (C-1a):

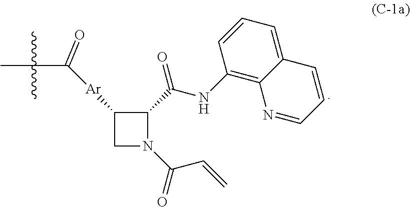

13. The bifunctional degrader of claim 9, wherein 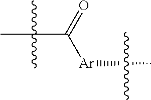
is:

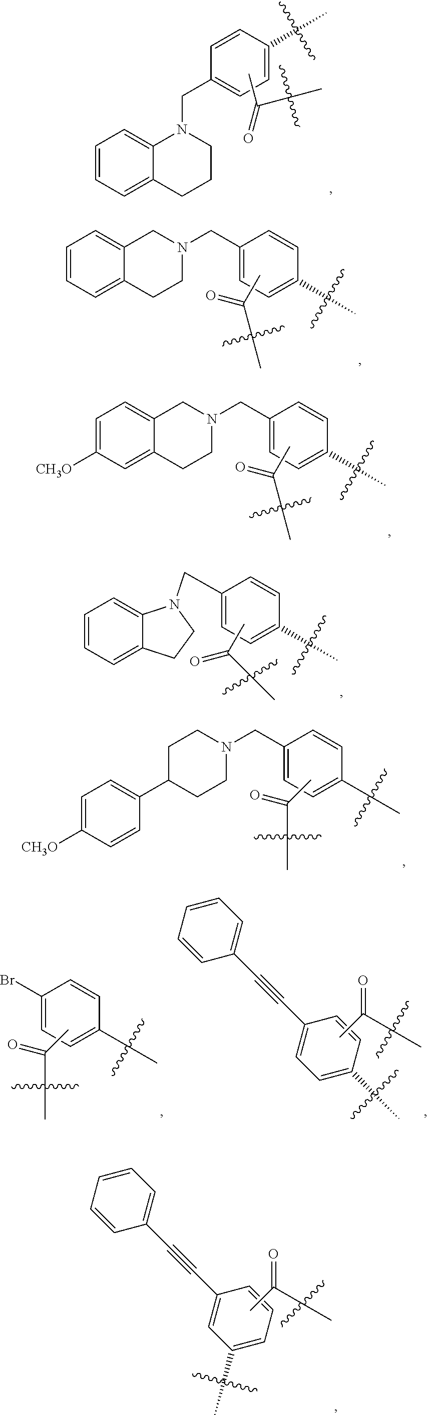

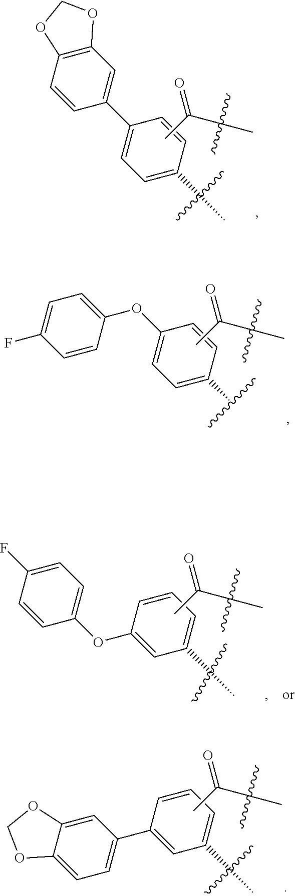

14. The bifunctional degrader of claim 1, wherein linker B comprises a moiety having ethylene repeat units, the moiety having the formula (B-1) 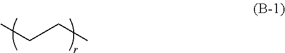
wherein r is an integer from 1 to 10; or a moiety having ethylene glycol repeat units, the moiety having the formula (B-2) 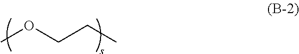
wherein s is an integer from 1 to 10.

15. The bifunctional degrader of claim 1, wherein linker B is

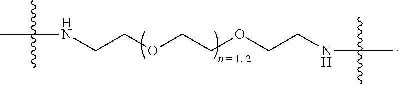

16. The bifunctional degrader of claim 1, wherein linker B is selected from the group consisting of:

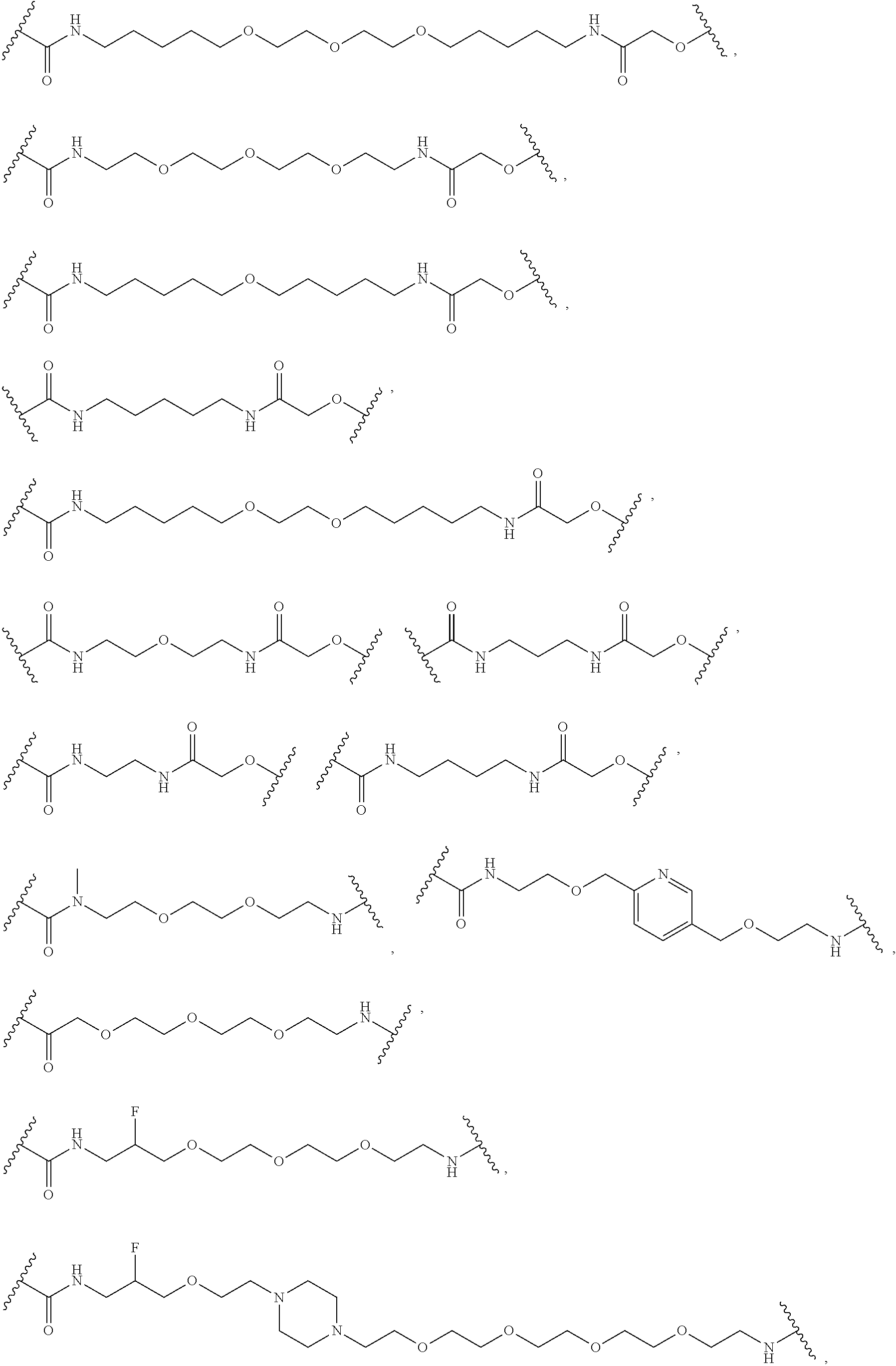

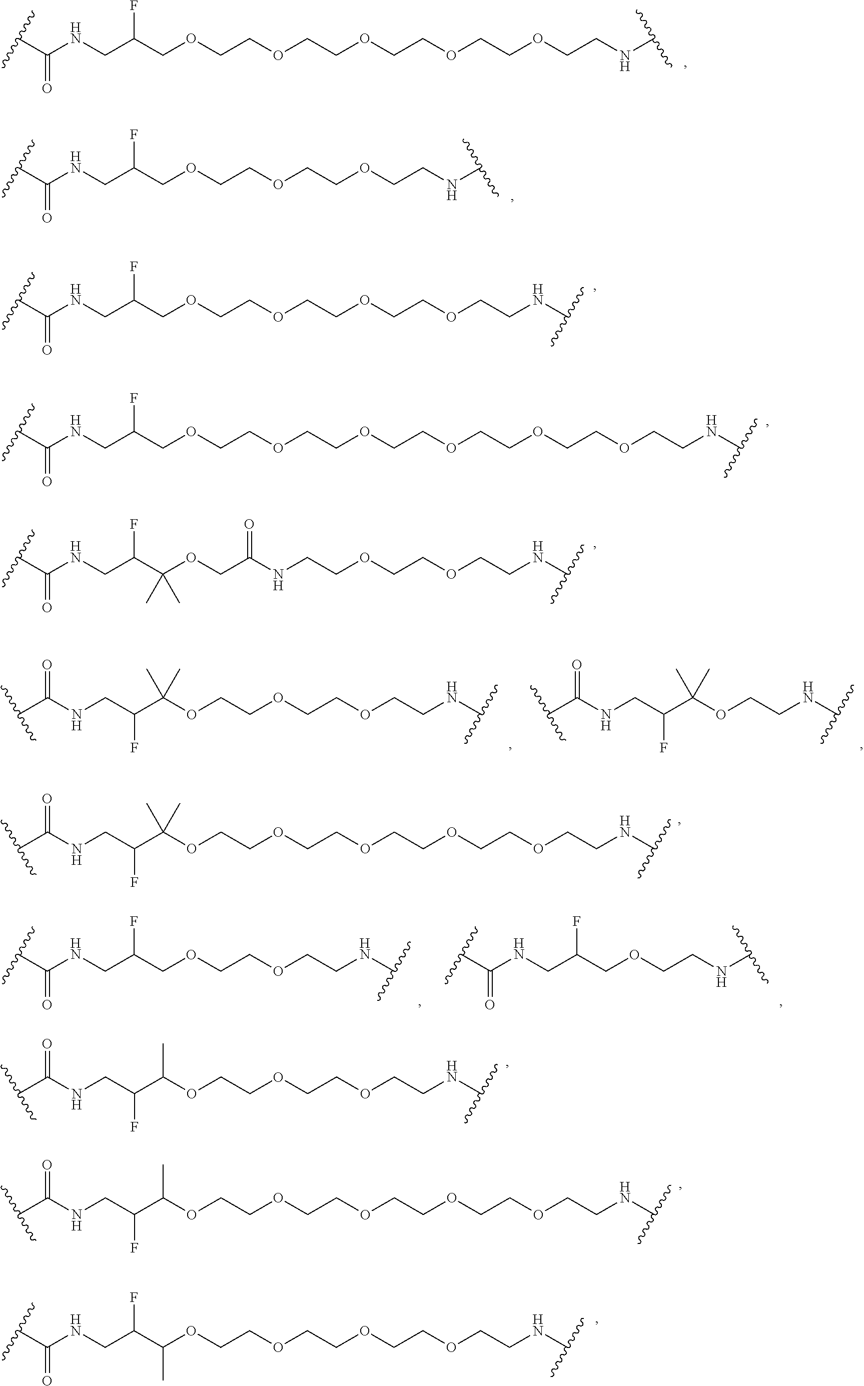

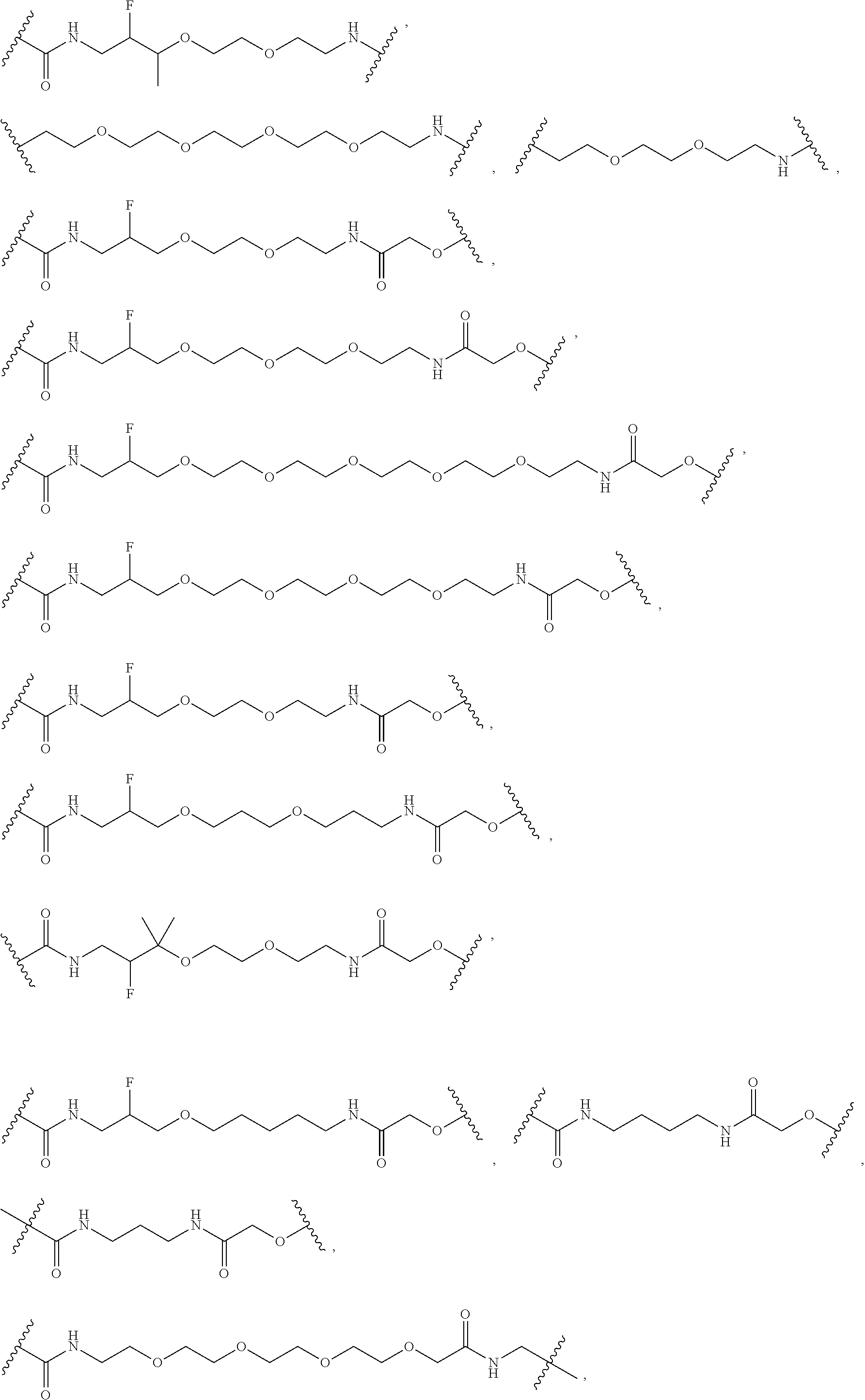

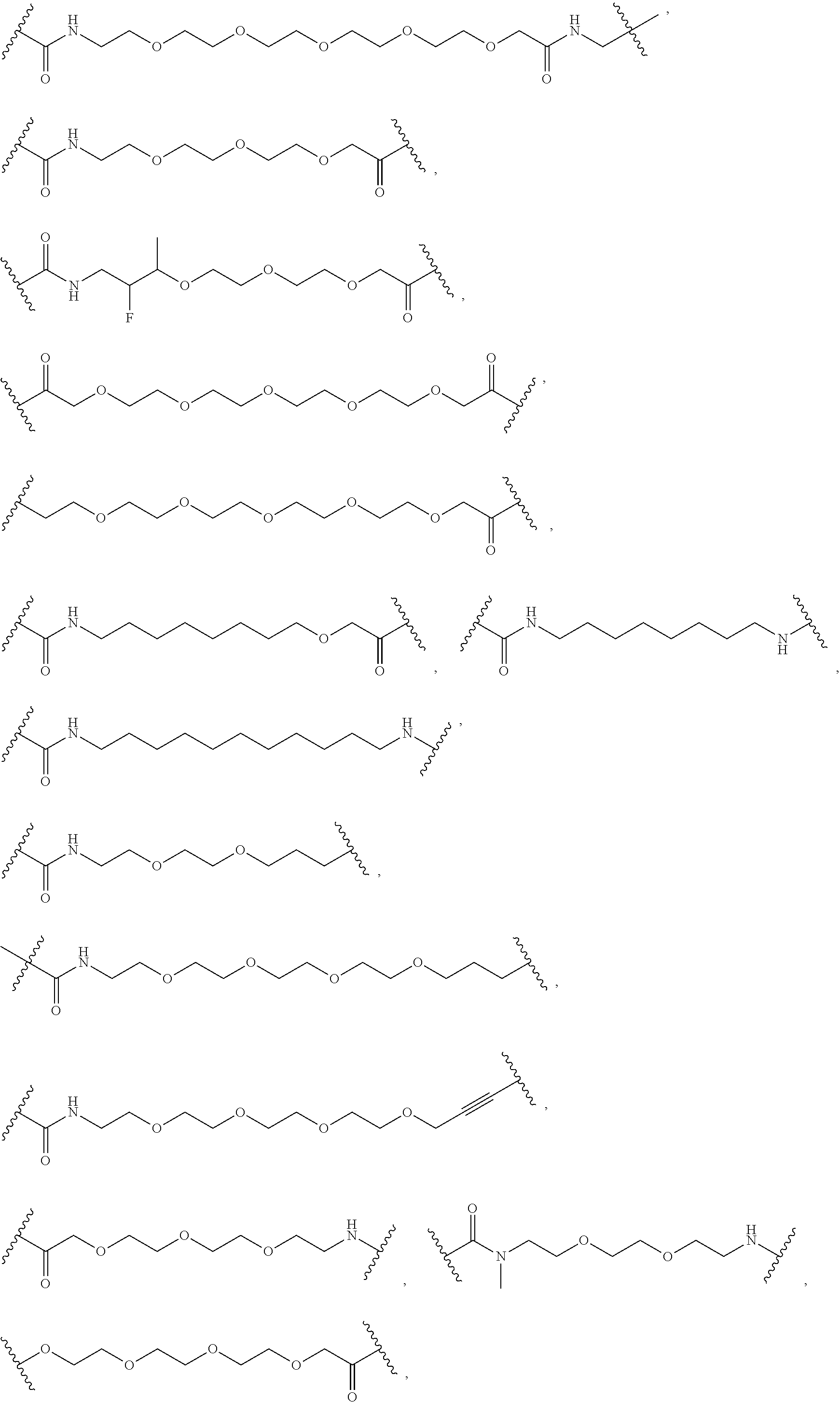

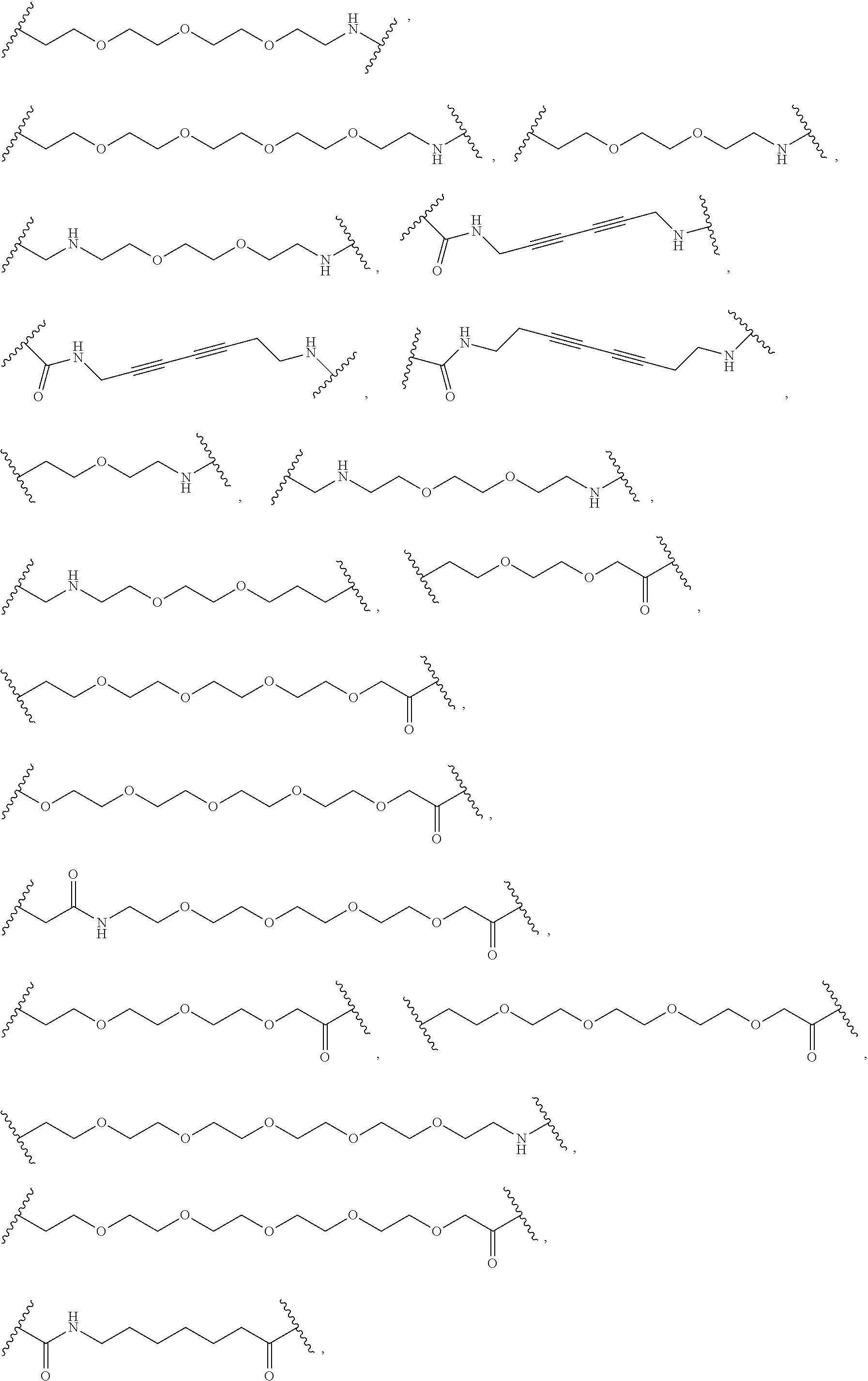

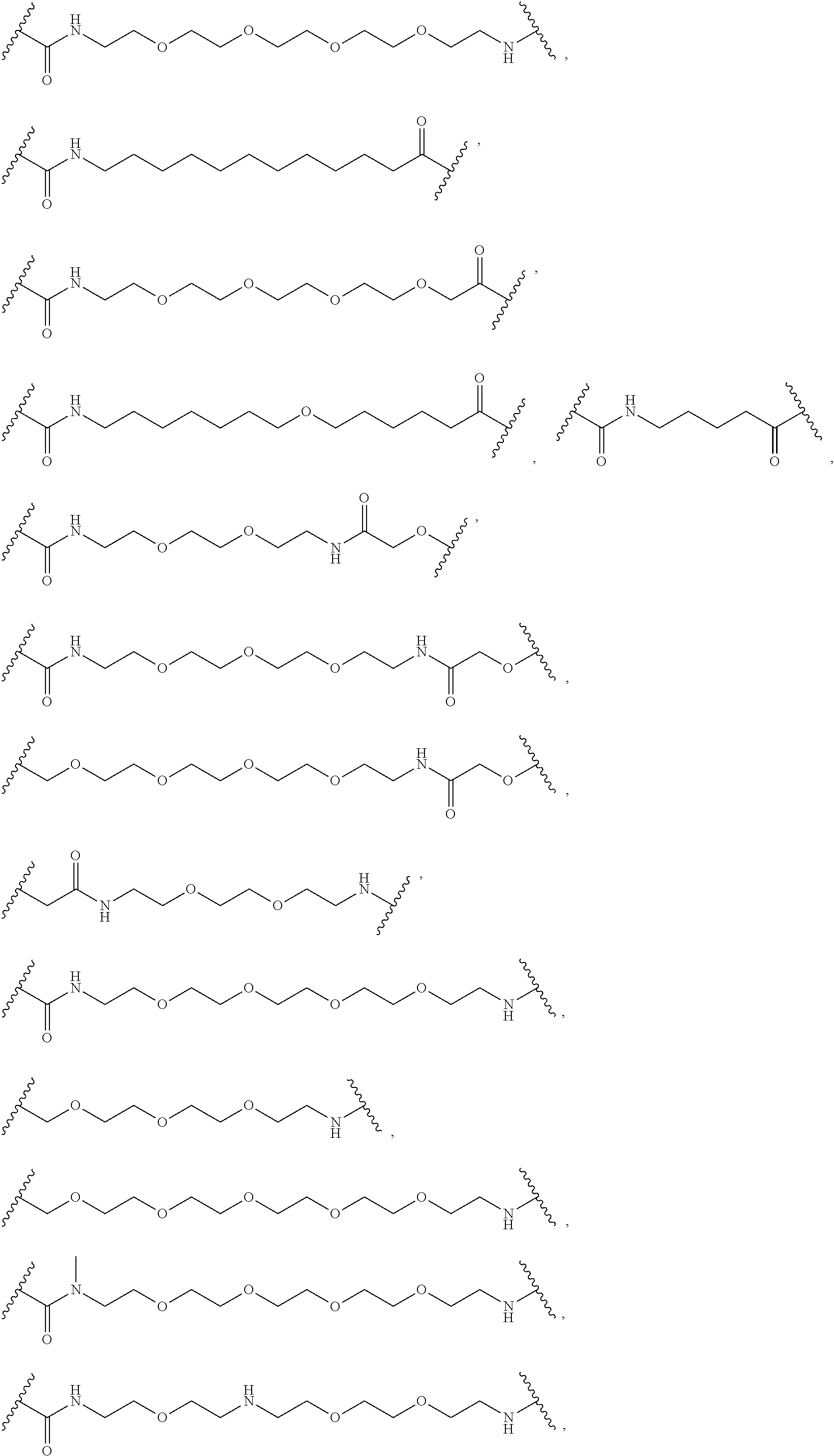

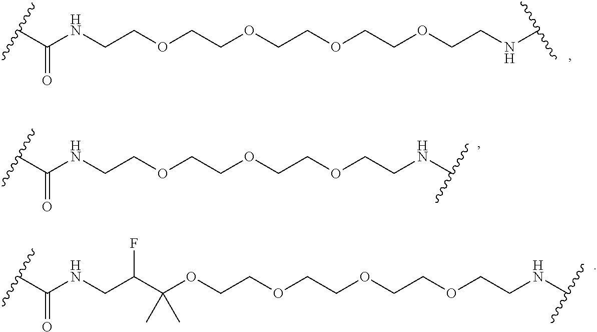 [SMILES: *NCCOCCOCCOCCOCCNC(*)=O ; *NCCOCCOCCOCCNC(*)=O ; *NCCOCCOCCOCCOC(C)(C)C(F)CNC(*)=O]

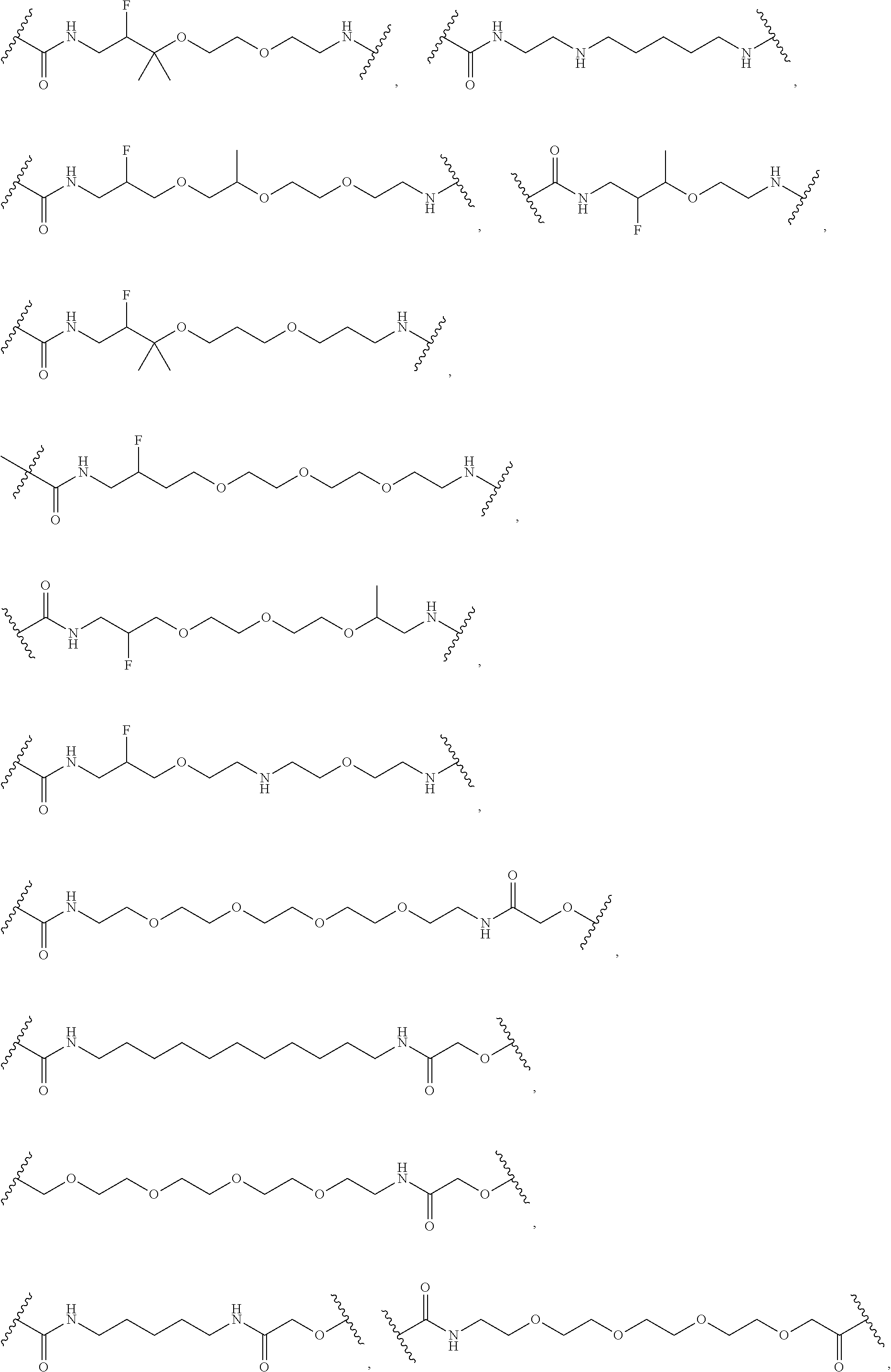

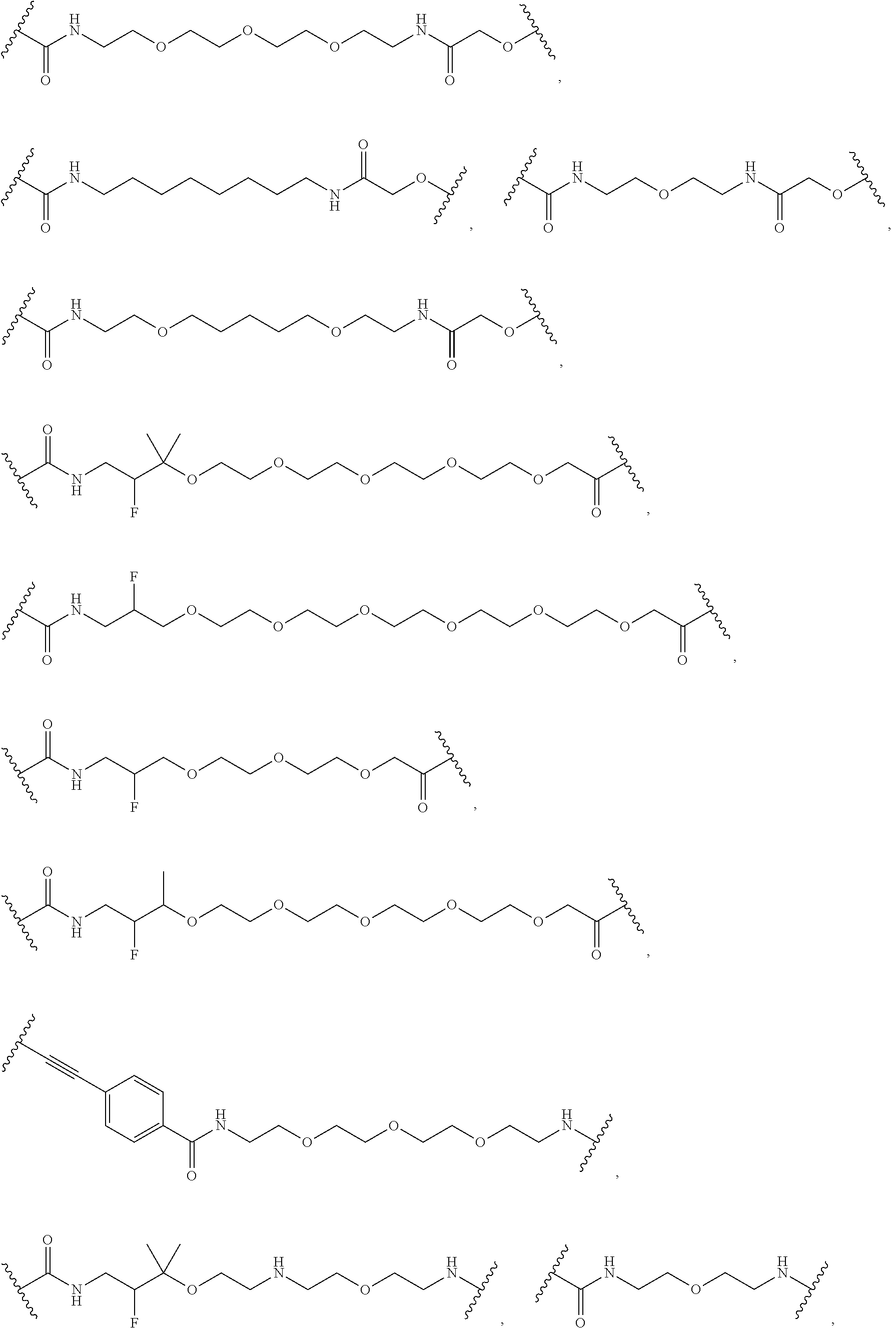

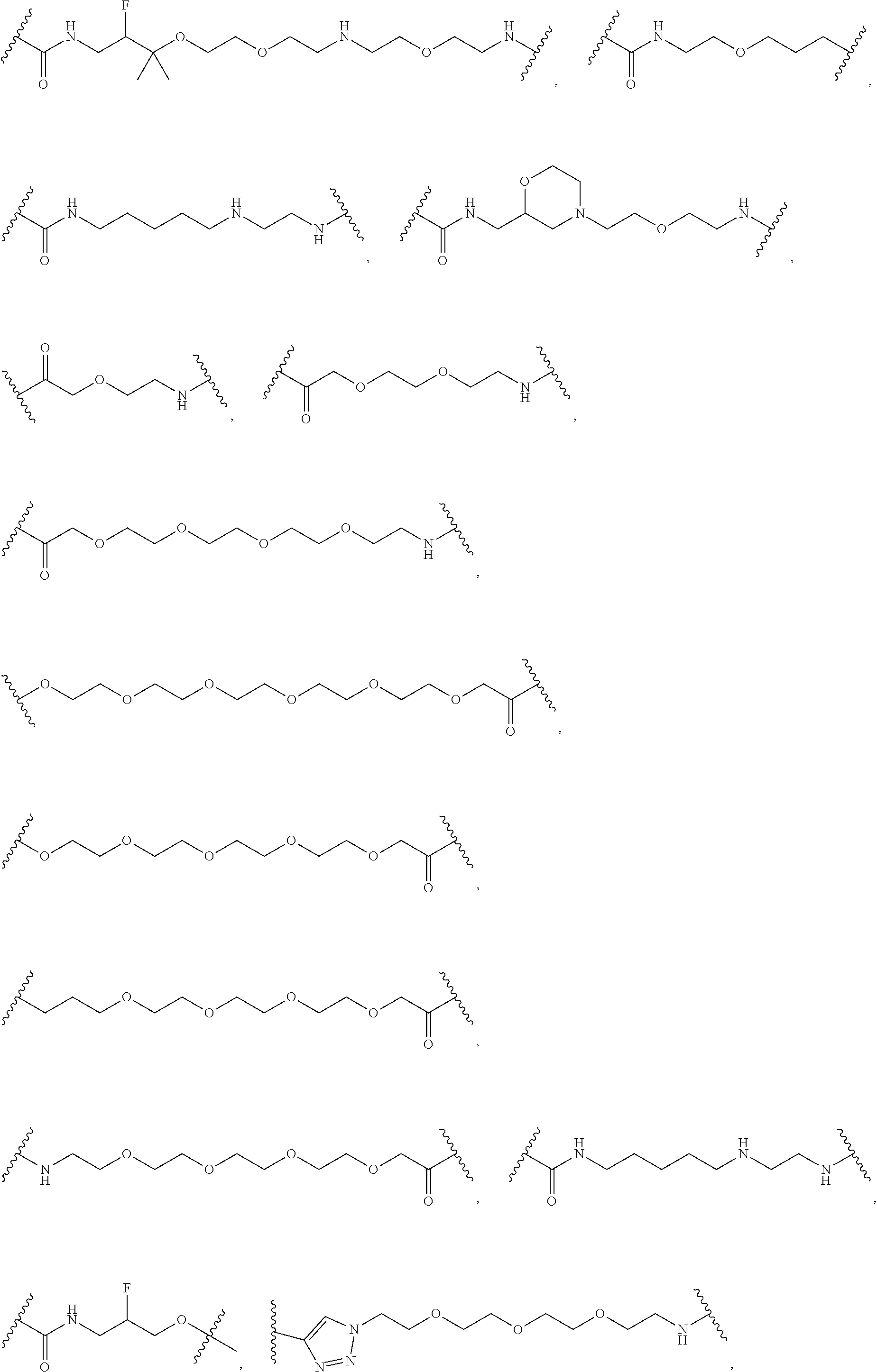

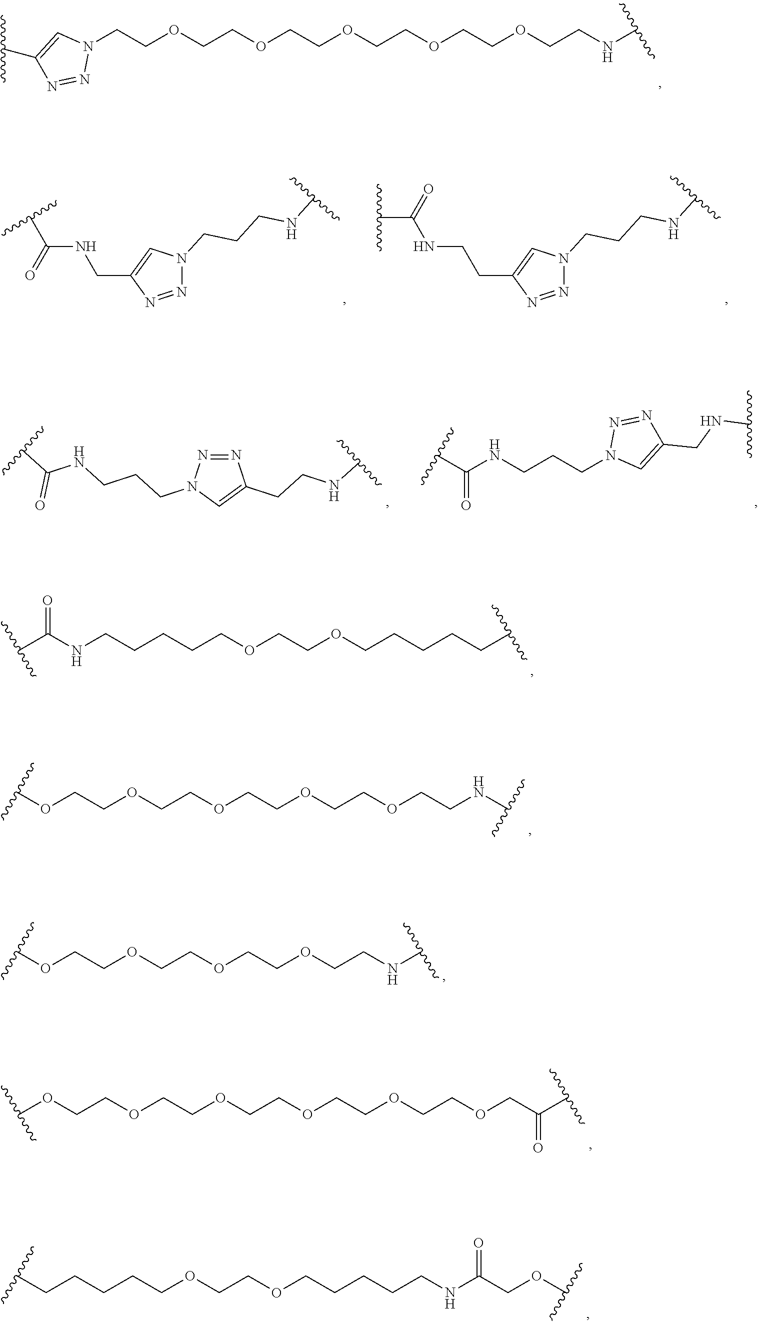

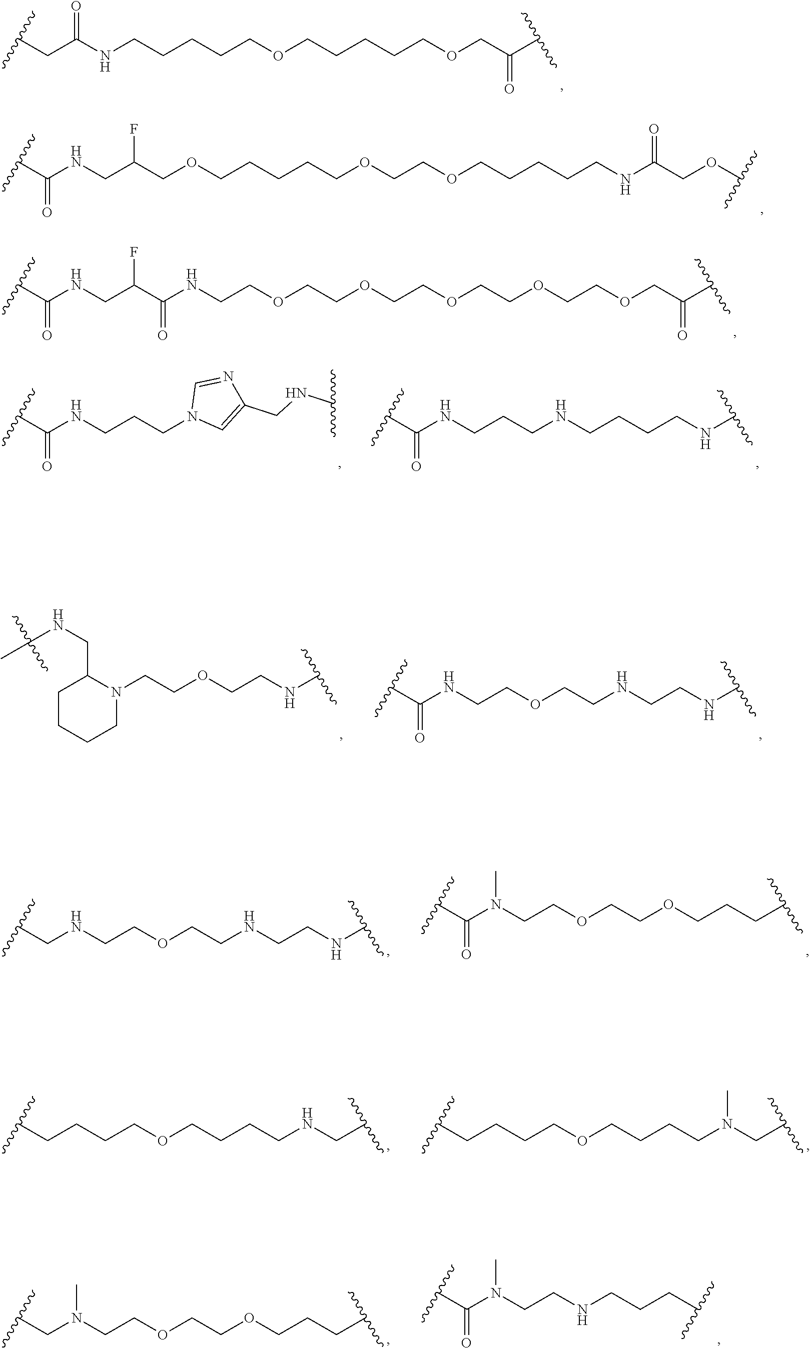

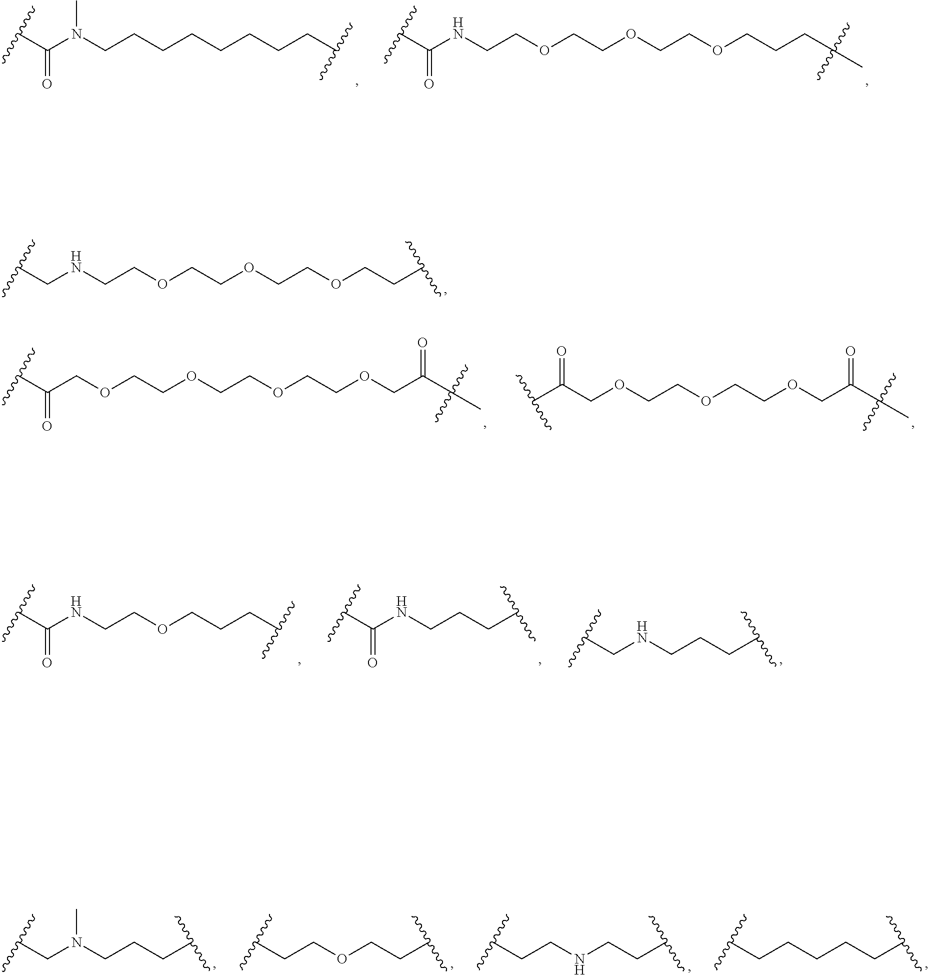

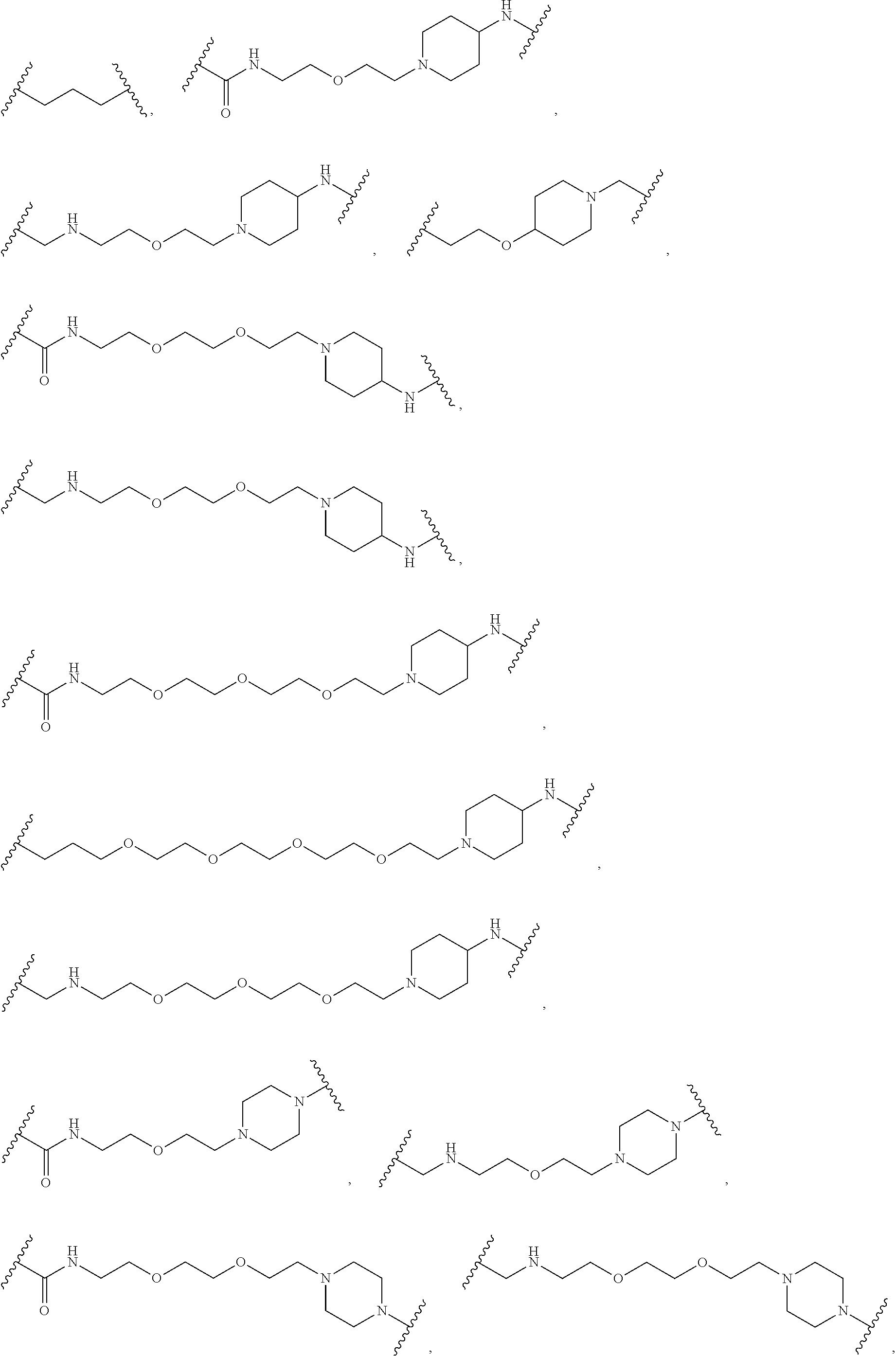

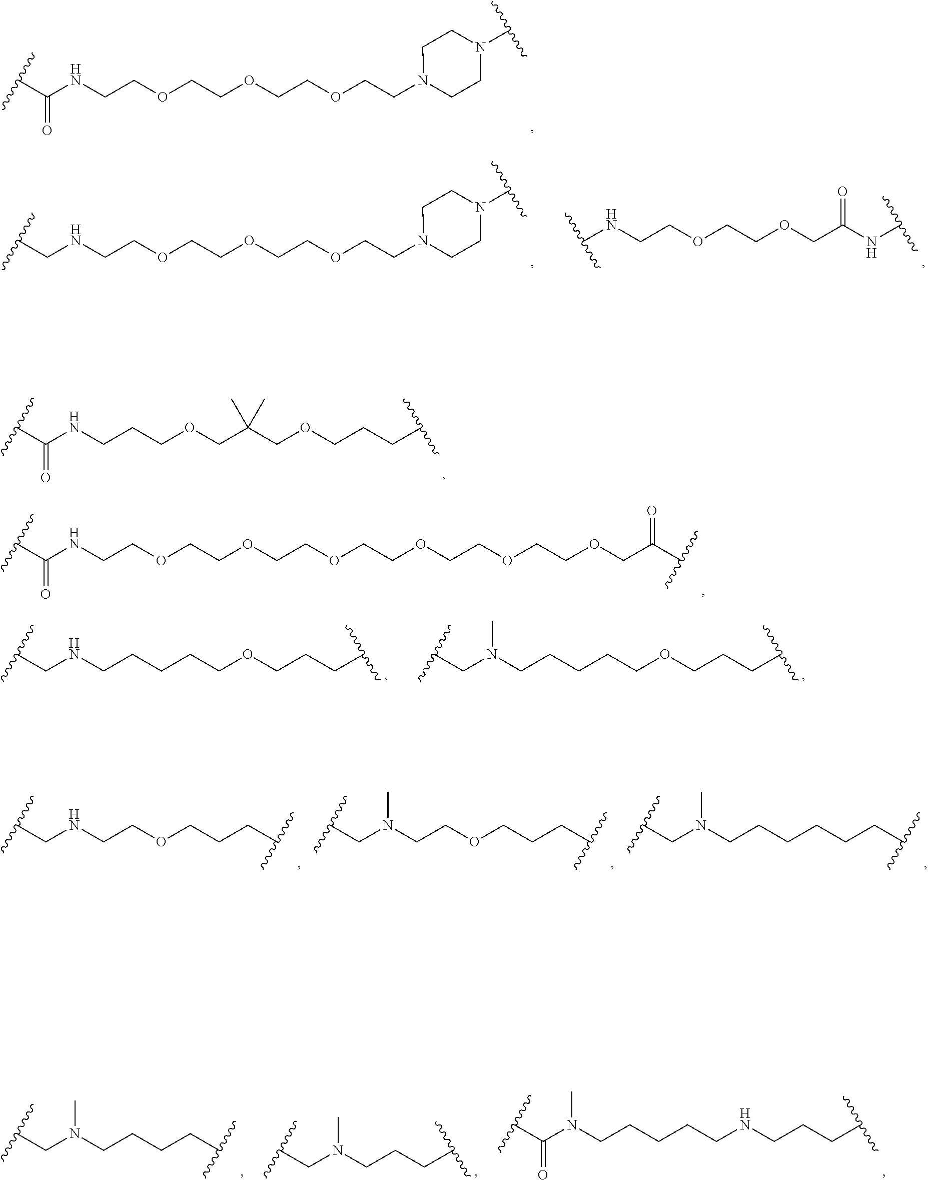

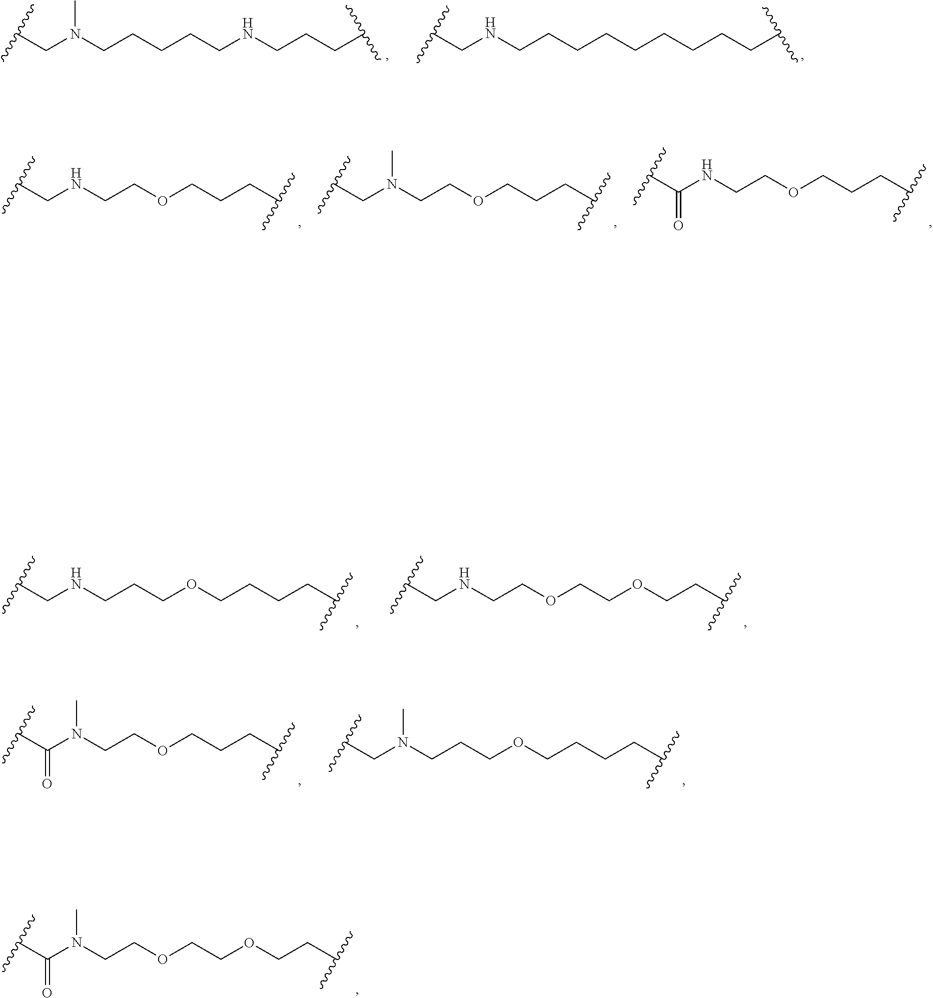

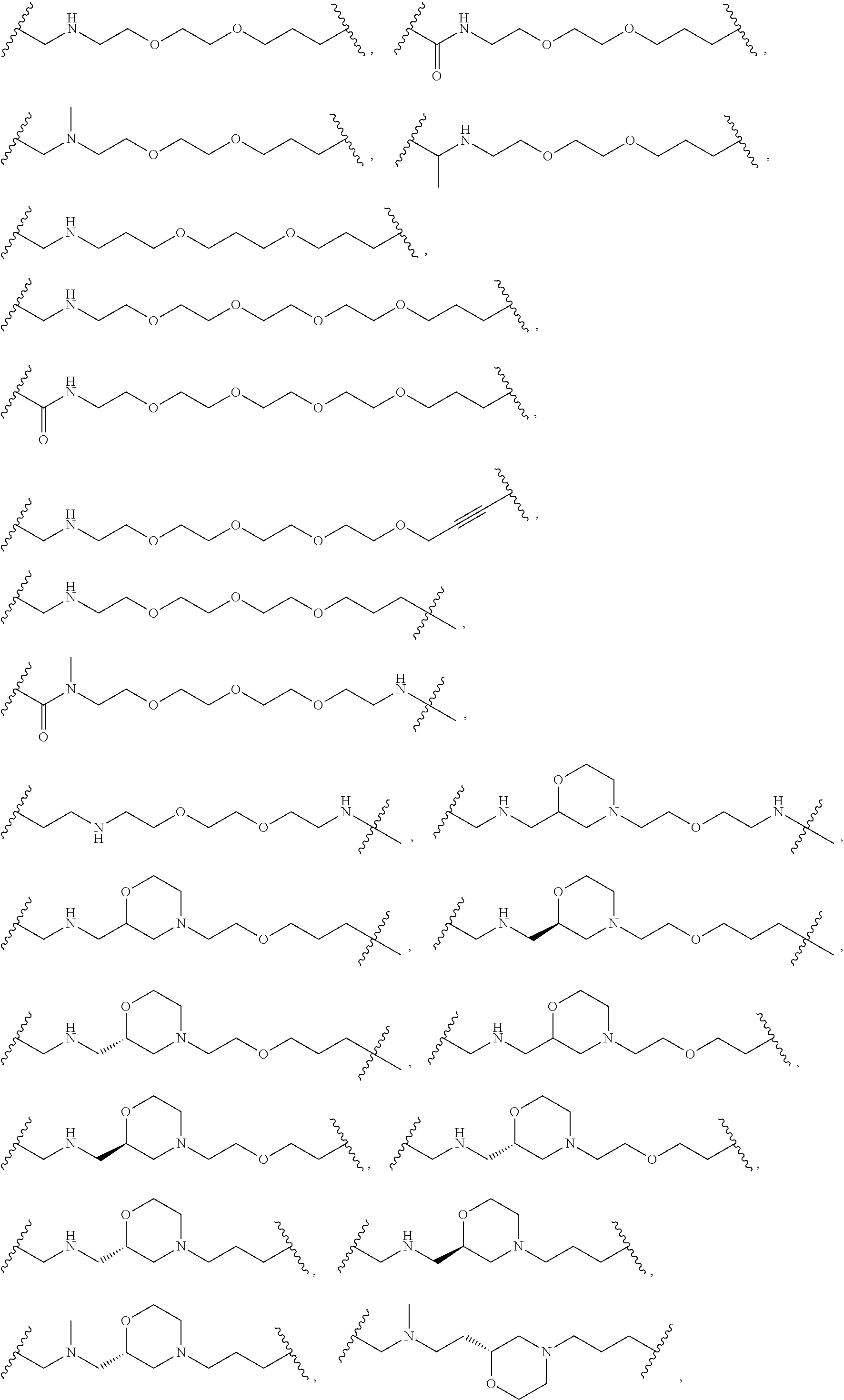

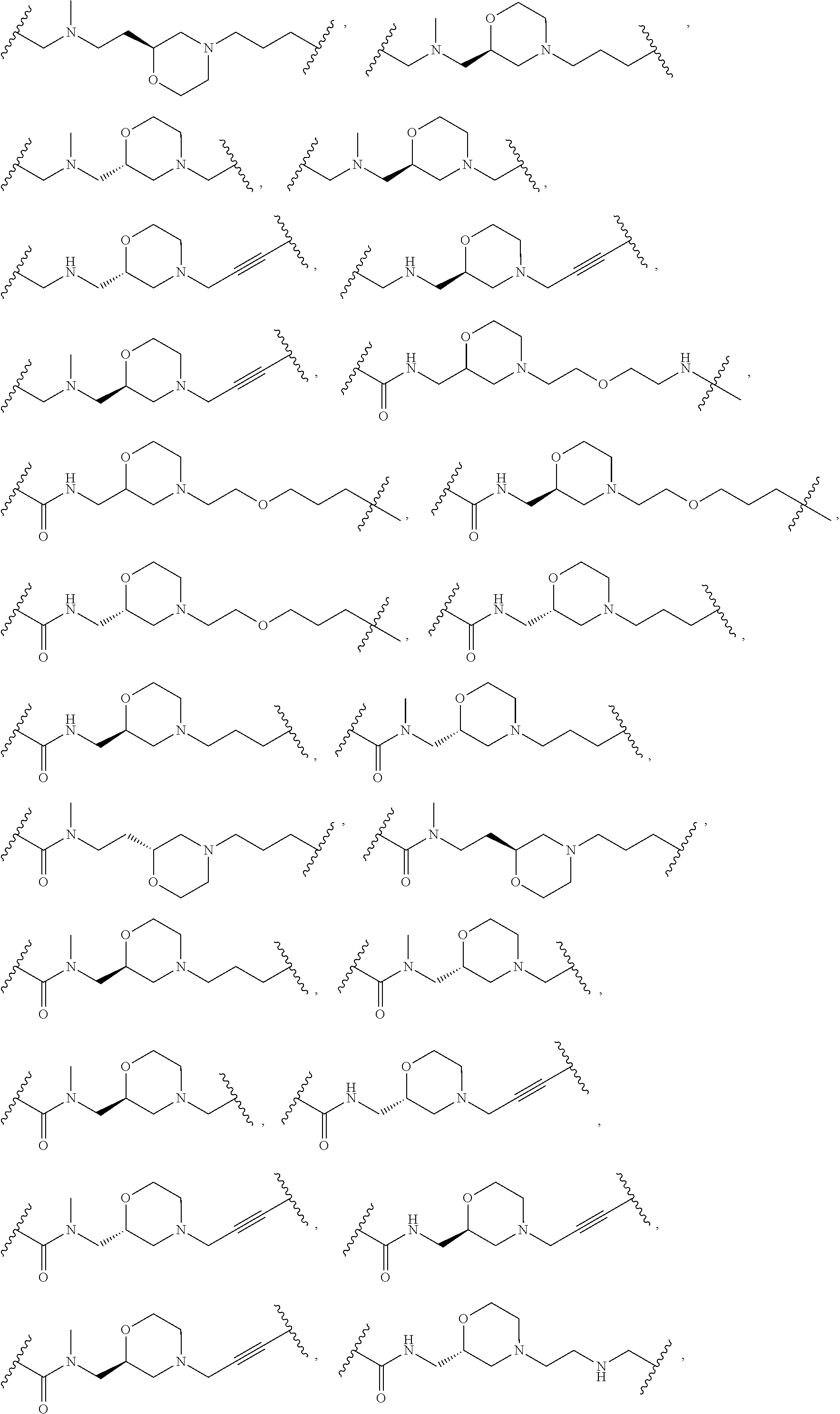

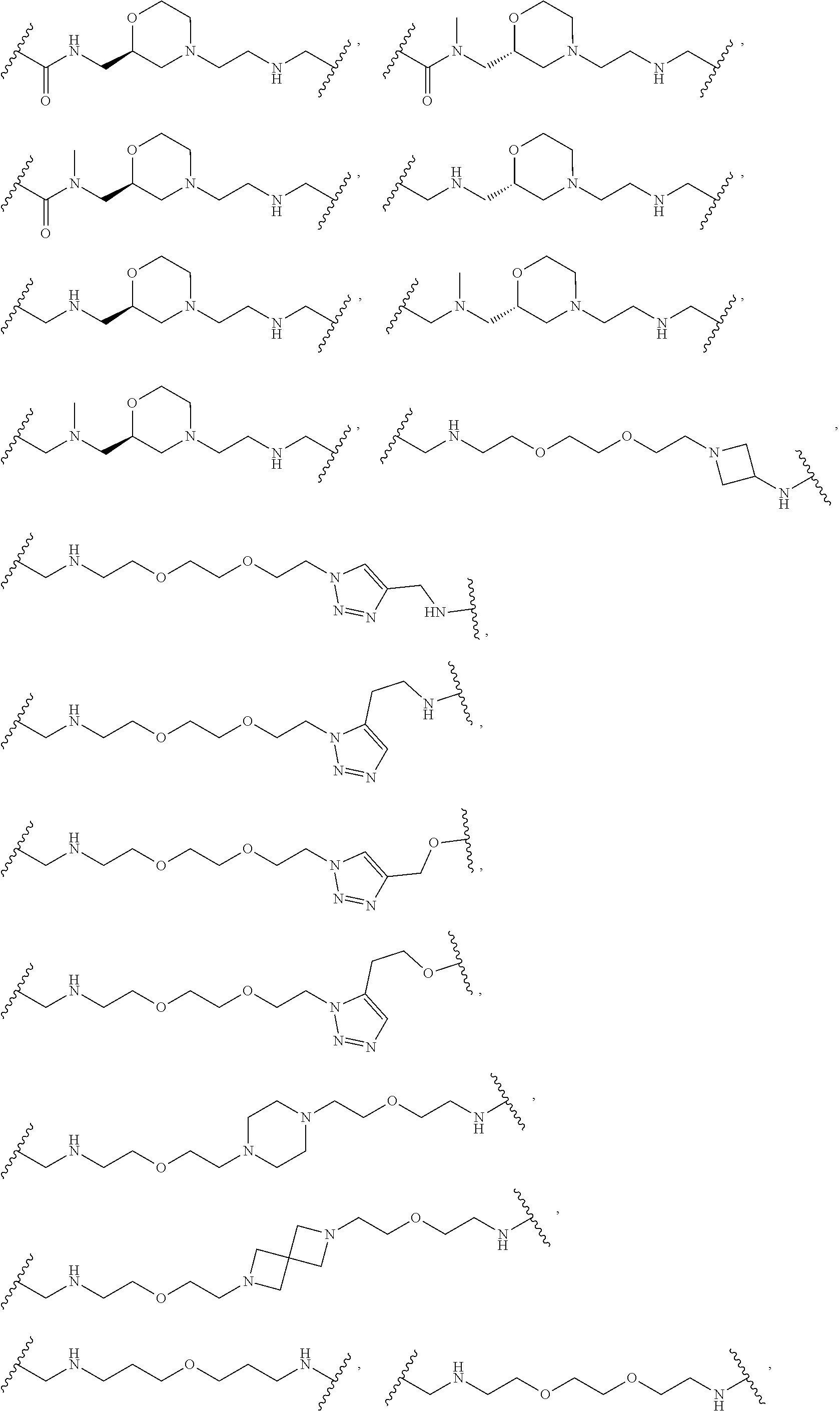

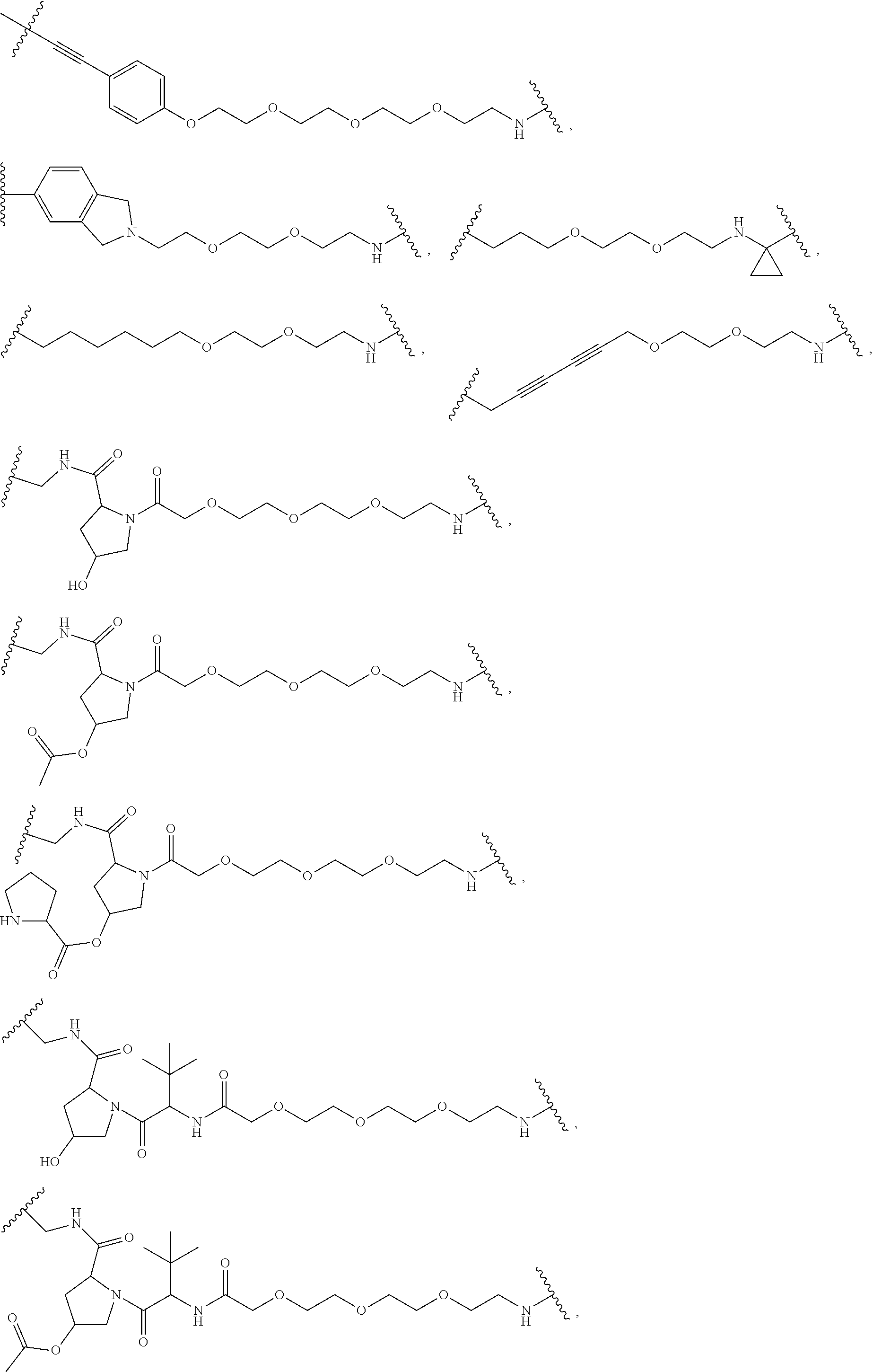

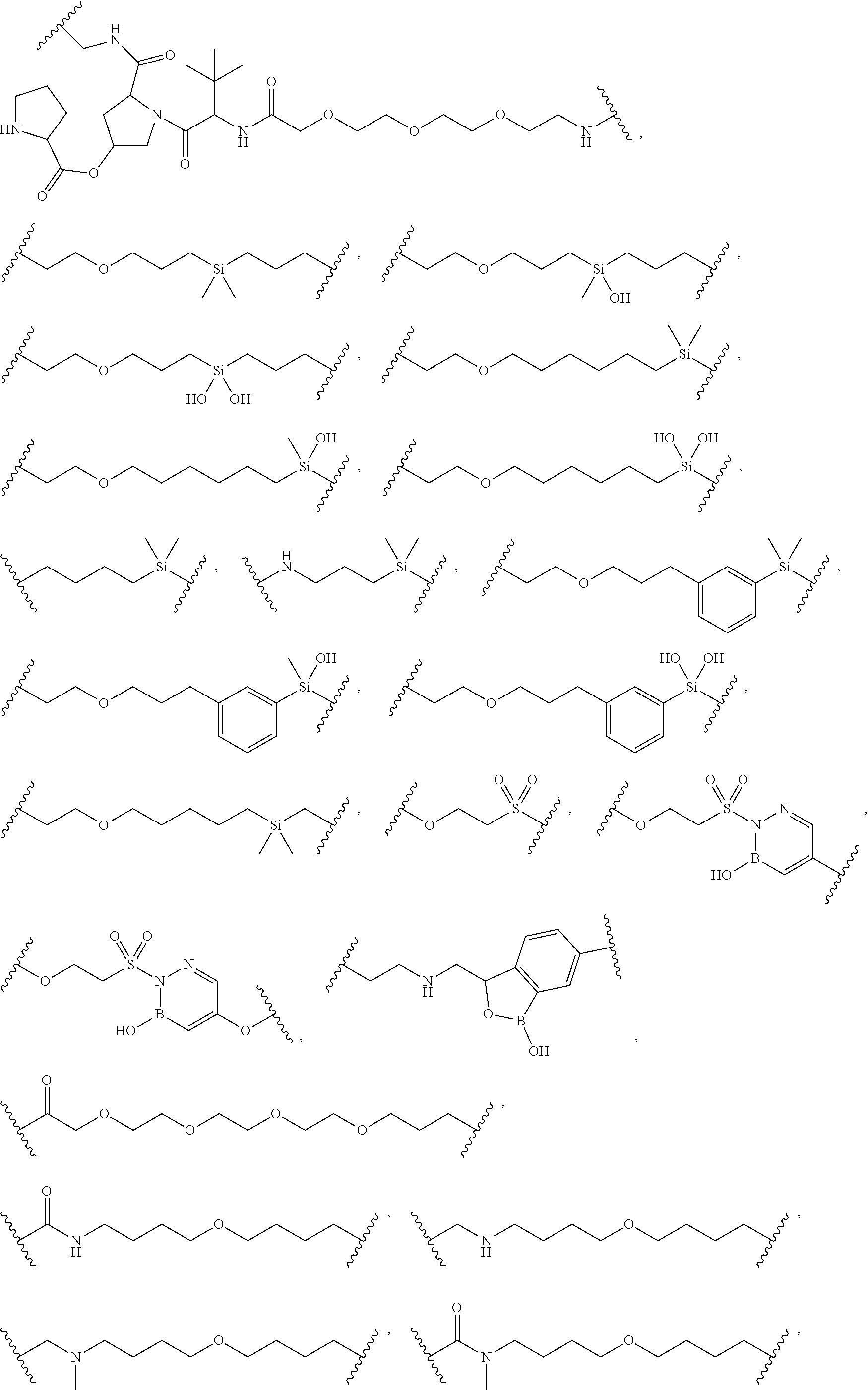

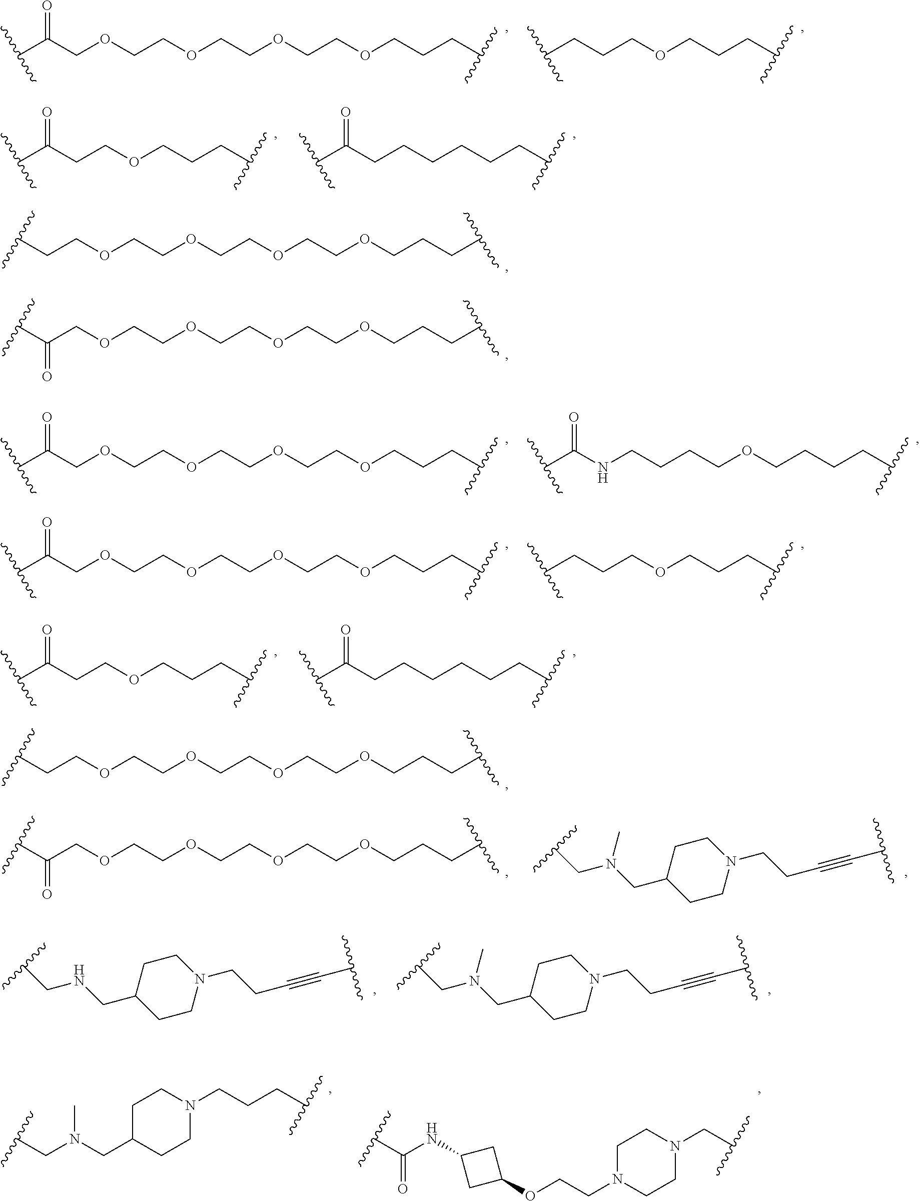

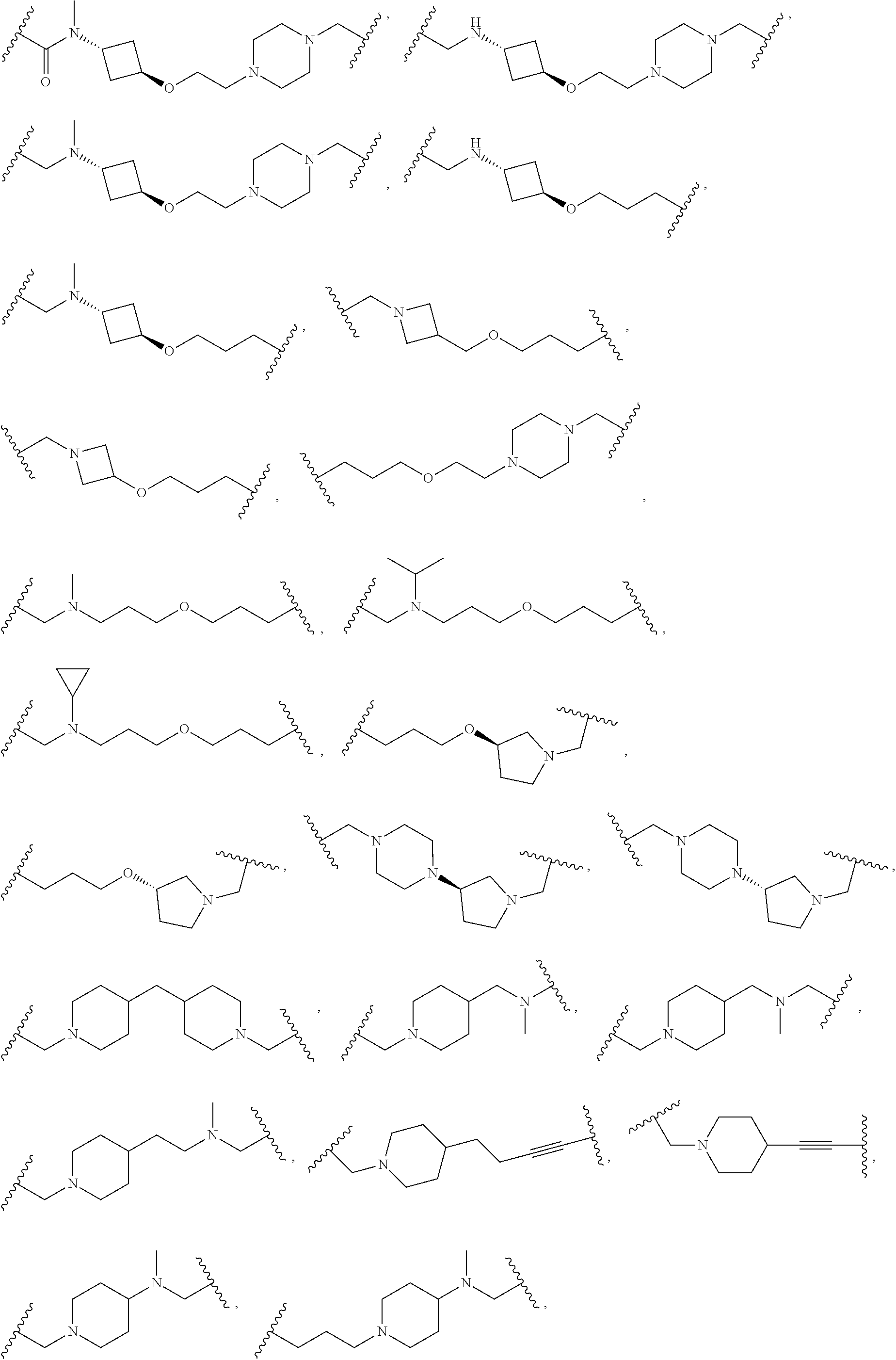

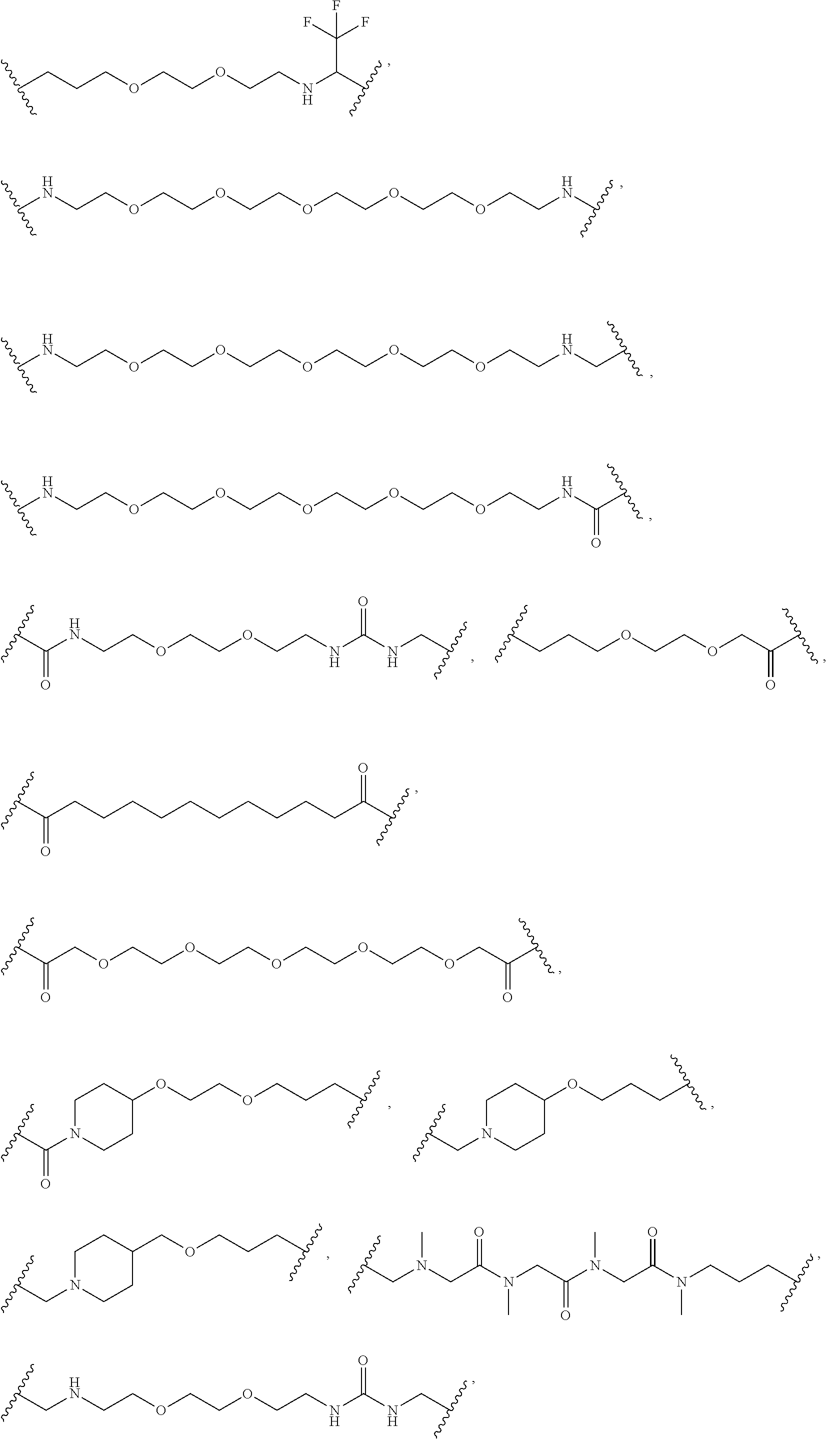

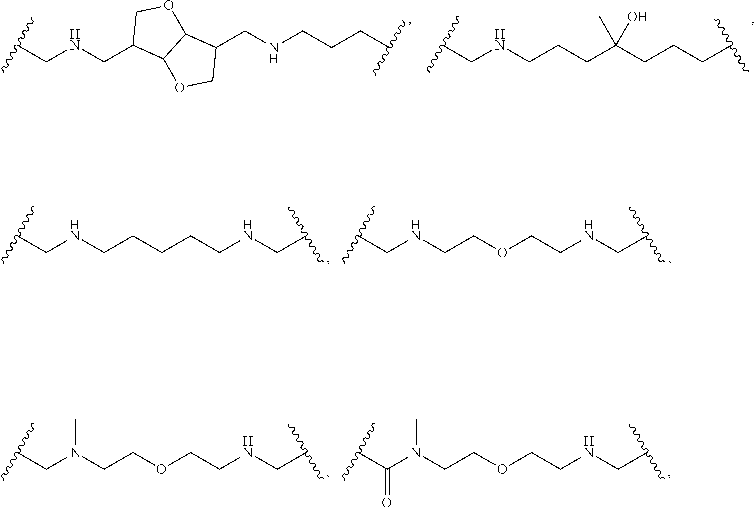 [SMILES: *CCCNCC1COC2C(CNC*)COC12 ; *CCCC(C)(O)CCCNC* ; *CNCCCCCNC* ; *CNCCOCCNC* ; *CNCCOCCN(C)C* ; *CNCCOCCN(C)C(*)=O]

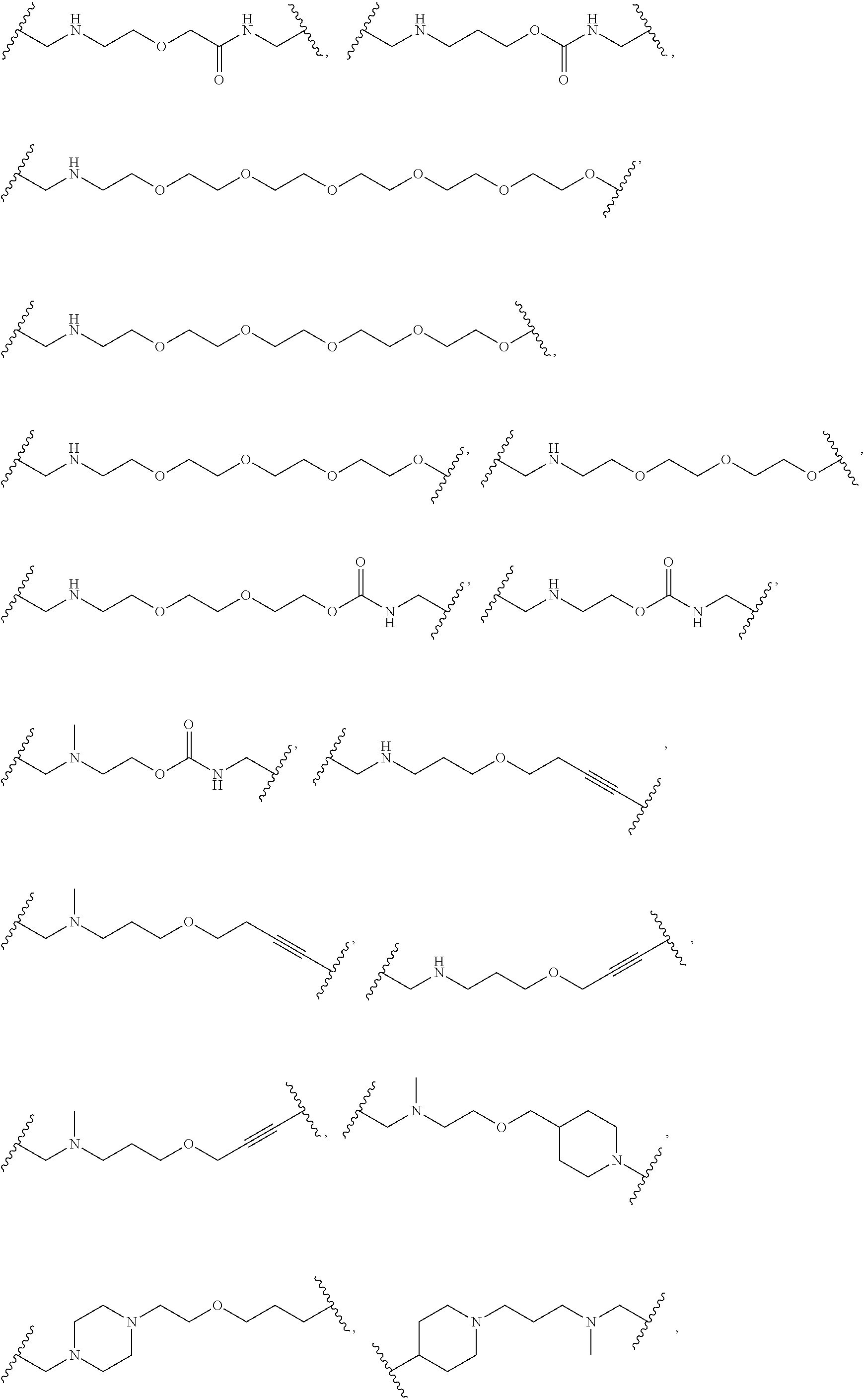

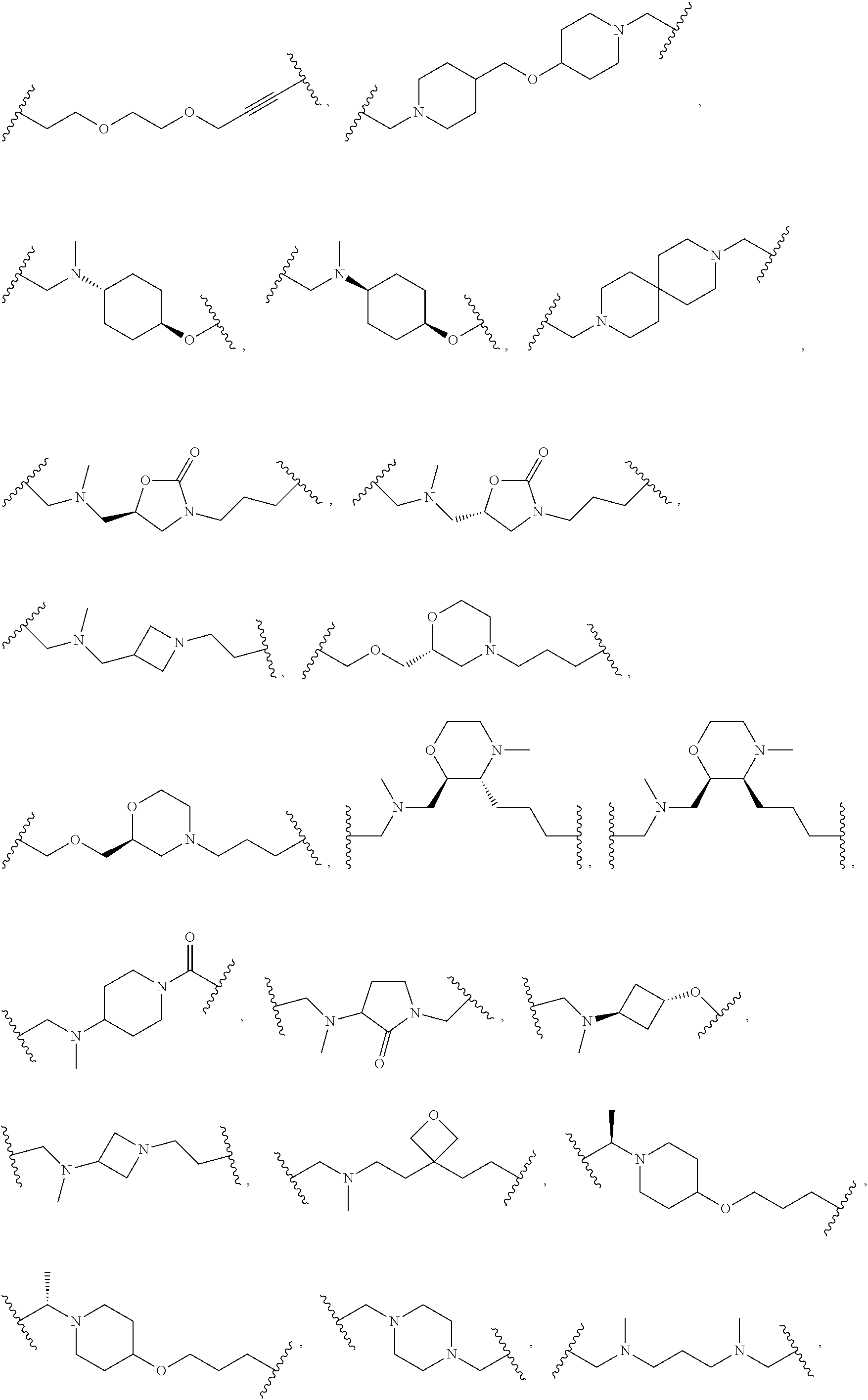

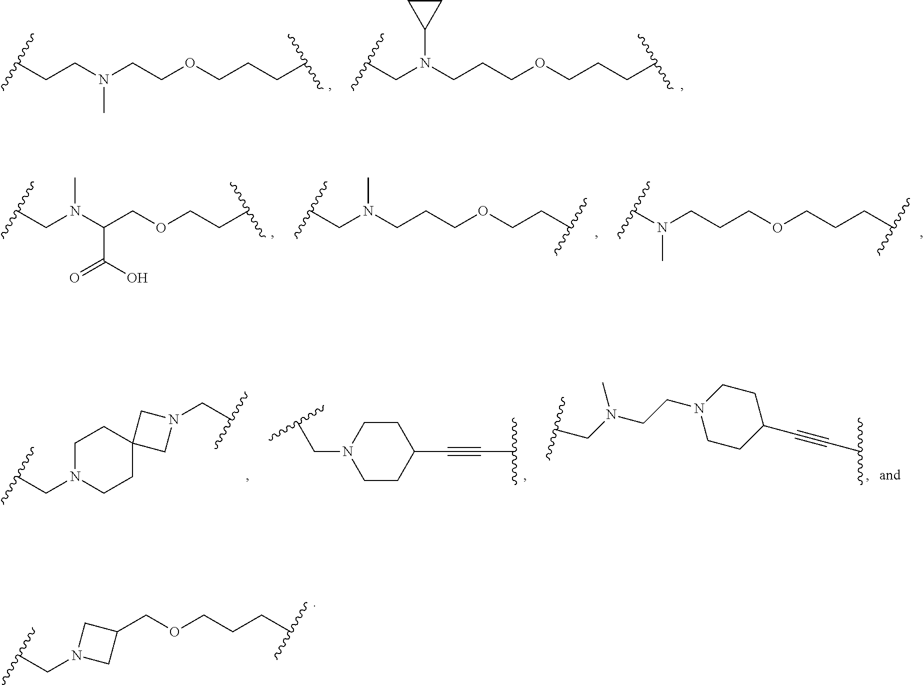

17. The bifunctional degrader of claim 1, wherein the protein of interest is FKBP12.

18. The bifunctional degrader of claim 17, wherein ligand A is SLF, having the structure (A-1):

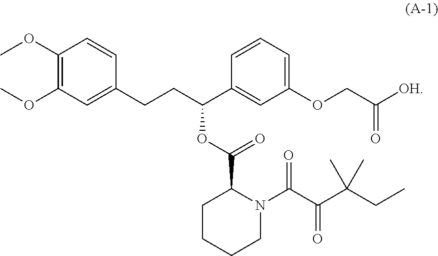 [SMILES: CCC(C)(C)C(=O)C(=O)N1CCCC[C@H]1C(=O)O[C@H](CCc1ccc(OC)c(OC)c1)c1cccc(OCC(=O)O)c1]

19. The bifunctional degrader of claim 17, wherein the linker B is

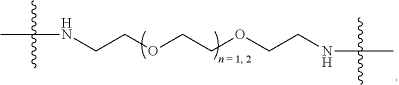

20. The bifunctional degrader of claim 17, having the structure: 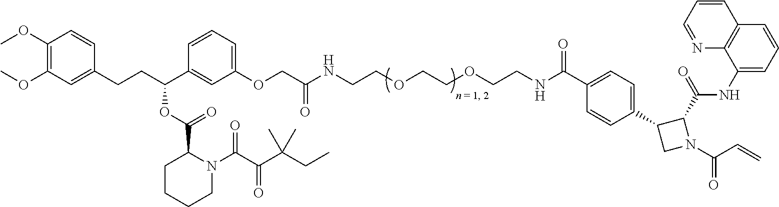
or a pharmaceutically acceptable salt thereof.

21. The bifunctional degrader of claim 1, wherein the protein of interest is the androgen receptor.

22. The bifunctional degrader of claim 21, wherein ligand A comprises at least one chemical moiety selected from the group consisting of: 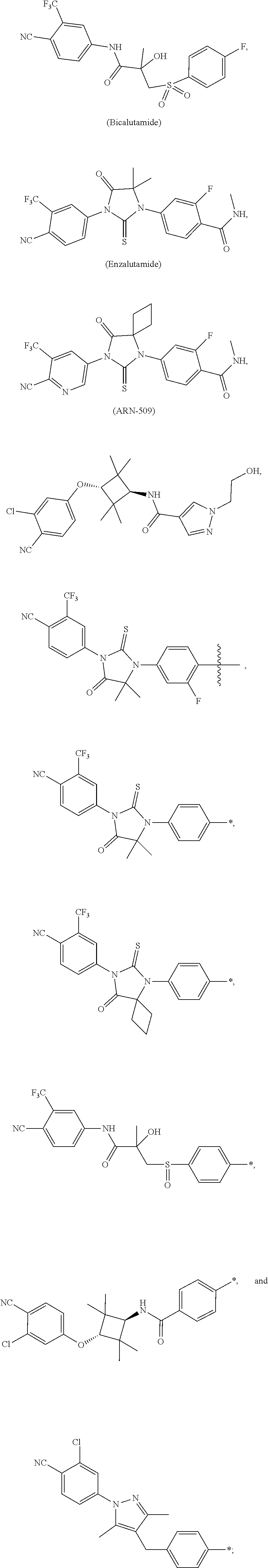
wherein in each instance, [symbol] or * can be a point of attachment for the linker B.

23. The bifunctional degrader of claim 1, wherein the protein of interest is the estrogen receptor.

24. The bifunctional degrader of claim 23, wherein ligand A comprises at least one chemical moiety selected from the group consisting of: 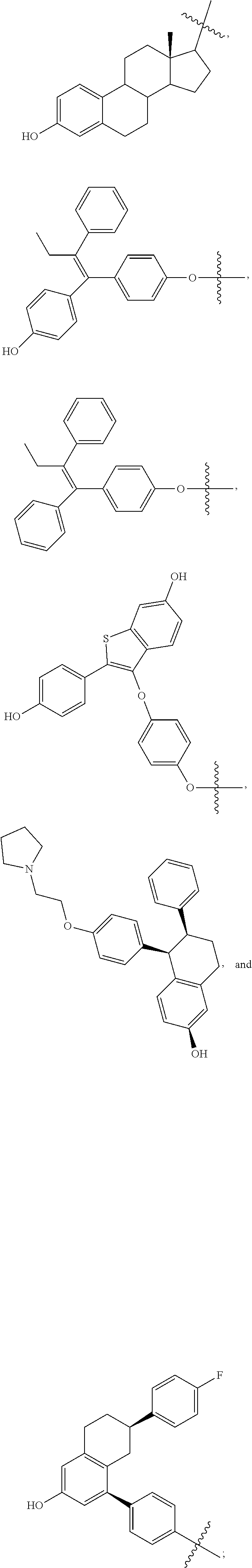
wherein [symbol] can be a point of attachment for the linker B.

25. The bifunctional degrader of claim 1, wherein the protein of interest is IRAK4.

26. The bifunctional degrader of claim 25, wherein ligand A comprises at least one chemical moiety selected from the group consisting of: 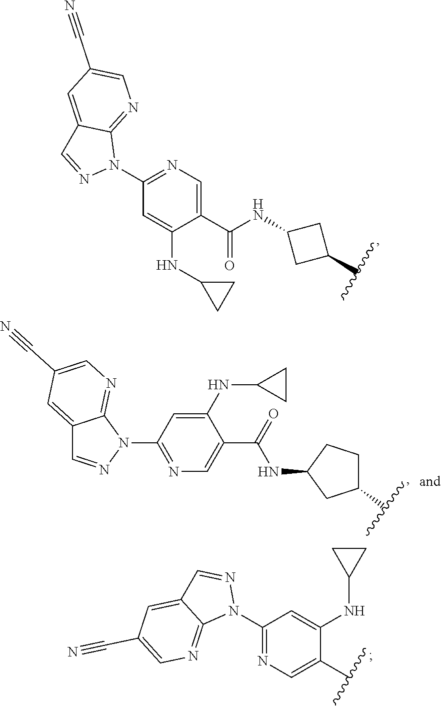
wherein [symbol] can be a point of attachment for the linker B.

27. The bifunctional degrader of claim 1, wherein the protein of interest is a JAK protein.

28. The bifunctional degrader of claim 27, wherein the JAK protein is JAK1, JAK2, or JAK3.

29. The bifunctional degrader of claim 27, wherein ligand A comprises at least one chemical moiety selected from the group consisting of: 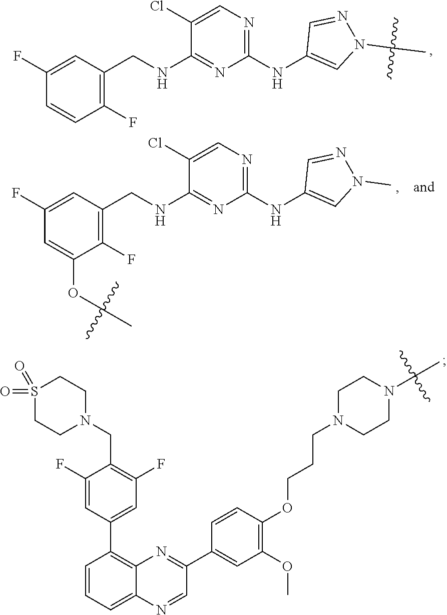
wherein [symbol] can be a point of attachment for the linker B.

30. The bifunctional degrader of claim 1, wherein the protein of interest is BCL-XL or BCL-2.

31. The bifunctional degrader of claim 30, wherein ligand A comprises at least one chemical moiety selected from the group consisting of: 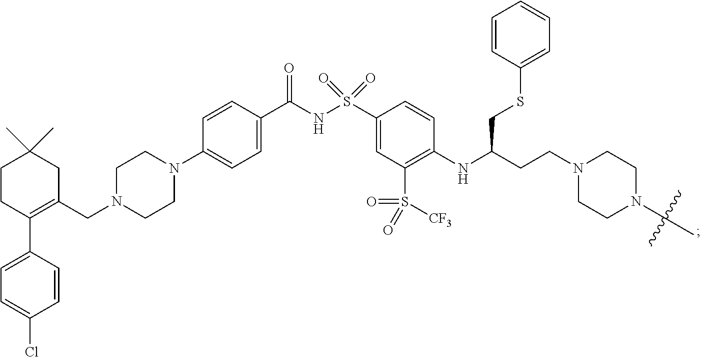 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]
wherein [symbol] can be a point of attachment for the linker B.

32. The bifunctional degrader of claim 1, wherein the protein of interest is Stat3.

33. The bifunctional degrader of claim 32, wherein ligand A comprises at least one chemical moiety of Formula (A-2): 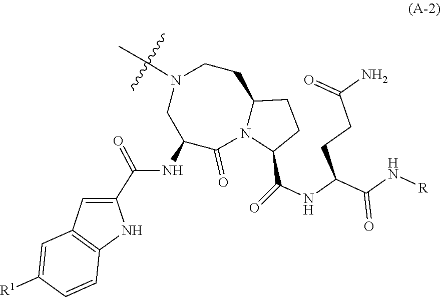
wherein:
R is: 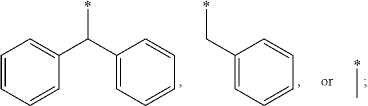
and
R¹ is: 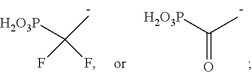
wherein * is the point of attachment for R and R¹, and [symbol] can be a point of attachment for the linker B.

34. The bifunctional degrader of claim 1, wherein the protein of interest is BRD4.

35. The bifunctional degrader of claim 34, wherein ligand A comprises at least one chemical moiety selected from the group consisting of: 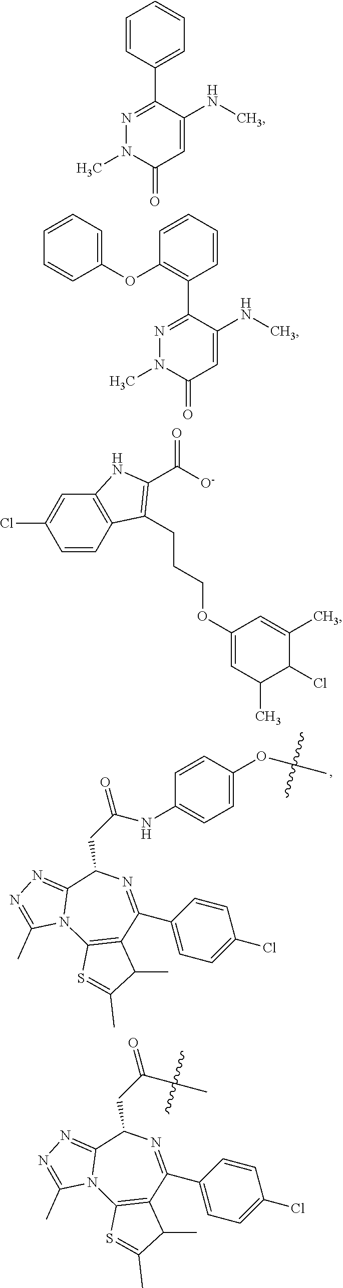 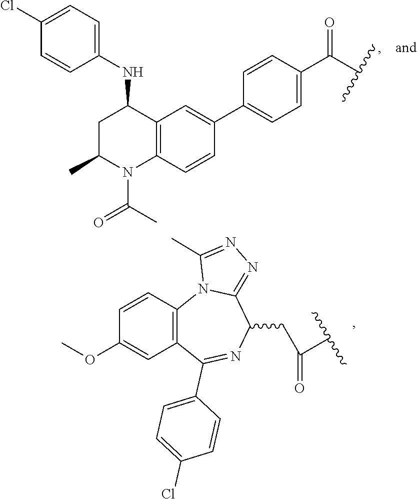
wherein [symbol] can be a point of attachment for the linker B.

36. The bifunctional degrader of claim 34, wherein ligand A is 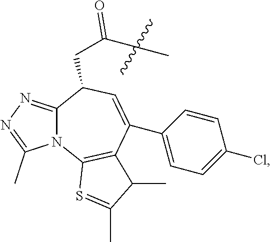
and the linker B is

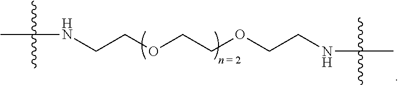

37. The bifunctional degrader of claim 36, having the structure:

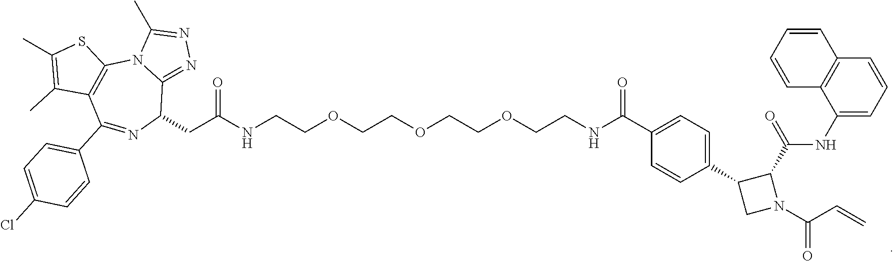 [SMILES: C=CC(=O)N1C[C@H](c2ccc(C(=O)NCCOCCOCCOCCNC(=O)C[C@@H]3N=C(c4ccc(Cl)cc4)c4c(sc(C)c4C)-n4c(C)nnc43)cc2)[C@@H]1C(=O)Nc1cccc2ccccc12]

38. A pharmaceutical composition comprising the bifunctional degrader of claim 1 or a pharmaceutically acceptable salt thereof, and a pharmaceutically acceptable carrier.

39. The pharmaceutical composition of claim 38, further comprising an additional therapeutic agent.

40. A method of degrading a protein selected from the group consisting of FKBP12, BRD4, androgen receptor, estrogen receptor, IRAK4, a JAK protein, BCL-XL, BCL-2, and Stat3 in a patient or biological sample, comprising administering to said patient, or contacting said biological sample, with the bifunctional degrader of claim 1.

41. A method of treating a disorder, disease or condition mediated by a protein selected from the group consisting of FKBP12, BRD4, androgen receptor, estrogen receptor, IRAK4, a JAK protein, BCL-XL, BCL-2, and Stat3, in a patient, comprising administering to the patient a therapeutically effective amount of the degrader of claim 1 or a pharmaceutically acceptable salt thereof.

42. The method of claim 41, wherein the disorder, disease or condition is a cancer, a neurodegenerative disease, a viral disease, an autoimmune disease, an inflammatory disorder, a hereditary disorder, a hormone-related disease, a hematopoietic disorder, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, and a CNS disorder.

43. The method of claim 41, further comprising administering an additional therapeutic agent.

44. A DCAF1 protein-probe adduct, wherein the probe binds to cysteine residue C1113 of DCAF1, wherein the amino acid numbering is based on DCAF1 Isoform 1 (Accession No. Q9Y4B6-1), cysteine residue C1112 of DCAF1, wherein the amino acid numbering is based on DCAF1 Isoform 2 (Accession No. Q9Y4B6-2), or cysteine residue C664 of DCAF1, wherein the amino acid numbering is based on DCAF1 Isoform 3 (Accession No. Q9Y4B6-3) and wherein the probe comprises an azetidinyl acrylamide moiety.

45. The DCAF1 protein-probe adduct of claim 44, wherein the probe is a compound of Formula (I): 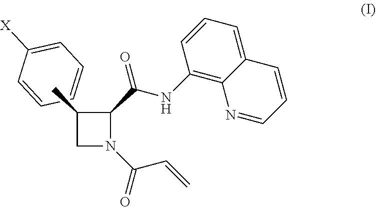
wherein:
X is selected from the group consisting of halo,

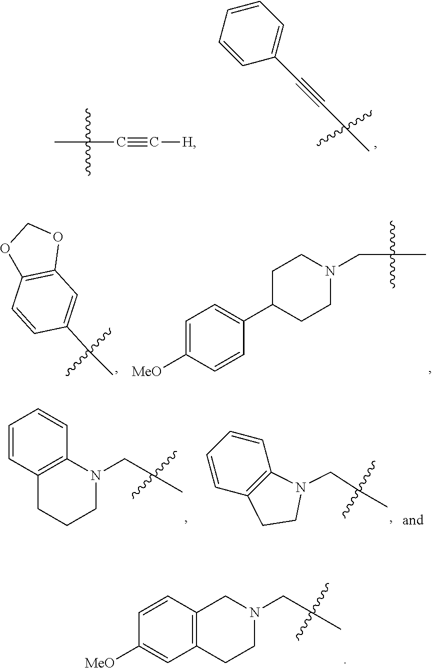

46. The DCAF1 protein-probe adduct of claim 45, having the structure of Formula (II): 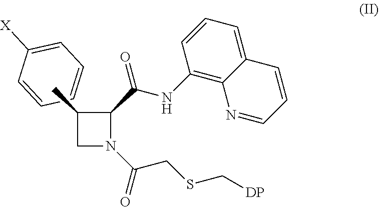
wherein:
S represents the sulfur atom of a cysteine residue C1113, cysteine residue C1112, or cysteine residue C664; and
DP represents the DCAF1 polypeptide.

47. The DCAF1 protein-probe adduct of claim 44, wherein the probe is selected from the group consisting of:

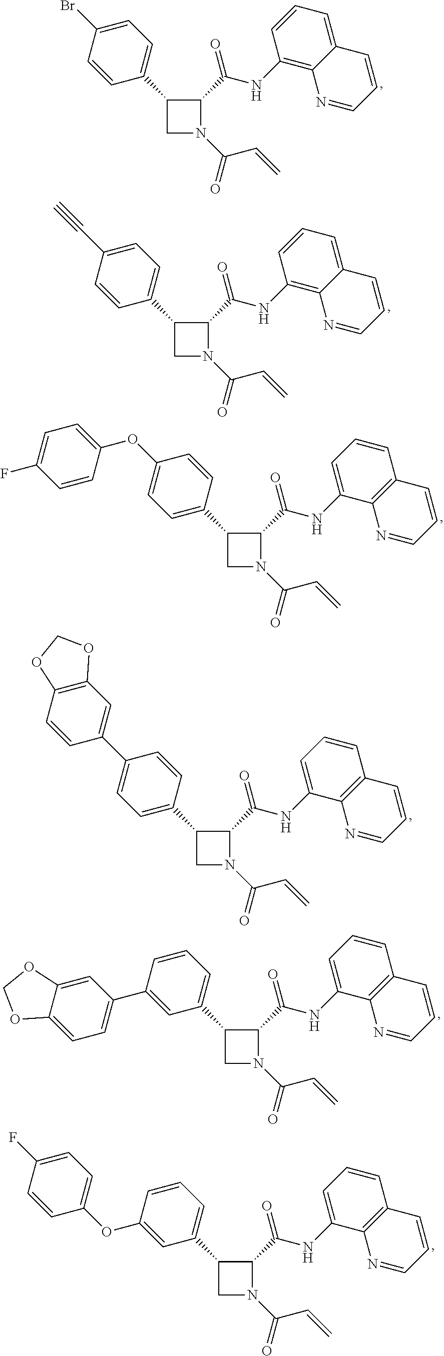 [SMILES: C=CC(=O)N1C[C@H](c2ccc(Br)cc2)[C@@H]1C(=O)Nc1cccc2cccnc12 ; C#Cc1ccc([C@H]2CN(C(=O)C=C)[C@H]2C(=O)Nc2cccc3cccnc23)cc1 ; C=CC(=O)N1C[C@H](c2ccc(Oc3ccc(F)cc3)cc2)[C@@H]1C(=O)Nc1cccc2cccnc12 ; C=CC(=O)N1C[C@H](c2ccc(-c3ccc4c(c3)OCO4)cc2)[C@@H]1C(=O)Nc1cccc2cccnc12 ; C=CC(=O)N1C[C@H](c2cccc(-c3ccc4c(c3)OCO4)c2)[C@@H]1C(=O)Nc1cccc2cccnc12 ; C=CC(=O)N1C[C@H](c2cccc(Oc3ccc(F)cc3)c2)[C@@H]1C(=O)Nc1cccc2cccnc12]

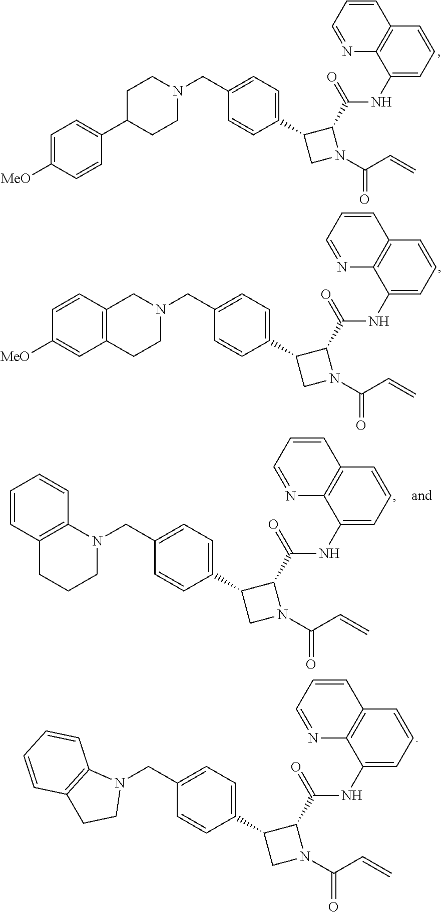

48. A compound of Formula (I) 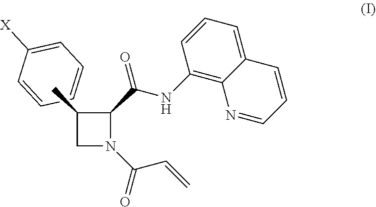
or a pharmaceutically acceptable salt thereof, wherein:
X is selected from the group consisting of halo,

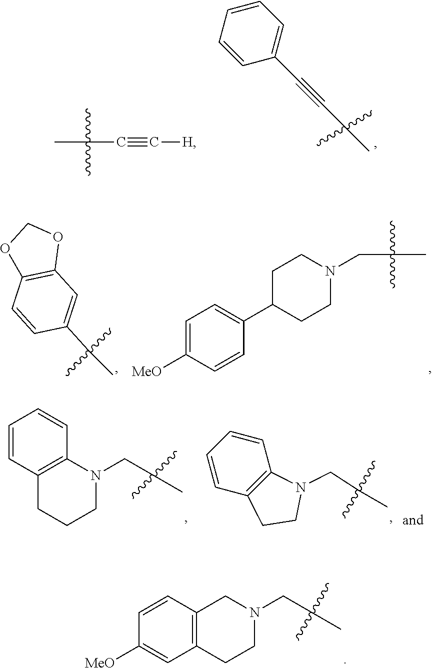

49. The compound of claim 48, selected from the group consisting of: 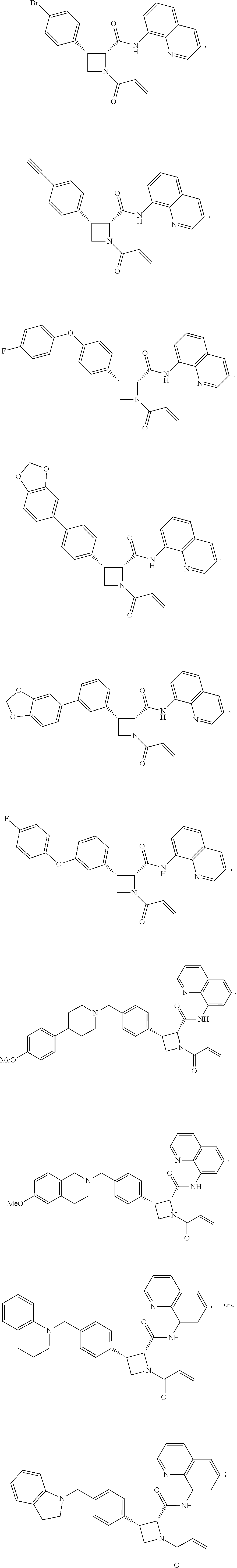
or a pharmaceutically acceptable salt thereof.

50. A method of agonizing or antagonizing DCAF1 protein, wherein the amino acid numbering is based on DCAF1 Isoform 1 (Accession No. Q9Y4B6-1), in a patient in need of such agonization or antagonization, or in a biological sample, comprising administering to the patient, or contacting the biological sample with the compound of claim 48, or a pharmaceutically acceptable salt thereof.
